(19) United States
(12) Patent Application Publication
(10) Pub. No.: US 2026/0258091 A1
(43) Pub. Date: Sep. 3, 2026

(54) NOVEL BACTERIAL TRANSLOCATION DOMAINS AND RECOMBINANT POLYPEPTIDES COMPRISING THEM FOR USE IN CELLULAR DELIVERY

(71) Applicant: The Hospital for Sick Children, Toronto (CA)

(72) Inventors: Roman MELNYK, Oakville (CA); Greg BEILHARTZ, Burlington (CA); Shivneet GILL, Brampton (CA); Seiji SUGIMAN-MARANGOS, Toronto (CA)

(21) Appl. No.: 18/706,517

(22) PCT Filed: Aug. 10, 2022
(86) PCT No.: PCT/CA2022/051225

§ 371 Date: May 1, 2024

(30) Foreign Application Priority Data
Nov. 4, 2021 (GR) 20210100770

(51) Int. Cl. C07K 14/195; A61K 47/64; A61P 35/00; C07K 19/00

(52) U.S. Cl. CPC C07K 14/195; A61K 47/64; A61P 35/00; C07K 19/00

(57) ABSTRACT

Generally, the present disclosure provides novel bacterial translocation domains for use in cellular delivery. An example is the translocation domain from the Austwickia chelonae protein of SEQ ID NO:2, the translocation domain having the amino acid sequence of SEQ ID NO: 3. Translocase domains from other bacterial strains and species are also described (e.g. SEQ ID NOs: 4 to 17 and 36 to 48). Recombinant polypeptides comprising these translocation domains are described. The recombinant polypeptides are intended for use in delivery of cargo molecules, including therapeutic polypeptides that may be used to treat disease.

### CROSS REFERENCE TO RELATED APPLICATIONS

This application claims the benefit of priority from Greek patent application Ser. No. 20210100770 entitled “NOVEL BACTERIAL TRANSLOCATION DOMAINS AND RECOMBINANT POLYPEPTIDES COMPRISING THEM FOR USE IN CELLULAR DELIVERY”, which was filed on Nov. 4, 2021.

### SEQUENCE LISTING

The application contains a sequence listing which has been submitted electronically in XML format and is hereby incorporated by reference in its entirety. Said XML copy, created on Apr. 9, 2026, is named “PAT111957W-2_SequenceListing.xml”, and is 57,500 bytes in size.

### FIELD

The present disclosure relates generally to a delivery platform. More particularly, the present disclosure relates to a bacterial toxin-based platform for delivery of cargo molecules to cells.

### BACKGROUND

Therapeutic molecules are often difficult to deliver to cells. They often do not readily penetrate biological membranes. Immunotoxins are a class of biotherapeutics comprised of bacterial toxins, such as diphtheria toxin (DT), that have been repurposed, e.g., into cancer-targeted therapies-both by re-targeting their receptor binding domains (RBD) to target cancer receptors, and by delivering enzyme cargo that target intracellular oncoproteins. However, global vaccination programs against diphtheria has resulted in population-level immunity against DT, and DT-based therapeutics.

There remains a need for delivery platforms that offer the possibility to deliver therapeutics, including protein-based therapeutics, to cells.

### SUMMARY

It is an object of the present disclosure to obviate or mitigate at least one disadvantage of previous approaches.

In a first aspect, the present disclosure provides a recombinant polypeptide of general formula (I):

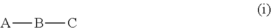

wherein:
A is a cargo molecule;
B is a translocation polypeptide comprising:
a) a translocation domain from:
the Austwickia chelonae protein of SEQ ID NO: 2,
the Streptosporangium nondiastaticum protein of GenBank Accession PSJ28985.1,
the Streptomyces sp. TLI 053 protein of GenBank Accession SDT83331.1,
the Streptomyces sp. SLBN-118 protein of GenBank Accession WP_160159328.1,
the Streptomyces sp. AA8 protein of GenBank Accession WP_168096531.1,
the Streptomyces roseoverticillatus protein of GenBank Accession WP_078659863.1,
the Streptomyces piniterrae protein of GenBank Accession JZ58907.1,
the Streptomyces MBT76 protein of GenBank Accession WP_079110321.1,
the Streptomyces klenkii protein of GenBank Accession WP_120757473.1,
the Streptomyces albireticuli protein of GenBank Accession WP_095582082.1,
the Streptacidiphilus pinicola protein of GenBank Accession WP_133259917.1,
the Seinonella peptonophila protein of GenBank Accession WP_073156187.1,
the Longimycelium tulufanense protein of GenBank Accession WP_189053160.1,
the Austwickia sp. TVS 96-490-7B protein of GenBank Accession WP_219106995.1,
the Austwickia chelonae LK16-18 protein of GenBank Accession WP_162873017.1;
the Klebsiella aerogenes protein of GenBank Accession EIZ2913133.1,
the Streptomyces sp. MUM 178J protein of GenBank Accession MCH0551590.1,
the Crossiella cryophila protein of GenBank Accession MBB4677777.1,
the Allokutzneria sp. NRRL B-24872 protein of GenBank Accession WP_143261759.1,
the Allokutzneria albata protein of GenBank Accession WP_156051914.1,
the Streptomyces sp. AV19 protein of GenBank Accession WP_199893204.1,
the Streptomyces sp. NRBC_110611 protein of GenBank Accession WP_147264604.1,
the Streptomyces syringium protein of GenBank Accession WP_209513619.1,
the Pseudonocardiaceae bacterium YIM PH 21723 protein of GenBank Accession RJQ69589.1,
the Actinokineospora bangkokensis protein of GenBank Accession WP_143218892.1,
the Streptomyces eurocidicus protein of GenBank Accession MBF6055834.1,
the Streptomyces pathocidini protein of GenBank Accession WP_169790908.1, or
the Streptomyces caatingaensis protein of GenBank Accession WP_157868472.1, or
b) a translocation domain that is at least 80% identical to the translocation domain defined in a); and
C is a targeting moiety.

In one aspect, there is provided a nucleic acid encoding the recombinant polypeptide as defined here.

In one aspect, there is provided a vector comprising the nucleic acid as defined herein.

In further aspect, the present disclosure provides a composition comprising the recombinant polypeptide as defined herein, together with an acceptable excipient, diluent, or carrier.

In one aspect embodiment, there is provided a pharmaceutical composition the recombinant polypeptide as defined herein, together with a pharmaceutically acceptable excipient, diluent, or carrier.

In one aspect, there is provided a method of delivery a cargo molecule to a cell comprising contacting the cell with the recombinant polypeptide as defined herein.

In one aspect, there is provided a use of the recombinant polypeptide as defined herein for delivery of the cargo molecule to a cell.

In one aspect, there is provided a use of the recombinant polypeptide as defined herein for preparation of a medicament for delivery of the cargo molecule to a cell.

In one aspect, there is provided the recombinant polypeptide as defined herein for use in delivery of the cargo molecule to a cell.

In one aspect, there is provided a method treating cancer in a subject comprising administering to the subject the recombinant polypeptide as defined herein.

In one aspect, there is provided a use of the recombinant polypeptide as defined herein for treatment of cancer in a subject.

In one aspect, there is provided a use of the recombinant polypeptide as defined herein for preparation of a medicament for treatment of cancer in a subject.

In one aspect, there is provided the recombinant polypeptide as defined herein for use in treatment of cancer in a subject.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIG. 1A depicts a schematic of the cell entry mechanism of diphtheria toxin.

FIG. 1B depicts the domain organization of DT and CT1 showing the catalytic domain (C) and the bridging furin recognition site (F), followed by the translocase (T) and receptor-binding domains (R).

FIG. 1C depicts the crystal structures of diphtheria toxin and CT1.

FIG. 2A shows results of experiments assessing a CT1 catalytic domain function and release. DT and a DT-chimera containing the C-domain of CT1 shows similar toxicity on HEK293T cells (b).

FIG. 2B shows that DT and a DT-chimera containing the C-domain of CT1 shows no effect on cell viability on DPH4^{-/-} cells.

FIG. 2C shows that when DT and CT1 were incubated with cell lysates, there was cleavage at the furin recognition site.

FIG. 3A shows that DT and a DT chimeric containing the furin site from CT1 are equipotent on Vero cells, demonstrating efficient cargo release by both toxins.

FIG. 3B shows reduced protein synthesis in Vero cells by CT1-T-mediated delivery of DT-C.

FIG. 3C shows CT1-T-mediated delivery of the non-native cargo RRSP. RRSP is toxic to RAS mutant cells such as CFPAC-1.

FIG. 3D shows that delivery was confirmed by the cleavage of intracellular RAS by RRSP.

FIG. 3E shows that CT1-T-mediated delivery of DT-C by measuring cell viability on HPAF II cells. Unexpectedly, CT1-T is a more efficient translocase than DT-T when complexed with targeting domains beyond DT-R.

FIG. 3F shows that CT1-T-mediated delivery of RRSP by measuring cell viability on HPAF II cells.

FIG. 4A shows results of experiments assessing CT1 human serum binding and neutralization. To quantify the level of pre-existing anti-DT or anti-CT1 antibodies in human serum, DT or CT1 was immobilized on Nunc MaxiSorp™ plates and incubated with human serum at various dilutions. Wells were then incubated with an anti-human IgG antibody conjugated to HRP, that was developed using TMB reagent. Absorbance was read at 630 nm.

FIG. 4B shows results of further experiments assessing CT1 human serum binding and neutralization. To determine the effect of neutralizing antibodies on DT or CT1's capacity to intoxicate cells, various DT/CT1 toxin chimeras (indicated) were incubated with human sera (or PBS) and then added to Vero cells, and protein synthesis levels were measured. EC50 values were calculated and the fold-difference from PBS controls was plotted. Human sera had no effect on the ability of CT1 to intoxicate cells, demonstrating that it is not neutralized by human sera.

FIG. 5 is a schematic of key residues conserved between DT an CT1.

FIGS. 6A and 6B depict results of functional characterization of translocases.

FIG. 7A shows that antibodies in human sera recognize DT, but show no binding to CT1.

FIG. 7B shows that antibodies in human sera recognize the translocase from DT, but not CT1.

FIG. 8 shows that anti-DT antibodies do not neutralize CT1-based immunotoxins.

FIG. 9 shows a phylogenetic tree illustrating the relationship of various translocases.

### DETAILED DESCRIPTION

Generally, the present disclosure provides novel bacterial translocation domains for use in cellular delivery. Recombinant polypeptides comprising these translocation domains are described. The recombinant polypeptides are intended for use in delivery of cargo molecules, including therapeutic polypeptides.

### Recombinant Polypeptides

In one aspect, there is provided a recombinant polypeptide of general formula (I):

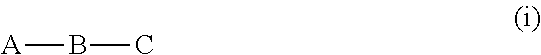

wherein:
A is a cargo molecule;
B is a translocation polypeptide comprising:
a) a translocation domain from:
the Austwickia chelonae protein of SEQ ID NO: 2,
the Streptosporangium nondiastaticum protein of GenBank Accession PSJ28985.1,
the Streptomyces sp. TLI 053 protein of GenBank Accession SDT83331.1,
the Streptomyces sp. SLBN-118 protein of GenBank Accession WP_160159328.1,
the Streptomyces sp. AA8 protein of GenBank Accession WP_168096531.1,
the Streptomyces roseoverticillatus protein of GenBank Accession WP_078659863.1,
the Streptomyces piniterrae protein of GenBank Accession JZ58907.1,
the Streptomyces MBT76 protein of GenBank Accession WP_0791103f21.1,
the Streptomyces klenkii protein of GenBank Accession WP_120757473.1,
the Streptomyces albireticuli protein of GenBank Accession WP_095582082.1,
the Streptacidiphilus pinicola protein of GenBank Accession WP_133259917.1,
the Seinonella peptonophila protein of GenBank Accession WP_073156187.1,
the Longimycelium tulufanense protein of GenBank Accession WP_189053160.1,
the Austwickia sp. TVS 96-490-7B protein of GenBank Accession WP_219106995.1,
the Austwickia chelonae LK16-18 protein of GenBank Accession WP_162873017.1;
the Klebsiella aerogenes protein of GenBank Accession EIZ2913133.1,
the Streptomyces sp. MUM 178J protein of GenBank Accession MCH0551590.1,
the Crossiella cryophila protein of GenBank Accession MBB4677777.1,
the Allokutzneria sp. NRRL B-24872 protein of GenBank Accession WP_143261759.1,
the Allokutzneria albata protein of GenBank Accession WP_156051914.1,
the Streptomyces sp. AV19 protein of GenBank Accession WP_199893204.1,
the Streptomyces sp. NRBC_110611 protein of GenBank Accession WP_147264604.1,
the Streptomyces syringium protein of GenBank Accession WP_209513619.1,
the Pseudonocardiaceae bacterium YIM PH 21723 protein of GenBank Accession RJQ69589.1,
the Actinokineospora bangkokensis protein of GenBank Accession WP_143218892.1,
the Streptomyces eurocidicus protein of GenBank Accession MBF6055834.1,
the Streptomyces pathocidini protein of GenBank Accession WP_169790908.1, or
the Streptomyces caatingaensis protein of GenBank Accession WP_157868472.1, or
b) a translocation domain that is at least 80% identical to the translocation
domain defined in a); and
C is a targeting moiety.

In one embodiment, a), the translocation domain is from:
the Austwickia chelonae protein of SEQ ID NO: 2,
the Streptosporangium nondiastaticum protein of GenBank Accession PSJ28985.1,
the Streptomyces sp. TLI 053 protein of GenBank Accession SDT83331.1,
the Streptomyces sp. SLBN-118 protein of GenBank Accession WP_160159328.1,
the Streptomyces sp. AA8 protein of GenBank Accession WP_168096531.1,
the Streptomyces roseoverticillatus protein of GenBank Accession WP_078659863.1,
the Streptomyces piniterrae protein of GenBank Accession JZ58907.1,
the Streptomyces MBT76 protein of GenBank Accession WP_079110321.1,
the Streptomyces klenkii protein of GenBank Accession WP_120757473.1,
the Streptacidiphilus pinicola protein of GenBank Accession WP_133259917.1,
the Longimycelium tulufanense protein of GenBank Accession WP_189053160.1,
the Austwickia sp. TVS 96-490-7B protein of GenBank Accession WP_219106995.1, or
the Austwickia chelonae LK16-18 protein of GenBank Accession WP_162873017.1.

In one embodiment, in a), the translocation domain is from:
the Austwickia chelonae protein of SEQ ID NO: 2,
the Streptomyces sp. TLI 053 protein of GenBank Accession SDT83331.1,
the Streptomyces klenkii protein of GenBank Accession WP_120757473.1,
the Austwickia sp. TVS 96-490-7B protein of GenBank Accession WP_219106995.1, or
the Austwickia chelonae LK16-18 protein of GenBank Accession WP_162873017.1.

In one embodiment, in a), the translocation domain is from:
the Austwickia chelonae protein of SEQ ID NO: 2,
the Austwickia chelonae LK16-18 protein of GenBank Accession WP_162873017.1.

In one embodiment, in a):
i. the translocation domain from the Austwickia chelonae protein herein named CT1 has the amino acid sequence of SEQ ID NO: 3,
ii. the translocation domain from the Streptosporangium nondiastaticum protein of GenBank Accession PSJ28985.1 has the amino acid sequence of SEQ ID NO: 4,
iii. the translocation domain from the Streptomyces sp. TLI 053 protein of GenBank Accession SDT83331.1 has the amino acid sequence of SEQ ID NO: 5,
iv. the translocation domain from the Streptomyces sp. SLBN-118 protein of GenBank Accession WP_160159328.1 has the amino acid sequence of SEQ ID NO: 6,
v. the translocation domain from the Streptomyces sp. AA8 protein of GenBank Accession WP_168096531.1 has the amino acid sequence of SEQ ID NO: 7,
vi. the translocation domain from the Streptomyces roseoverticillatus protein of GenBank Accession WP_078659863.1 has the amino acid sequence of SEQ ID NO: 8,
vii. the translocation domain from the Streptomyces piniterrae protein of GenBank Accession JZ58907.1 has the amino acid sequence of SEQ ID NO: 9,
viii. the translocation domain from the Streptomyces MBT76 protein of GenBank Accession WP_079110321.1 has the amino acid sequence of SEQ ID NO: 10,
ix. the translocation domain from the Streptomyces klenkii protein of GenBank Accession WP_120757473.1 has the amino acid sequence of SEQ ID NO: 11,
x. the translocation domain from the Streptomyces albireticuli protein of GenBank Accession WP_095582082.1 has the amino acid sequence of SEQ ID NO: 12,
xi. the translocation domain from the Streptacidiphilus pinicola protein of GenBank Accession WP_133259917.1 has the amino acid sequence of SEQ ID NO: 13,
xii. the translocation domain from the Seinonella peptonophila protein of GenBank Accession WP_073156187.1 has the amino acid sequence of SEQ ID NO: 14,
xiii. the translocation domain from the Longimycelium tulufanense protein of GenBank Accession WP_189053160.1 has the amino acid sequence of SEQ ID NO: 15,
xiv. the translocation domain from the Austwickia sp. TVS 96-490-7B protein of GenBank Accession WP_219106995.1 has the amino acid sequence of SEQ ID NO: 16,
xv. the translocation domain from the Austwickia chelonae LK16-18 protein of GenBank Accession WP_162873017.1 has the amino acid sequence of SEQ ID NO: 17, and
xvi. the translocation domain has the amino acid sequence of any one of SEQ ID Nos: 36 to 48.

In one embodiment, in a):
i. the translocation domain from the Austwickia chelonae protein herein named CT1 has the amino acid sequence of SEQ ID NO: 3,
ii. the translocation domain from the Streptosporangium nondiastaticum protein of GenBank Accession PSJ28985.1 has the amino acid sequence of SEQ ID NO: 4,
iii. the translocation domain from the Streptomyces sp. TLI 053 protein of GenBank Accession SDT83331.1 has the amino acid sequence of SEQ ID NO: 5,
iv. the translocation domain from the Streptomyces sp. SLBN-118 protein of GenBank Accession WP_160159328.1 has the amino acid sequence of SEQ ID NO: 6,
v. the translocation domain from the Streptomyces sp. AA8 protein of GenBank Accession WP_168096531.1 has the amino acid sequence of SEQ ID NO: 7,
vi. the translocation domain from the Streptomyces roseoverticillatus protein of GenBank Accession WP_078659863.1 has the amino acid sequence of SEQ ID NO: 8,
vii. the translocation domain from the Streptomyces piniterrae protein of GenBank Accession JZ58907.1 has the amino acid sequence of SEQ ID NO: 9,
viii. the translocation domain from the Streptomyces MBT76 protein of GenBank Accession WP_079110321.1 has the amino acid sequence of SEQ ID NO: 10,
ix. the translocation domain from the Streptomyces klenkii protein of GenBank Accession WP_120757473.1 has the amino acid sequence of SEQ ID NO: 11,
xi. the translocation domain from the Streptacidiphilus pinicola protein of GenBank Accession WP_133259917.1 has the amino acid sequence of SEQ ID NO: 13,
xiii. the translocation domain from the Longimycelium tulufanense protein of GenBank Accession WP_189053160.1 has the amino acid sequence of SEQ ID NO: 15,
xiv. the translocation domain from the Austwickia sp. TVS 96-490-7B protein of GenBank Accession WP_219106995.1 has the amino acid sequence of SEQ ID NO: 16, and
xv. the translocation domain from the Austwickia chelonae LK16-18 protein of GenBank Accession WP_162873017.1 has the amino acid sequence of SEQ ID NO: 17.

A “translocation polypeptide”, as referred to herein, is intended to refer to a polypeptide that comprises a translocation domain.

A “translocation domain” as referred to herein is a polypeptide sequence that functions to facilitate transport the protein in which it occurs across a cell membrane, thereby facilitating cell entry. This activity can be assessed, for example, using the assays described herein.

In one embodiment, the translocation polypeptide comprises:
a) the translocation domain from the Austwickia chelonae protein, CT1, wherein the translocation domain has amino acid sequence of SEQ ID NO: 3 (herein termed “CT1-T”), or
b) a translocation domain that is at least 80% identical to the translocation domain defined in a).

In one embodiment, the translocation polypeptide comprises:
a) the translocation domain from the Streptosporangium nondiastaticum protein of GenBank Accession PSJ28985.1, wherein the translocation domain has the amino acid sequence of SEQ ID NO: 4, or
b) a translocation domain that is at least 80% identical to the translocation domain defined in a).

In one embodiment, the translocation polypeptide comprises:
a) the translocation domain from the Streptomyces sp. TLI 053 protein of GenBank Accession SDT83331.1, wherein the translocation domain has the amino acid sequence of SEQ ID NO: 5, or
b) a translocation domain that is at least 80% identical to the translocation domain defined in a).

In one embodiment, the translocation polypeptide comprises:
a) the translocation domain from the Streptomyces sp. SLBN-118 protein of GenBank Accession WP_160159328.1, wherein the translocation domain has the amino acid sequence of SEQ ID NO: 6, or
b) a translocation domain that is at least 80% identical to the translocation domain defined in a).

In one embodiment, the translocation polypeptide comprises:
a) the translocation domain from the Streptomyces sp. AA8 protein of GenBank Accession WP_168096531.1, wherein the translocation domain has the amino acid sequence of SEQ ID NO: 7, or
b) a translocation domain that is at least 80% identical to the translocation domain defined in a).

In one embodiment, the translocation polypeptide comprises:
a) the translocation domain from the Streptomyces roseoverticillatus protein of GenBank Accession WP_078659863.1, wherein the translocation domain has the amino acid sequence of SEQ ID NO: 8, or
b) a translocation domain that is at least 80% identical to the translocation domain defined in a).

In one embodiment, the translocation polypeptide comprises:
a) the translocation domain from the Streptomyces piniterrae protein of GenBank Accession JZ58907.1, wherein the translocation domain has the amino acid sequence of SEQ ID NO: 9, or
b) a translocation domain that is at least 80% identical to the translocation domain defined in a).

In one embodiment, the translocation polypeptide comprises:
a) the translocation domain from the Streptomyces MBT76 protein of GenBank Accession WP_079110321.1, wherein the translocation domain has the amino acid sequence of SEQ ID NO: 10, or
b) a translocation domain that is at least 80% identical to the translocation domain defined in a).

In one embodiment, the translocation polypeptide comprises:
a) the translocation domain from the Streptomyces klenkii protein of GenBank Accession WP_120757473.1, wherein the translocation domain has the amino acid sequence of SEQ ID NO: 11, or
b) a translocation domain that is at least 80% identical to the translocation domain defined in a).

In one embodiment, the translocation polypeptide comprises:
a) the translocation domain from the Streptomyces albireticuli protein of GenBank Accession WP_095582082.1, wherein the translocation domain has the amino acid sequence of SEQ ID NO: 12, or
b) a translocation domain that is at least 80% identical to the translocation domain defined in a).

In one embodiment, the translocation polypeptide comprises:
a) the translocation domain from the Streptacidiphilus pinicola protein of GenBank Accession WP_133259917.1, wherein the translocation domain has the amino acid sequence of SEQ ID NO: 13, or
b) a translocation domain that is at least 80% identical to the translocation domain defined in a).

In one embodiment, the translocation polypeptide comprises:
a) the translocation domain from the Seinonella peptonophila protein of GenBank Accession WP_073156187.1, wherein the translocation domain has the amino acid sequence of SEQ ID NO: 14, or
b) a translocation domain that is at least 80% identical to the translocation domain defined in a).

the translocation polypeptide comprises:
a) the translocation domain from the Longimycelium tulufanense protein of GenBank Accession WP_189053160.1, wherein the translocation domain has the amino acid sequence of SEQ ID NO: 15, or
b) a translocation domain that is at least 80% identical to the translocation domain defined in a).

In one embodiment, the translocation polypeptide comprises:
a) the translocation domain from the Austwickia sp. TVS 96-490-7B protein of GenBank Accession WP_219106995.1, wherein the translocation domain has the amino acid sequence of SEQ ID NO: 16, or
b) a translocation domain that is at least 80% identical to the translocation domain defined in a).

In one embodiment, the translocation polypeptide comprises:
a) the translocation domain from the Austwickia chelonae LK16-18 protein of GenBank Accession WP_162873017.1, wherein the translocation domain has the amino acid sequence of SEQ ID NO: 17, or
b) a translocation domain that is at least 80% identical to the translocation domain defined in a).

In one embodiment, the translocation polypeptide comprises:
a) the translocation domain from the Klebsiella aerogenes protein of GenBank Accession EIZ2913133.1, wherein the translocation domain has the amino acid sequence of SEQ ID NO: 36, or
b) a translocation domain that is at least 80% identical to the translocation domain defined in a).

In one embodiment, the translocation polypeptide comprises:
a) the translocation domain from the Streptomyces sp. MUM 178J protein of GenBank Accession MCH0551590.1, wherein the translocation domain has the amino acid sequence of SEQ ID NO: 37, or
b) a translocation domain that is at least 80% identical to the translocation domain defined in a).

In one embodiment, the translocation polypeptide comprises:
a) the translocation domain from the Crossiella cryophila protein of GenBank Accession MBB4677777.1, wherein the translocation domain has the amino acid sequence of SEQ ID NO: 38, or
b) a translocation domain that is at least 80% identical to the translocation domain defined in a).

In one embodiment, the translocation polypeptide comprises:
a) the translocation domain from the Allokutzneria sp. NRRL B-24872 protein of GenBank Accession WP_143261759.1, wherein the translocation domain has the amino acid sequence of SEQ ID NO: 39, or
b) a translocation domain that is at least 80% identical to the translocation domain defined in a).

In one embodiment, the translocation polypeptide comprises:
a) the translocation domain from the Allokutzneria albata protein of GenBank Accession WP_156051914.1, wherein the translocation domain has the amino acid sequence of SEQ ID NO: 40, or
b) a translocation domain that is at least 80% identical to the translocation domain defined in a).

In one embodiment, the translocation polypeptide comprises:
a) the translocation domain from the Streptomyces sp. AV19 protein of GenBank Accession WP_199893204.1, wherein the translocation domain has the amino acid sequence of SEQ ID NO: 41, or
b) a translocation domain that is at least 80% identical to the translocation domain defined in a).

In one embodiment, the translocation polypeptide comprises:
a) the translocation domain from the Streptomyces sp. NRBC_110611 protein of GenBank Accession WP_147264604.1, wherein the translocation domain has the amino acid sequence of SEQ ID NO: 42, or
b) a translocation domain that is at least 80% identical to the translocation domain defined in a).

In one embodiment, the translocation polypeptide comprises:
a) the translocation domain from the Streptomyces syringium protein of GenBank Accession WP_209513619.1, wherein the translocation domain has the amino acid sequence of SEQ ID NO: 43, or
b) a translocation domain that is at least 80% identical to the translocation domain defined in a).

In one embodiment, the translocation polypeptide comprises:
a) the translocation domain from the Pseudonocardiaceae bacterium YIM PH 21723 protein of GenBank Accession RJQ69589.1, wherein the translocation domain has the amino acid sequence of SEQ ID NO: 44, or
b) a translocation domain that is at least 80% identical to the translocation domain defined in a).

In one embodiment, the translocation polypeptide comprises:
a) the translocation domain from the Actinokineospora bangkokensis protein of GenBank Accession WP_143218892.1, wherein the translocation domain has the amino acid sequence of SEQ ID NO: 45, or
b) a translocation domain that is at least 80% identical to the translocation domain defined in a).

In one embodiment, the translocation polypeptide comprises:
a) the translocation domain from the Streptomyces eurocidicus protein of GenBank Accession MBF6055834.1, wherein the translocation domain has the amino acid sequence of SEQ ID NO: 46, or
b) a translocation domain that is at least 80% identical to the translocation domain defined in a).

In one embodiment, the translocation polypeptide comprises:
a) the translocation domain from the Streptomyces pathocidini protein of GenBank Accession WP_169790908.1, wherein the translocation domain has the amino acid sequence of SEQ ID NO: 47, or
b) a translocation domain that is at least 80% identical to the translocation domain defined in a).

In one embodiment, the translocation polypeptide comprises:
a) the translocation domain from the Streptomyces caatingaensis protein of GenBank Accession WP_157868472.1, wherein the translocation domain has the amino acid sequence of SEQ ID NO: 48, or
b) a translocation domain that is at least 80% identical to the translocation domain defined in a).

The Austwickia chelonae LK16-18 protein of GenBank Accession WP_162873017.1 may be referred to herein as “CT2”. The Austwickia sp. TVS 96-490-7B protein of GenBank Accession WP_219106995.1 may be referred to herein as “CT3”.

In one embodiment, the translocation domain is as defined in b) in any one of the above embodiments and is at least at least 85% identical to the translocation domain defined in a) across the full length thereof. In one embodiment, the translocation domain is as defined in b) in any one of the above embodiments and is at least at least 90% identical to the translocation domain defined in a) across the full length thereof. In one embodiment, the translocation domain is as defined in b) in any one of the above embodiments and is at least at least 95% identical to the translocation domain defined in a) across the full length thereof. In one embodiment, the translocation domain is as defined in b) in any one of the above embodiments and is at least at least 98% identical to the translocation domain defined in a) across the full length thereof. In one embodiment, the translocation domain is as defined in b) in any one of the above embodiments and is at least at least 99% identical to the translocation domain defined in a) across the full length thereof.

In one embodiment, the translocation polypeptide is as defined in a) in any one of the above embodiments.

In one embodiment, the translocation polypeptide comprises:
a) the translocation domain from the Austwickia chelonae CT1 protein, wherein the translocation domain has amino acid sequence of SEQ ID NO: 3.

In one embodiment, the translocation polypeptide comprises:
a) the translocation domain from the Streptosporangium nondiastaticum protein of GenBank Accession PSJ28985.1, wherein the translocation domain has the amino acid sequence of SEQ ID NO: 4.

In one embodiment, the translocation polypeptide comprises:
a) the translocation domain from the Streptomyces sp. TLI 053 protein of GenBank Accession SDT83331.1, wherein the translocation domain has the amino acid sequence of SEQ ID NO: 5.

In one embodiment, the translocation polypeptide comprises:
a) the translocation domain from the Streptomyces sp. SLBN-118 protein of GenBank Accession WP_160159328.1, wherein the translocation domain has the amino acid sequence of SEQ ID NO: 6.

In one embodiment, the translocation polypeptide comprises:
a) the translocation domain from the Streptomyces sp. AA8 protein of GenBank Accession WP_168096531.1, wherein the translocation domain has the amino acid sequence of SEQ ID NO: 7.

In one embodiment, the translocation polypeptide comprises:
a) the translocation domain from the Streptomyces roseoverticillatus protein of GenBank Accession WP_078659863.1.

In one embodiment, the translocation polypeptide comprises:
a) the translocation domain from the Streptomyces piniterrae protein of GenBank Accession JZ58907.1.

In one embodiment, the translocation polypeptide comprises:
a) the translocation domain from the Streptomyces MBT76 protein of GenBank Accession WP_079110321.1, wherein the translocation domain has the amino acid sequence of SEQ ID NO: 10.

In one embodiment, the translocation polypeptide comprises:
a) the translocation domain from the Streptomyces klenkii protein of GenBank Accession WP_120757473.1.

In one embodiment, the translocation polypeptide comprises:
a) the translocation domain from the Streptomyces albireticuli protein of GenBank Accession WP_095582082.1, wherein the translocation domain has the amino acid sequence of SEQ ID NO: 12.

In one embodiment, the translocation polypeptide comprises:
a) the translocation domain from the Streptacidiphilus pinicola protein of GenBank Accession WP_133259917.1, wherein the translocation domain has the amino acid sequence of SEQ ID NO: 13.

In one embodiment, the translocation polypeptide comprises:
a) the translocation domain from the Seinonella peptonophila protein of GenBank Accession WP_073156187.1, wherein the translocation domain has the amino acid sequence of SEQ ID NO: 14.
the translocation polypeptide comprises:
a) the translocation domain from the Longimycelium tulufanense protein of GenBank Accession WP_189053160.1, wherein the translocation domain has the amino acid sequence of SEQ ID NO: 15.

In one embodiment, the translocation polypeptide comprises:
a) the translocation domain from the Austwickia sp. TVS 96-490-7B protein of GenBank Accession WP_219106995.1, wherein the translocation domain has the amino acid sequence of SEQ ID NO: 16.

In one embodiment, the translocation polypeptide comprises:
a) the translocation domain from the Austwickia chelonae LK16-18 protein of GenBank Accession WP_162873017.1, wherein the translocation domain has the amino acid sequence of SEQ ID NO: 17.

In some embodiments, the translocation domains having the amino acid sequences of SEQ ID NO: 12 (from the Streptomyces albireticuli protein of GenBank Accession WP_095582082.1) and 14 (from the Seinonella peptonophila protein of GenBank Accession WP_073156187.1) are excluded from the above-described embodiments.

In some embodiments, the translocation polypeptide may comprise functional truncations of the full-length protein that comprise any one of the above-describe corresponding translocation domains, wherein the function of the translocation domain is maintained.

In one embodiment, A and B are separated by a linker. In one embodiment, A and B are separated by an amino acid linker. In one embodiment, the amino acid linker comprises (G₄S)₂. In one embodiment, the linker is cleavable. In one embodiment, the linker comprises a protease recognition site. In one embodiment, the protease recognition site isa furin protease recognition site. In one embodiment, the protease recognition site is bracketed by cysteine residues to allow for disulphide bond formation to form an intramolecular loop. In one embodiment, the amino acid linker comprises a furin protease recognition site bracketed by cysteine residues. In one example embodiment, the amino acid linker comprises SEQ ID NO: 32, which comprises the furin protease recognition site and bracketing cysteine residues. In one embodiment, said (G₄S)₂ is positioned N-terminally with respect to said SEQ ID NO: 32. In one embodiment, the linker is self-cleaving. In one embodiment, the linker is self-clearing. In one embodiment, the linker comprises an autoprocessing domain.

In one embodiment, B and C are separated by a linker. In one embodiment, B and C are separated by an amino acid linker. In one embodiment, the amino acid linker comprises (G4S)2. In one embodiment, the amino acid linker comprises (G4S)2. In one embodiment, the amino acid linker comprises SEQ ID NO: 33. In one embodiment, wherein said (G4S)2 is positioned N-terminally with respect to said SEQ ID NO: 33.

In one embodiment, the recombinant polypeptide comprises an amino acid sequence that is at least 80% identical to amino acids 1 to 822 of SEQ ID NO: 23, preferably 90% identical to amino acids 1 to 822 of SEQ ID NO: 23, more preferably 95% identical to amino acids 1 to 822 of SEQ ID NO: 23, even more preferably 100% identical to amino acids 1 to 822 of SEQ ID NO: 23.

In one embodiment, the recombinant polypeptide comprises the sequence of SEQ ID NO: 23.

In one embodiment, the recombinant polypeptide comprises an amino acid sequence that is at least 80% identical to amino acids 1 to 822 SEQ ID NO: 24, preferably 90% identical to amino acids 1 to 822 SEQ ID NO: 24, more preferably 95% identical to amino acids 1 to 822 SEQ ID NO: 24, even more preferably 100% identical to amino acids 1 to 822 SEQ ID NO: 24.

In one embodiment, the recombinant polypeptide comprises the sequence of SEQ ID NO: 24.

In one embodiment, the recombinant polypeptide comprises an amino acid sequence that is at least 80% identical to amino acids 1 to 821 of SEQ ID NO: 25, preferably 90% identical to amino acids 1 to 821 of SEQ ID NO: 25, more preferably 95% identical to amino acids 1 to 821 of SEQ ID NO: 25, even more preferably 100% identical to amino acids 1 to 821 of SEQ ID NO: 25.

In one embodiment, the recombinant polypeptide comprises the sequence of SEQ ID NO: 25.

In one embodiment, the recombinant polypeptide comprises an amino acid sequence that is at least 80% identical to amino acids 1 to 806 of SEQ ID NO: 26, preferably 90% identical to amino acids 1 to 806 of SEQ ID NO: 26, more preferably 95% identical to amino acids 1 to 806 of SEQ ID NO: 26, even more preferably 100% identical to amino acids 1 to 806 of SEQ ID NO: 26.

In one embodiment, the recombinant polypeptide comprises the sequence of SEQ ID NO: 26.

In one embodiment, the recombinant polypeptide comprises an amino acid sequence that is at least 80% identical to amino acids 1 to 811 of SEQ ID NO: 27, preferably 90% identical to amino acids 1 to 811 of SEQ ID NO: 27, more preferably 95% identical to amino acids 1 to 811 of SEQ ID NO: 27, even more preferably 100% identical to amino acids 1 to 811 of SEQ ID NO: 27.

In one embodiment, the recombinant polypeptide comprises the sequence of SEQ ID NO: 27.

In one embodiment, the recombinant polypeptide comprises an amino acid sequence that is at least 80% identical to amino acids 1 to 822 of SEQ ID NO: 28, preferably 90% identical to amino acids 1 to 822 of SEQ ID NO: 28, more preferably 95% identical to amino acids 1 to 822 of SEQ ID NO: 28, even more preferably 100% identical to amino acids 1 to 822 of SEQ ID NO: 28.

In one embodiment, the recombinant polypeptide comprises the sequence of SEQ ID NO: 28.

In one embodiment, the recombinant polypeptide comprises an amino acid sequence that is at least 80% identical to amino acids 1 to 796 of SEQ ID NO: 29, preferably 90% identical to amino acids 1 to 796 of SEQ ID NO: 29, more preferably 95% identical to amino acids 1 to 796 of SEQ ID NO: 29, even more preferably 100% identical to amino acids 1 to 796 of SEQ ID NO: 29.

In one embodiment, the recombinant polypeptide comprises the sequence of SEQ ID NO: 29.

In one embodiment, the recombinant polypeptide comprises an amino acid sequence that is at least 80% identical to amino acids 1 to 822 of SEQ ID NO: 30, preferably 90% identical to amino acids 1 to 822 of SEQ ID NO: 30, more preferably 95% identical to amino acids 1 to 822 of SEQ ID NO: 30, even more preferably 100% identical to amino acids 1 to 822 of SEQ ID NO: 30.

In one embodiment, the recombinant polypeptide comprises the sequence of SEQ ID NO: 30.

In one embodiment, the targeting moiety comprises a targeting polypeptide or aptamer.

In one embodiment, the targeting polypeptide comprises an antibody, a binding fragment of an antibody, an affibody, an affitin, a DARPin, or a receptor ligand.

In one embodiment, the targeting polypeptide comprises an affibody against Her3. In one embodiment, the affibody against Her3 comprises the amino acid sequence of SEQ ID NO: 19.

In one embodiment, targeting polypeptide comprises a receptor ligand for αvβ6 integrin. In one embodiment, the receptor ligand for αvβ6 integrin comprises the amino acid sequence of SEQ ID NO: 20.

In one embodiment, the targeting moiety comprises at least two targeting polypeptides, at least two aptamers, or a combination of a targeting polypeptide and an aptamer. In one embodiment, the at least two targeting polypeptides are selected from the group an antibody, a binding fragment of an antibody, an affibody, a peptide, an affitin, a DARPin, a receptor ligand, and combinations thereof. In one embodiment, the at least two targeting polypeptides comprise an affibody against Her3. In one embodiment, the affibody against Her3 comprises the amino acid sequence of SEQ ID NO: 19. In one embodiment, the at least two targeting polypeptides comprise a receptor ligand for αvβ6 integrin. In one embodiment, the receptor ligand for αvβ6 integrin comprises the amino acid sequence of SEQ ID NO: 20. In one embodiment, the at least two targeting polypeptides comprise both the affibody against Her3 and the receptor ligand for αvβ6 integrin, preferably wherein the former comprises the amino acid sequence of SEQ ID NO: 19 and the latter comprises the amino acid sequence of SEQ ID NO: 20. In one embodiment, the at least two least two targeting polypeptides, the at least two aptamers, or the combination are separated by an amino acid linker. In one embodiment, the amino acid linker comprises (G₄S)₂.

In one embodiment, the targeting moiety binds to a cell surface protein.

In one embodiment, the cell surface protein is lineage-specific or tissue-specific.

In one embodiment, the cell surface protein is ubiquitously expressed.

In one embodiment, the cell surface protein is expressed in a disease cell.

In one embodiment, the cell surface protein is specific to a disease cell and is not expressed in a corresponding healthy cell.

In one embodiment, the cell surface protein has elevated expression in a disease cell compared to a corresponding healthy cell.

In one embodiment, the disease cell is a cancer cell.

In one embodiment, the cargo molecule comprises a cargo polypeptide.

The cargo polypeptide may comprise any polypeptide for which cellular delivery is desired. The cargo polypeptide may comprise an enzyme, or an active fragment thereof having substantially the same activity. By ‘substantially the same activity’ is meant that a core function of the enzyme is substantially unaltered in the fragment.

The cargo polypeptide may have a molecular weight of less than 10 kDa, greater than 10 kDa, greater than 20 kDa, greater than 30 kDa, greater than 50 kDa, greater than 100 kDa, or greater than 150 kDa.

The cargo polypeptide comprises a genome-modifying protein. The genome-modifying protein comprises a zinc finger nuclease (ZFN), a transcription activator-like effector nuclease (TALEN), or a CRISPR (clustered regularly interspaced short palindromic repeat) protein. The CRISPR protein may be Cas9. The cargo polypeptide may comprise a complex of the genome-modifying protein and a nucleic acid, such as a guide nucleic acid. For instance, Cas9 may be complexed with a nucleic acid (such as a guide RNA), such as crRNA, trRNA, and/or sgRNA.

In one embodiment, the cargo molecule comprises a therapeutic polypeptide.

By ‘therapeutic polypeptide’ is meant any protein, the cellular delivery of which could be used for a therapeutic purpose. It is well known, for example, that many human diseases or disorders are caused by or characterized by protein deficiency. Therapeutic proteins encompass proteins, the delivery of which could ameliorate or correct such a deficiency. A therapeutic protein may act to replace a protein that is deficient in the disease or disorder. A therapeutic protein may be the protein that is deficient in the disease or disorder. However, a therapeutic protein need not necessarily be identical to the protein that is deficient in the disease or disorder. For instance, a therapeutic protein may be an active fragment or modified form of a deficient protein. A therapeutic protein may also partially or fully functionally compensate for the protein deficiency underlying the disease or disorder. A therapeutic protein may also ameliorate or correct downstream or secondary effects of the cellular deficiency in a particular protein.

In one embodiment, the therapeutic polypeptide comprises a cytotoxic polypeptide, preferably a polypeptide toxin or a functional fragment thereof. In one embodiment, the cytotoxic polypeptide comprises a catalytic domain from Diphtheria Toxin. In one embodiment, the cytotoxic polypeptide comprises a catalytic domain from a Chelona Toxin, such as from CT1 (SEQ ID NO: 2), CT2 (SEQ ID NO: 21), or CT3 (SEQ ID NO: 35) as described herein.

In on embodiment, the catalytic domain is from CT1. In one embodiment, the catalytic domain from the Chelona Toxin set forth in SEQ ID NO: 2 (CT1) has the amino acid sequence according to amino acid positions 1 to 186 of SEQ ID NO: 2.

In on embodiment, the catalytic domain is from CT2. In one embodiment, the catalytic domain from the Chelona Toxin set forth in SEQ ID NO: 21 (CT2) has the amino acid sequence according to amino acid positions 1 to 186 of SEQ ID NO: 21.

In on embodiment, the catalytic domain is from CT3. In one embodiment, the catalytic domain from the Chelona Toxin set forth in SEQ ID NO: 35 (CT3) has the amino acid sequence according to amino acid positions 1 to 191 of SEQ ID NO: 35.

In one embodiment, the therapeutic polypeptide comprises a protein that is deficient is a disease state, or a functional fragment thereof.

In one embodiment, the therapeutic polypeptide comprises Ras/Rap1-specific endopeptidase (RRSP) from Vibrio vulnificus, e.g., as is set forth in SEQ ID NO: 18. In some embodiments the therapeutic polypeptide may be at least 80% identical to RRSP. In some embodiments the therapeutic polypeptide may be at least 90% identical to RRSP. In some embodiments the therapeutic polypeptide may be at least 95% identical to RRSP. In some embodiments the therapeutic polypeptide may be at least 98% identical to RRSP. These sequence variant may retain substantially the same activity as full-length RRSP.

In one embodiment, the cargo molecule comprises an N-terminal cysteine residue for use in “click” chemistry bioconjugation.

In one embodiment, the cargo molecule comprises a nucleic acid molecule.

Percent sequence identifies described herein may be calculated across the full length of an alignment.

The amino acid sequences referred to herein may encompass sequence differences, in some embodiments compared to the references sequences (such as those set forth in Table 1, below). These may be variants, mutations, insertions, or deletions. In some applications, it may be important to ensure that the primary function of the protein is not substantially altered or abrogated, but this can be readily tested, e.g. using assays described herein. The amino acid sequences described herein may comprise a sequence of 75% or greater, 80% or greater, 85% or greater, 90% or greater, 95% or greater, or 99% or greater identity to the references sequences. The amino acid sequences may encompass conservative amino substitutions. Conservative amino acid substitutions which are known in the art are as follows with conservative substitutable candidate amino acids showing in parentheses: Ala (Gly, Ser); Arg (Gly, Gln); Asn (Gln; His); Asp (Glu); Cys (Ser); Gln (Asn, Lys); Glu (Asp); Gly (Ala, Pro); His (Asn; Gln); Ile (Leu; Val); Leu (Ile; Val); Lys (Arg; Gln); Met (Leu, Ile); Phe (Met, Leu, Tyr); Ser (Thr; Gly); Thr (Ser; Val); Trp (Tyr); Tyr (Trp; Phe); Val (Ile; Leu). Some so-called ‘functional’ variants, mutations, insertions, or deletions encompass sequences in which the function is substantially the same as that of the reference sequence, e.g. from which it is derived. This can be readily tested using assays similar to those described herein.

### Nucleic Acids and Vectors

In one aspect, there is provided a nucleic acid encoding the recombinant polypeptide as defined here. In one embodiment, the nucleic acid is DNA or RNA. The RNA may be an mRNA.

A skilled person would readily appreciate there are many ways to encode the recombinant polypeptide described herein, e.g. due to degeneracy of the genetic code, all of which are encompassed in certain embodiments. Deletions, insertions, and substitutions may also be permitted if protein function remains substantially intact. For instance, nucleic acids may have 75% or greater, 80% or greater, 85% or greater, 90% or greater, 95% or greater, or 99% or greater identity to wild-type or references sequences may be encompassed. The above-noted nucleic acids could also be codon optimized depending on the organism or expression system in which it is intended to be expressed.

In one aspect, there is provided a vector comprising the nucleic acid as defined herein.

In one embodiment, there is provided a host cell comprising the nucleic acid as defined herein or the vector as defined herein. The host cell may be transformed or transfected.

### Compositions

In one aspect, there is provided a composition comprising the recombinant polypeptide as defined herein, together with an acceptable excipient, diluent, or carrier.

In aspect embodiment, there is provided a pharmaceutical composition the recombinant polypeptide as defined herein, together with a pharmaceutically acceptable excipient, diluent, or carrier.

Pharmaceutically acceptable carriers include solvents, diluents, liquid vehicles, dispersion or suspension aids, surface active agents, isotonic agents, thickening or emulsifying agents, preservatives, solid binders, or lubricants. Carriers may be selected to prolong dwell time for sustained release appropriate to the selected route of administration. Exemplary carriers include sugars such as glucose and sucrose, starches such as corn starch and potato starch, fibers such as cellulose and its derivatives, sodium carboxymethyl cellulose, ethyl cellulose, cellulose acetate, powdered tragacanth, malt, gelatin, talc, cocoa butter, suppository waxes, oils such as peanut oil, cottonseed oil, safflower oil, sesame oil, olive oil, corn oil, and soybean oil; glycols such as propylene glycol, esters such as ethyl oleate and ethyl laurate, agar, buffering agents such as magnesium hydroxide and aluminum hydroxide, alginic acid, pyrogen-free water, isotonic saline, Ringer's solution, ethyl alcohol, phosphate buffer solutions, non-toxic compatible lubricants such as sodium lauryl sulfate and magnesium stearate, coloring agents, releasing agents, coating agents, sweeteners, flavors, perfuming agents, preservatives, and antioxidants.

Compositions can be administered to subjects through any acceptable route, such as topically (as by powders, ointments, or drops), orally, rectally, mucosally, sublingually, parenterally, intracisternally, intravaginally, intraperitoneally, bucally, ocularly, or intranasally.

Liquid dosage forms for oral administration may include emulsions, microemulsions, solutions, suspensions, syrups and elixirs. Liquid dosage forms may contain inert diluents such as water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils such as cottonseed, groundnut, corn, germ, olive, castor, and sesame oils, glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Dosage forms for topical or transdermal administration of an inventive pharmaceutical composition include ointments, pastes, creams, lotions, gels, powders, solutions, sprays, inhalants, or patches. The active agent is admixed under sterile conditions with a pharmaceutically acceptable carrier and any needed preservatives or buffers as may be required.

Injectable preparations, such as sterile injectable aqueous or oleaginous suspensions may be formulated according to the known art using suitable dispersing or wetting agents and suspending agents. The sterile injectable preparation may also be a sterile injectable solution, suspension or emulsion in a non-toxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized prior to addition of spores, for example, by filtration through a bacterial-retaining filter, or by incorporating sterilizing agents in the form of sterile solid compositions which can be dissolved or dispersed in sterile water or other sterile injectable medium prior to use.

It is often desirable to slow the absorption of the agent from subcutaneous or intramuscular injection. Delayed absorption of a parenterally administered active agent may be accomplished by dissolving or suspending the agent in an oil vehicle. Injectable depot forms are made by forming microencapsule matrices of the agent in biodegradable polymers such as polylactide-polyglycolide. Depending upon the ratio of active agent to polymer and the nature of the particular polymer employed, the rate of active agent release can be controlled. Examples of other biodegradable polymers include poly(orthoesters) and poly(anhydrides). Depot injectable formulations are also prepared by entrapping the agent in liposomes or microemulsions which are compatible with body tissues.

Compositions for rectal or vaginal administration are preferably suppositories which can be prepared by mixing the active agent(s) of this invention with suitable non-irritating excipients or carriers such as cocoa butter, polyethylene glycol or a suppository wax which are solid at ambient temperature but liquid at body temperature and therefore melt in the rectum or vaginal cavity and release the active agent(s).

Solid dosage forms for oral, mucosal or sublingual administration include capsules, tablets, pills, powders, and granules. In such solid dosage forms, the active agent is mixed with at least one inert, pharmaceutically acceptable excipient or carrier such as sodium citrate or dicalcium phosphate, fillers or extenders such as starches, sucrose, glucose, mannitol, and silicic acid, binders such as, for example, carboxymethylcellulose, alginates, gelatin, polyvinylpyrrolidinone, sucrose, and acacia, humectants such as glycerol, disintegrating agents such as agar-agar, calcium carbonate, potato or tapioca starch, alginic acid, certain silicates, and sodium carbonate, solution retarding agents such as paraffin, absorption accelerators such as quaternary ammonium compounds, wetting agents such as, for example, cetyl alcohol and glycerol monostearate, absorbents such as kaolin and bentonite clay, and lubricants such as talc, calcium stearate, magnesium stearate, solid polyethylene glycols, sodium lauryl sulfate, and mixtures thereof.

Solid compositions of a similar type may also be employed as fillers in soft and hard-filled gelatin capsules using such excipients as milk sugar as well as high molecular weight polyethylene glycols and the like. The solid dosage forms of tablets, capsules, pills, and granules can be prepared with coatings and shells such as enteric coatings, release controlling coatings and other coatings well known in the pharmaceutical formulating art. In such solid dosage forms the active agent(s) may be admixed with at least one inert diluent such as sucrose or starch. Such dosage forms may also comprise, as is normal practice, additional substances other than inert diluents, such as tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active agent(s) only, or preferentially, in a certain part of the intestinal tract, optionally, in a delayed manner. Examples of embedding compositions which can be used include polymeric substances and waxes.

The therapeutically effective amount may be determined on an individual basis or on the basis of the established amount necessary. The dosage for an individual subject is chosen in view of the subject to be treated. Dosage and administration may be adjusted to provide sufficient levels of the active agent(s) or to maintain the desired effect. Factors which may be taken into account include the severity of the disease state, contact with infectious agent in the past, potential future contact; age, weight, gender of the subject, diet, time and frequency of administration, drug combinations, reaction sensitivities, and tolerance/response to therapy. Sustained release compositions might be administered less frequently than fast-acting compositions.

### Methods and Uses

In one aspect, there is provided a method of delivery a cargo molecule to a cell comprising contacting the cell with the recombinant polypeptide as defined herein.

In one aspect, there is provided a use of the recombinant polypeptide as defined herein for delivery of the cargo molecule to a cell.

In one aspect, there is provided a use of the recombinant polypeptide as defined herein for preparation of a medicament for delivery of the cargo molecule to a cell.

In one aspect, there is provided the recombinant polypeptide as defined herein for use in delivery of the cargo molecule to a cell.

In one aspect, there is provided a method treating cancer in a subject comprising administering to the subject the recombinant polypeptide as defined herein.

In one aspect, there is provided a use of the recombinant polypeptide as defined herein for treatment of cancer in a subject.

In one aspect, there is provided a use of the recombinant polypeptide as defined herein for preparation of a medicament for treatment of cancer in a subject.

In one aspect, there is provided the recombinant polypeptide as defined herein for use in treatment of cancer in a subject.

In one aspect, there is provided a method of alleviating enzyme or protein deficiency in a cell, comprising contacting a cell with the recombinant polypeptide as described herein.

In one aspect, there is provided a use of the recombinant polypeptide as described herein for alleviating enzyme or protein deficiency in a cell.

In one aspect, there is provided a use of the recombinant polypeptide as described herein for preparation of a medicament for alleviating enzyme or protein deficiency in a cell.

In one aspect, there is provided the recombinant polypeptide as described herein for use in alleviating enzyme or protein deficiency in a cell.

By ‘alleviate, as used herein, is meant that the cargo molecule corrects or at least partially ameliorates the protein or enzyme deficiency, an aspect of the deficient protein or enzyme's function, or one or more of its downstream or secondary cellular effects or consequences.

In embodiments of the aforementioned methods and uses, the cargo may be released.

### EXAMPLES

### Example 1

### Introduction

Engineered chimeric toxins has led to the emergence of novel therapeutics for challenging diseases, such as cancer. Immunotoxins are a class of biotherapeutics comprised of bacterial toxins, such as diphtheria toxin (DT), that have been repurposed into cancer-targeted therapies-both by re-targeting their receptor binding domains (RBD) to target cancer receptors, and by delivering enzyme cargo that target intracellular oncoproteins. However, global vaccination programs against diphtheria have resulted in population-level immunity against DT, and DT-based therapeutics. To circumvent the issue of pre-existing neutralizing antibodies against DT, it was investigated whether distant homologs of DT sharing little sequence identity could retain the function of DT but avoid DT-specific neutralizing antibodies. Here, a putative gene sequence from Austwickia chelonae that is only 38% identical to DT has been structurally and functionally characterized. It has been named chelona toxin 1 (CT1). The x-ray crystallography structure thereof has been solved to 2.50 Å and it was found that its structure was highly similar to DT. Using a variety of biochemical assays, a domain-by-domain analysis was undertaken to investigate the capacity of this DT-like protein to function as both a toxin and a new platform for therapeutic protein delivery. It has been demonstrated that while each domain of this novel protein can perform its respective function as a toxin, the translocase of CT1 (CT1-T) can be engineered to target non-native receptors and deliver non-native cargo into cells. Importantly, CT1 is not recognized by to pre-existing anti-DT antibodies found in human sera and is unexpectedly superior to DT at delivering cargo into cells. Chelona toxins provide novel insights into toxin biology and represents an improved platform for therapeutic protein delivery.

### Materials and Methods

Crystallization of the DT-like protein from A. chelonae.

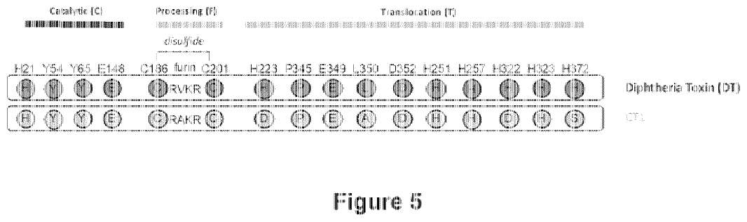

The closest DT-like protein from the species Austwickia chelonae (herein referred to as CT1 for “chelona toxin 1”) (SEQ ID NO: 2) was chosen as the candidate for an alternative immunotoxin scaffold, due to the conservation of key residues identified to be important for DT functionality (FIG. 5).

SEQ ID NO: 2 is derived from a combination of two ORFs (see GenBank Accession Nos. WP_143115263.1 and WP_040322835.1) representing two fragments of a toxin. When compared to the genomic sequence of Austwickia chelonae (see GenBank Accession No. NZ_BAGZ01000024.1), it appeared that a 1 base pair (bp) frameshift in the genomic sequence had caused a full-length toxin to be separated into the 2 ORFs. The reading frame was restored by deleting 1 bp (NZ_BAGZ01000024.1 C41398), and the result was a full-length toxin was subsequently called “chelona toxin 1 (CT1)” (SEQ ID NO: 2). It is unclear whether the 1 bp insertion was a sequence error or reflective of a genuine mutation in Austwickia chelonae. In any case, the 1 bp insertion was removed to produce the protein and translocation domain use for the experiments described herein.

To determine the structure of CT1, the E. coli codon optimized gBlock gene fragment was ordered from Integrated DNA Technologies and cloned into the Champion™ pET SUMO E. coli expression system by Gibson Assembly.

A 50 mL starter culture of NiCo21 (DE3) E. coli cells (New England Biolabs) were inoculated into 1 L of LB medium and induced with 0.1 mM IPTG at 18 C for 18 hours. Cells were centrifuged at 5000 rpm and resuspended in lysis buffer (1% protease inhibitor cocktail, 1 mg/mL lysozyme, 0.01% Pierce™ universal nuclease inhibitor, 20 mM imidazole, 500 mM NaCl, 20 mM Tris-HCl pH 7.5). Cells were lysed with three passes through an Emulsiflex C3 (Avestin) at 15000 psi. Whole cell lysate was centrifuged at 18000×g and the supernatant was filtered through a 0.45 μm filter and passed over a HisTrap FF crude column (Cytiva). The protein was eluted with 50-75 mM imidazole, buffer exchanged into 150 mM NaCl, 20 mM Tris-HCl PH 7.5, and incubated with SUMO protease overnight at 4 C, to cleave the 6×His-SUMO affinity tag. The protein was flowed over a HisTrap FF crude column and the flowthrough (protein) was collected and concentrated to 8 mg/mL by centrifugation.

Hanging drop vapour diffusion was used to grow crystals. The condition in which CT1 crystals were obtained contained 2 μL of mother liquor (0.2M calcium chloride, 0.1M Tris-HCl pH 8.5, 25% (w/v) PEG4000) and 1 μL of 8 mg/mL protein. The drop was dehydrated over 130 μL of 2M (NH₄)₂PO₄ for 45 minutes prior to freezing in liquid nitrogen. Data was collected at the Advanced Photon Source on the 23-ID-D beamline.

Initial phases were determined using Phaser in the Phenix software package by using a multi-component search models with individual DT domains (C-domain residues 13-167, R-domain residues 391-531, T-domain residues 205-378) in which disordered loops had been removed. The structure was refined using iterative cycles of phenix.refine and autobuild.

### Protein Synthesis Assay

Vero-nLucP cells (a nanoluciferase reporter strain of Vero cells) were plated at 5000 cells/well in 96-well white clear bottom plates (Corning). The following day, protein toxin was added and incubated for 24 hours, after which cells were read for luminescence signal using the NanoGlo® Luciferase Assay kit (Promega), on a SpectraMax M5e plate reader (Molecular Devices). Data was corrected to untreated cells (100% nanoluciferase signal).

### Liposomal Dye Release Assay

Unilamellar liposomes (DOPC, 0.8% DGS-NTA, Avanti Polar Lipids) were prepared as previously described. Briefly, 1,2-dioleoyl-sn-glycero-3-phosphocholine (DOPC) (Avanti Polar Lipids) was combined with 0.8% 1,2-dioleoyl-sn-glycero-3-[(N-(5-amino-1-carboxypentyl)iminodiacetic acid) succinyl] (nickel salt) (DGS-NTA [Ni]) (Avanti Polar Lipids), dried with N₂ and 1 hour in a vacuum dessicator. Lipids were resuspended in 20 mM Tris pH 8, 35 mM 8-Hydroxypyrene-1,3,6-trisulfonic acid (HPTS), 50 mM p-xylene-bis-pyridinium bromide (DPX) (Thermo Fischer) and subject to 10× freeze-thaw cycles in dry ice and 42° C. water bath, and 10× extrusions using a 200 μm filter. The liposomes were then purified by gel filtration using a Hi Prep 16/60 Sephacryl S-300 HR column (GE Healthcare) and 150 mM NaCl, 20 mM Tris pH 8 buffer. Proteins were added in a ratio of 1:10,000 with liposomes, with a final liposome concentration of ~400 μM, in 150 mM citrate phosphate buffer ranging from pH 4.0 to 7.5, in 0.5 pH intervals. Assays were done in 96-well opaque plates (Corning), and fluorescence was monitored over a 20-minute interval, with readings being taken every 30 seconds (excitation 403 nm, emission 510 nm). Data were normalized to % of total HPTS fluorescence, by adding 0.3% Triton X-100.

### Serum Antibody Binding ELISA

Nunc MaxiSorp™ plates (Thermo Fisher Scientific) were immobilized with 2000 ng of protein after being blocked with 1% BSA, and were subsequently incubated with human serum (Pooled Human Serum frozen, Cedarlane) at various dilutions, for 1 hour. Wells were washed with PBST (0.01% tween) and then incubated with an anti-human IgG antibody conjugated to HRP (Abcam, ab102420), that was developed using TMB reagent (Thermo Fisher Scientific). Absorbance was read at 630 nm and protein wells were corrected to control wells (no-protein, +human serum).

### Serum Toxicity Assays

Protein toxins were incubated with either human serum (Pooled Human Serum frozen, Cedarlane) or mouse serum (Mouse serum sterile frozen, Cedarlane) in a 1:1 ratio, for 30 minutes at room temperature. Sample was then added to Vero-nLucP cells that had been plated to 5000 cells/well the previous day, in a 96-well white clear bottom plate (Corning). Cells were incubated for 24 hours, upon which cells were lysed and assessed for luminescence signal. Values were corrected to serum only treated cells, which represented 100% nanoluciferase signal and 100% protein synthesis.

### Results

Structural Characterization of DT-Like Protein from A. Chelonae

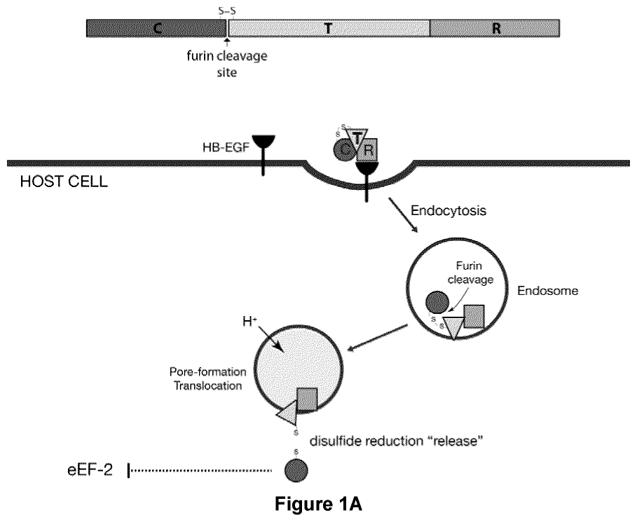

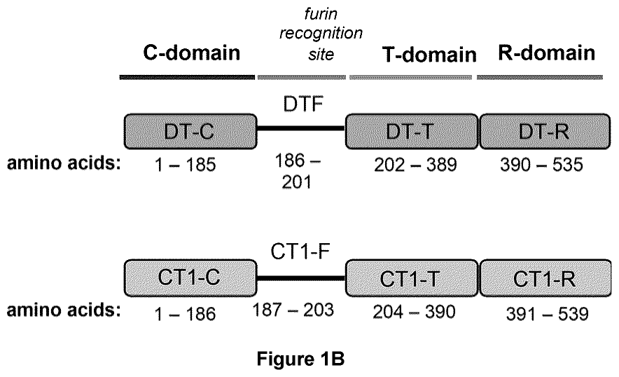

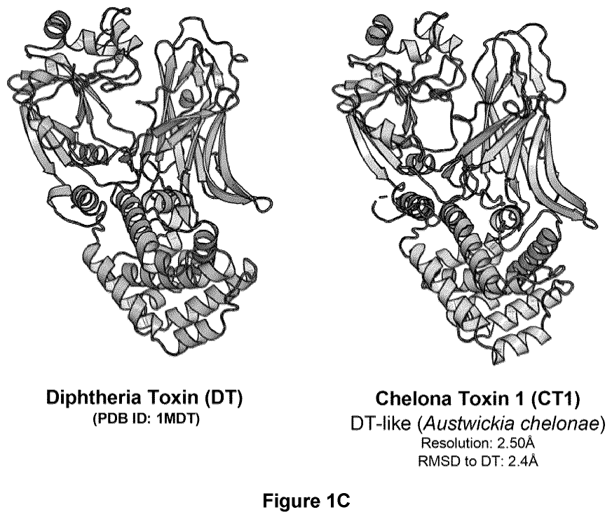

With the goal of finding an alternative DT-like immunotoxin platform unsusceptible to pre-existing anti-DT antibodies yet functionally active, the evolutionarily closest DT-like protein outside the Corynebacterium genus was chosen. By sequence, the DT-like protein from A. chelonae (CT1) is 38% identical to DT. The catalytically active residue (E148) and residues important for substrate (NAD) binding and coordination (H21, Y54, Y65) are all conserved, as is the furin recognition site, and the disulfide bond formed between C186 and C201 (in DT) is also present in the CT1 sequence (Table 1). Furthermore, key histidine residues and charged residues involved in the pH-dependent unfolding and pore formation of the translocation domain were found to be conserved in the predicted translocation domain of the sequence from CT1 (Table 1). To test whether the putative domains of the CT1 were functional, the protein was cloned, expressed and purified to study structurally and functionally (FIG. 1B).

Hanging drop vapour diffusion was used to obtain an x-ray crystallography structure of CT1. The protein successfully crystallized and diffracted to 2.50 Å. It was not possible to use the full-length DT structure (pdb 1MDT) as a search model for molecular replacement. However, using partial search models with 1MDT, the structure was solved (FIG. 1C). CT1 had a RMSD of 2.4 Å to DT, and retained the same three domain, Y-shaped architecture as DT.

### The C-Domain of CT1 is Functional and has the Same Intracellular Target as DT

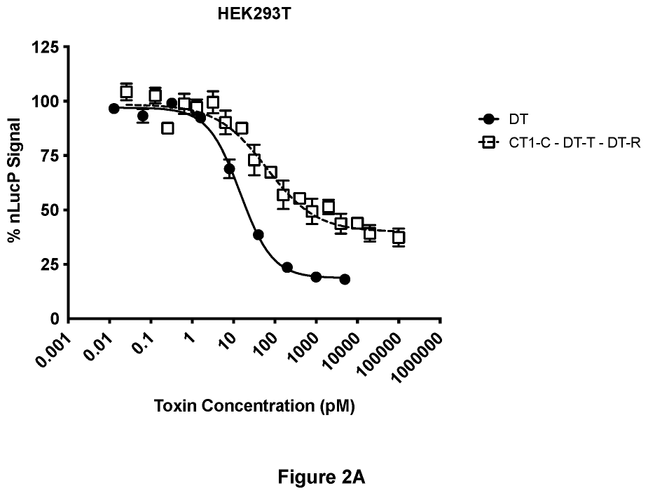

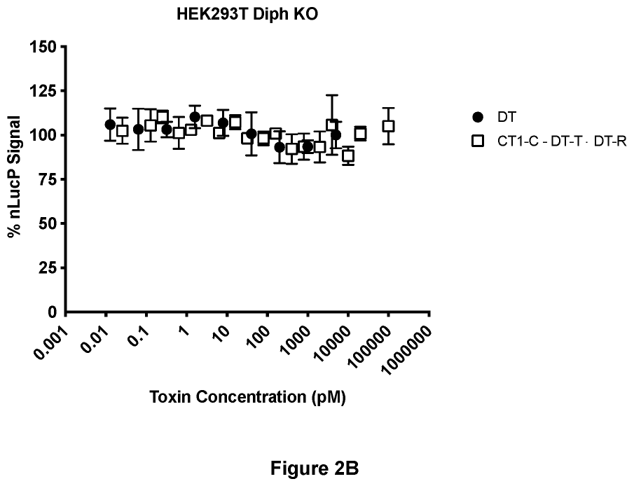

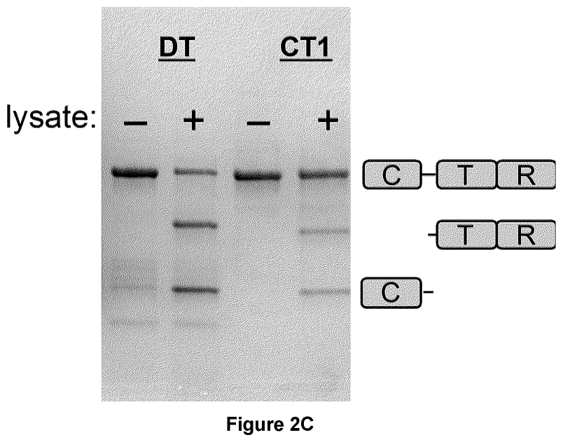

Structural alignments of CT1-C (the catalytic domain of CT1) to DT_{C} (the DT catalytic domain) show good structural conservation between key residues required for DT_{C} functionality. In order to test whether CT1-C was functional, a chimera was generated in which DT_{C} was replaced with CT1-C (referred to as CT1-C-DT_{T}-DT_{R}). The chimeric protein was tested on HEK293T cells with and without a gene knockout of DPH4 (DPH4^{-/-}). These cells are defective in the diphthamide synthesis pathway, and produce eukaryotic elongation factor 2 (eEF-2) without a diphthamide modification, and are therefore completely resistant to DT (FIGS. 2A and 2B). Wildtype HEK293T cells were susceptible to both wildtype DT and CT1-C-DT_{T}-DT_{R}, while the diphthamide knockout cells were not. This indicates that the catalytic domain of CT1 has the same intracellular target as DT.

### The C-Domain of CT1 is Efficiently Released

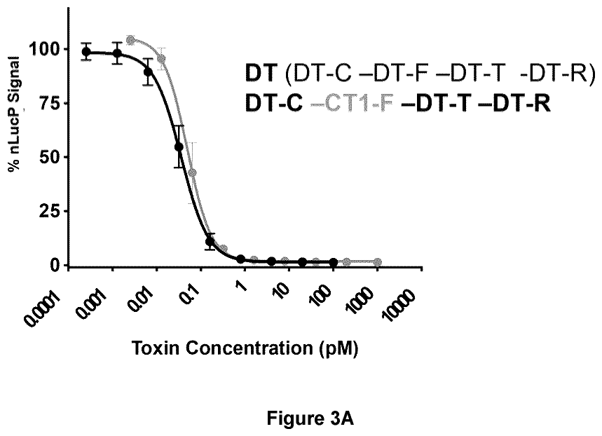

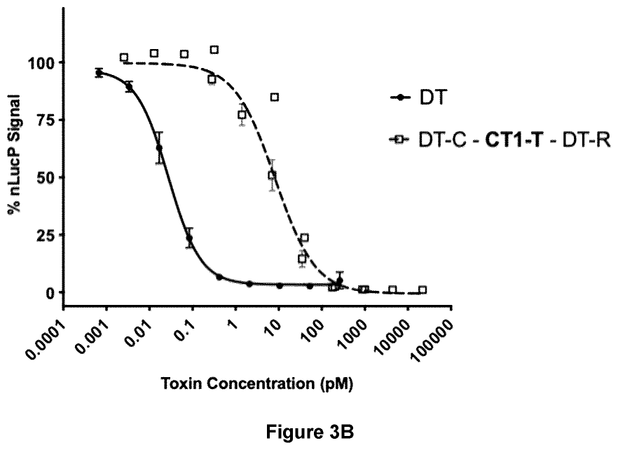

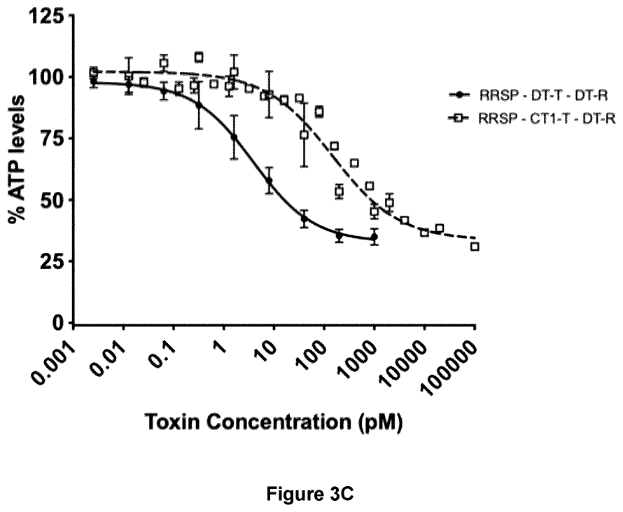

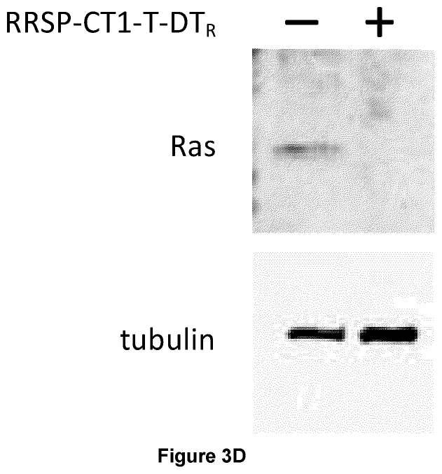

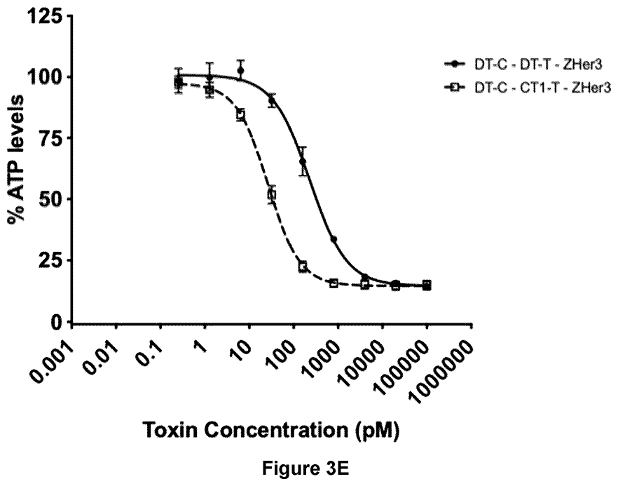

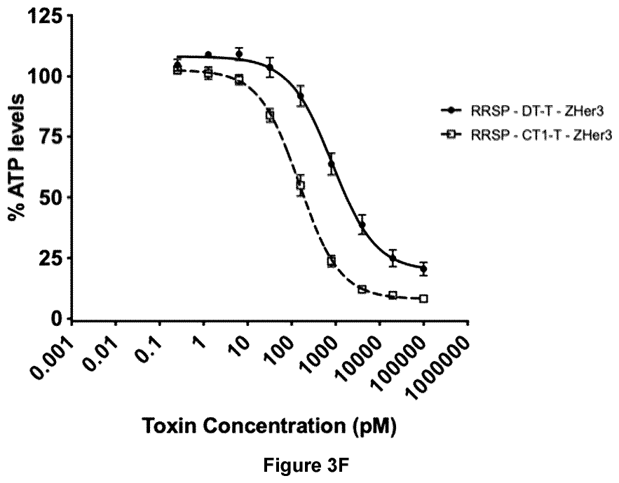

An important part of DT's intoxication mechanism is the release of the C-domain from the rest of the molecule, upon entry into the cytosol. The furin protease recognition site is conserved in CT1 (RAKR in CT1). To confirm that the furin site is recognized and the C-domain is released from the rest of the molecule, DT and CT1 were incubated with mammalian cell lysate overnight at 37 C. Both DT and CT1 were cleaved between the C and T domains (FIG. 2C). When DTC-CT1-F-DTT-DTR was tested on Vero cells, it was similarly toxic to DT (FIG. 3A), suggesting it is efficiently cleaved by furin.

### CT1 Contains a Functional Translocase

Endosomal acidification leads to the refolding and insertion of DT_{T} into the endosomal membrane, and subsequent translocation of the C-domain into the cytosol. This process is thought to be initiated and driven by nine charged residues in DT_{T}, of which six are conserved in CT1-T. To test whether CT1-T forms DT-like pores, the isolated T-domain was purified and tested in vitro for its capacity to release dye from liposomes (data not shown). CT1-T showed a pH-dependent increase in dye release, with the onset of dye release (interpreted as pore formation) at pH 5.5 (similar to DT_{T}).

It was further investigated whether the translocase deliver cargo into cells. To this end, a chimeric DT was generated in which DT_{T} was swapped for CT1-T (DT_{C}-CT1-T-DT_{R}) and measured its effect on protein synthesis. CT1-T successfully delivered DT_{C} into cells, as shown by the decrease in signal observed with increasing amounts of chimeric toxin (FIG. 3B. Furthermore, when DT_{C} was swapped for a non-native cargo (Ras/Rap1 Specific Protease; RRSP) targeting Ras oncoproteins, it was shown to successfully deliver this structurally distinct cargo (as shown by cell viability on CFPAC-1 cells, and western blot assessing intracellular Ras levels; FIGS. 3C and 3D). Taken together, these experiments demonstrated that CT1-T is a functional translocase capable of delivering various cargo into the cytosolic component of cells.

### The Translocation Domain of CT1 can Accommodate Diverse Receptor-Binding Domains

Having demonstrated that CT1-T could tolerate manipulation on the N-terminus (can translocate DT_{C} and RRSP), it was next assessed whether CT1-T could tolerate such manipulation on the C-terminus. To this end, a chimera was generated in which the receptor binding domains (DT_{R}) of DT_{C}-CT1-T-DT_{R} and RRSP-CT1-T-DT_{R} were swapped with a Her3 (human epidermal receptor 3) targeting affibody (ZHer3:08699, referred to hereafter as ZHer3) and tested these constructs on HPAF II cells (FIGS. 3E and 3F). It was found that not only could CT1-T be re-targeted to Her3 expressing cells, but surprisingly, that CT1-T-containing constructs appeared more efficient than DT_{T} at cargo delivery.

### CT1 is not Recognized or Neutralized by Human Serum

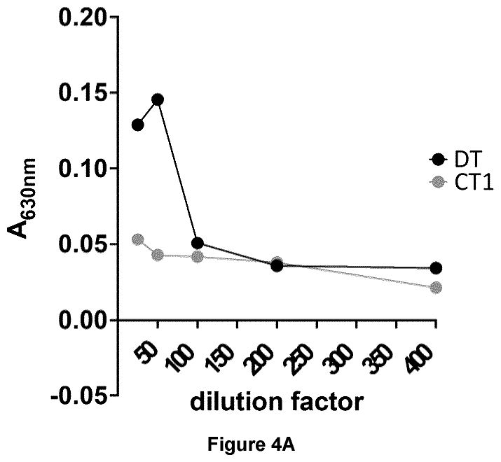

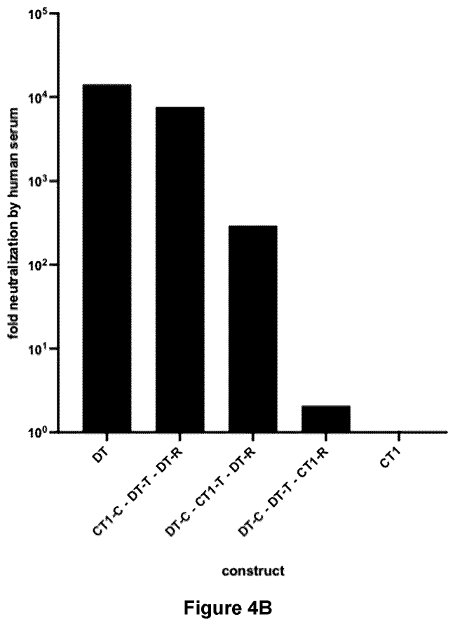

To quantify the level of pre-existing anti-DT or anti-CT1 antibodies in human serum, an ELISA assay was used in which either DT or CT1 was immobilized on high-bind plates and incubated with varying amounts of pooled human sera, after which an anti-IgG antibody conjugated to HRP was used to determine levels of antibody binding (FIG. 4A). While DT showed a dose-dependent increase in signal in the presence of human sera, CT1 did not, indicating that CT1 is not recognized by antibodies in human serum.

To confirm that DT is neutralized by antibodies in human sera, DT was incubated for 30 minutes with pooled human sera and then added it to Vero cells. A 6-log shift in toxicity of DT was observed, indicating neutralization by human sera (FIG. 4B). In contrast, CT1 was not neutralized by human sera. This indicates that CT1 is not neutralized by anti-DT antibodies. This provides CT1-based delivery vectors an advantage over DT-based vectors by circumventing the issue of pre-existing neutralizing anti-DT antibodies.

In addition to the translocation domain for CT1 (SEQ ID NO: 3), the translocation domains of SEQ ID NOs: 12 and 14 have also been established to be functional.

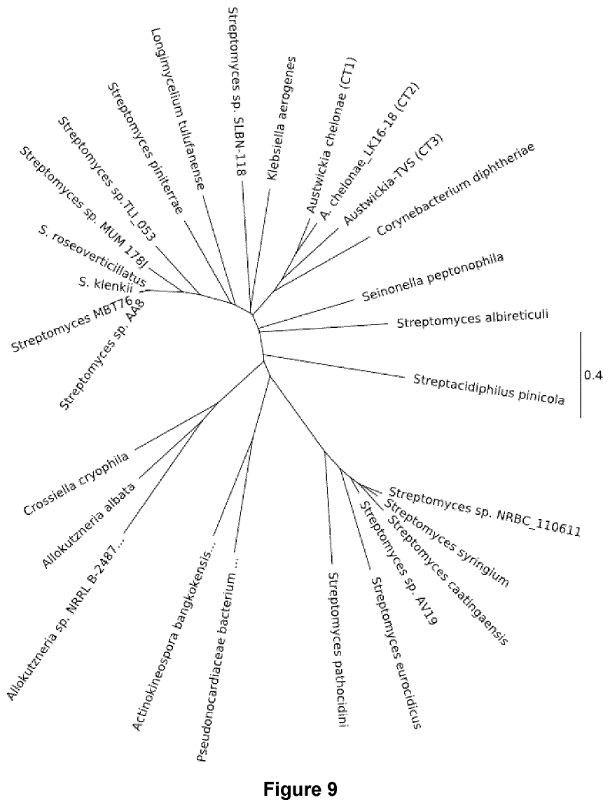

On the basis of the results obtained, the related polypeptide sequences from other bacterial strains and species described herein, and as set forth in SEQ ID NOs: 4 to 11, 13, 15 to 17, and 36 to 48 (and their related sequences), are also expected to be functional translocation domains that are active within polypeptide constructs as described herein. FIG. 9 provides a phylogenetic tree showing relationships between translocase domains.

### Example 2

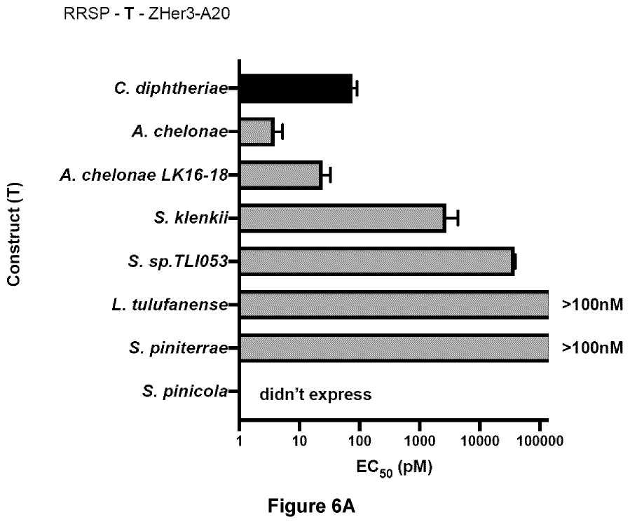

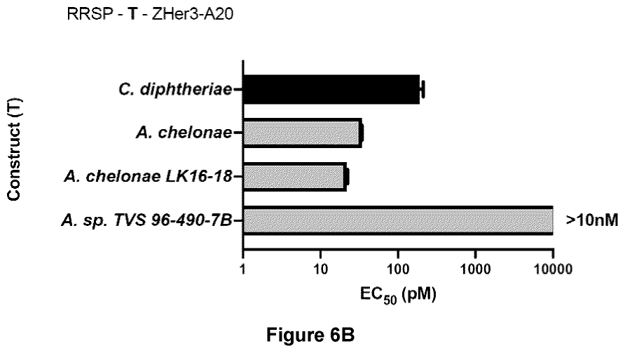

To evaluate the function of the novel translocases relative to DT, each translocase sequence, “T”, was cloned between the intracellular RAS cleaving enzyme RRSP (Ras/Rap1 Specific Peptidase, SEQ ID NO: 18; viz, the cargo) and a dual receptor binding domain known as ZHer3-A20 (consisting of an affibody against Her3, SEQ ID NO: 19, and a peptide against αvβ6 integrin known as A20FMDV2 (A20), SEQ ID NO: 20) yielding the construct RRSP-T-ZHer3-A20 (where “T” indicates the translocase). A range of protein concentrations of each identified construct was incubated with human pancreatic adenocarcinoma (HPAF-II) cells for 72-hours (FIG. 6A), and/or with epithelial-like cell (H358 cells) for 72-hours (FIG. 6B). As RRSP induces apoptosis after being delivered into cells, translocation was quantified by measuring the ability of each construct to kill human pancreatic adenocarcinoma cells after a 72-hour incubation.

FIGS. 6A and 6B show results of functional characterization of translocases. Using the RRSP-T-ZHer3-A20 template, the function of each translocase was quantified. The concentration of a given construct that resulted in reduction of cell viability by 50% of maximal toxicity (EC50) is represented in the bar graph. A lower value represents a more efficient translocase. The translocase from S. pinicola was not expressed and so could not be evaluated. The translocases from S. piniterrae and L. tulufanense were determined to be non-functional in the assay as no toxicity was observed up to the highest dose tested (100 nM). The remaining five translocases were functional in the assay. The translocases of the proteins from A. chelonae (SEQ ID NO: 2) and A. chelonae LK16-18 (SEQ ID NO: 17) were the most active on cells and better than DT's translocase. Activity levels observed indicate that the RRSP is efficiently released.

The translocase domains Streptomyces albireticuli and Seinonella peptonophila have also been shown to be functional (see Sugiman-Marangos et al. 2022, which is incorporated by reference in its entirety).

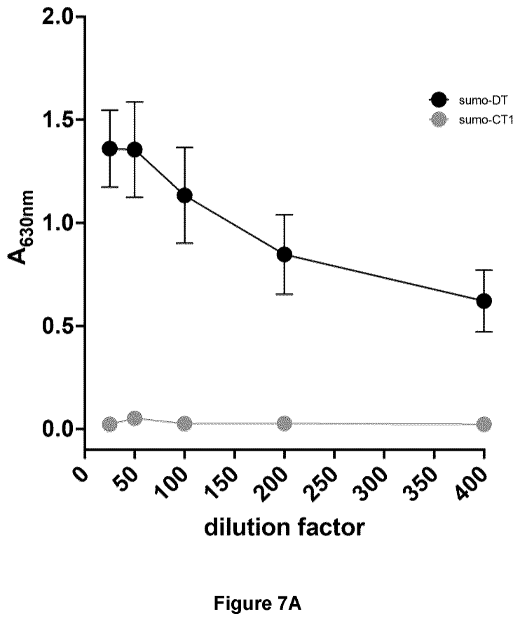

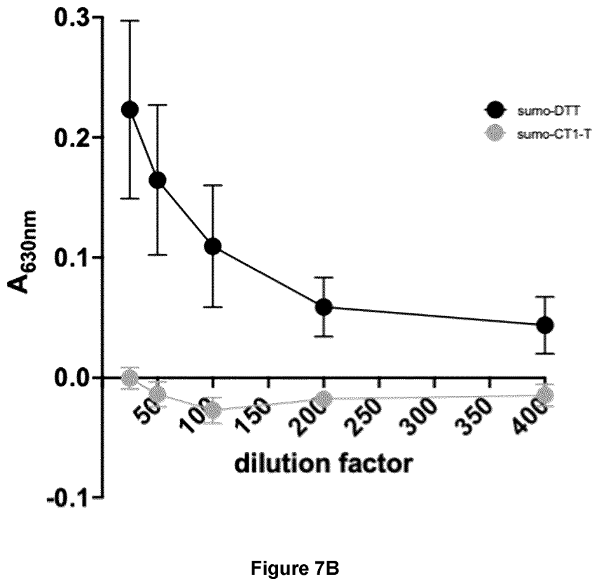

The diphtheria toxoid vaccine is part of global vaccination programs that serve to protect against the disease diphtheria. Anti-DT antibodies in human sera prevent the actions of DT by binding to DT and neutralizing its function. Unfortunately, these same antibodies also bind to and neutralize DT-based therapeutics. An ELISA was performed to evaluate the degree to which anti-DT antibodies in human sera recognize full-length toxins DT and CT1 (SEQ ID NO: 2), and the translocases DT-T and CT1-T (SEQ ID NO: 3). As shown in the ELISA data in FIG. 7A, antibodies in human sera recognize DT, but show no binding to CT1. Similarly, high titres are seen against the Translocase from DT, but not CT1 (FIG. 7B).

FIGS. 7A and 7B together show that pre-existing anti-DT antibodies in human serum do not bind or neutralize CT1-based immunotoxins. The respective protein was immobilized on plates and incubated with varying amounts of pediatric human serum sample, after which an anti-IgG antibody conjugated to HRP was used to determine levels of antibody binding. DT and DT-T both showed a dose dependent increase in signal with human serum, while CT1 and CT1-T did not. This indicates that full length CT1 and just its translocase do not contain epitopes recognized by antibodies in human serum.

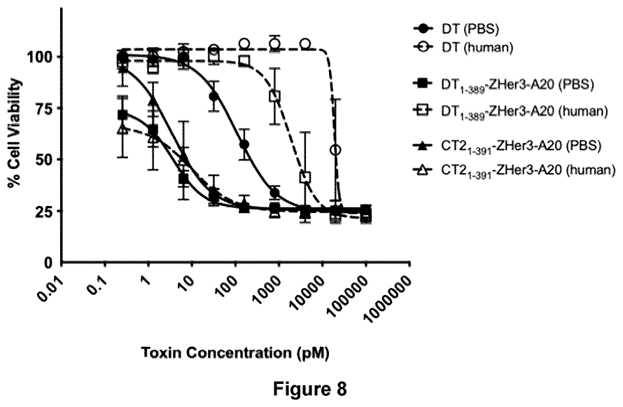

Next, to evaluate the degree to which pre-existing anti-DT antibodies in human sera neutralize the function of DT-based therapeutics as well as the corresponding therapeutics based on novel translocation domains, DT- and the CT-based immunotoxins from A. chelonae LK16-18 were incubated with human sera. DT- and CT-based immunotoxins were cloned and purified, where the C- and T-domains of the respective toxin were recombinantly attached to ZHer3-A20 (either DT₁₋₃₈₉-ZHer3-A20 or CT2₁₋₃₉₁-ZHer3-A20). As shown in FIG. 8, in the absence of human sera (PBS), DT, DT₁₋₃₈₉-ZHer-3-A20 and CT2₁₋₃₉₁-ZHer3-A20 were all toxic to human cells. In the presence of human sera (human), a rightward shift in the toxicity curve is seen for DT and DT₁₋₃₈₉-ZHer3-A20, indicating the human sera inhibited/neutralized their function. Conversely, human sera had no effect on the toxicity curve for CT2₁₋₃₉₁-ZHer3-A20, indicating that human sera did not recognize or inhibit/neutralize CT-based therapeutics.

FIG. 8 shows that anti-DT antibodies do not neutralize CT-based immunotoxins. Immunotoxins were incubated for 30 minutes with either a PBS control or human serum, and then added to cells. Cell viability was assessed at 72 hours, and it was found that while DT₁₋₃₈₉-ZHer3-A20 had ~3-log decrease in toxicity upon human serum incubation, CT2₁₋₃₉₁-ZHer3-A20 had no shift.

### Example 3

### Example Annotated Construct Sequences

RRSP-T (C. diphtheriae)-ZHer3-A20
(SEQ ID NO: 22)
GDKTKVVVDLAQIFTVQELKERAKVFAKPIGASYQGILDQLDLVHQAKGRDQI
AASFELNKKINDYIAEHPTSGRNQALTQLKEQVTSALFIGKMQVAQAGIDAIAQTRPELAARIF
MVAIEEANGKHVGLTDMMVRWANEDPYLAPKHGYKGETPSDLGFDAKYHVDLGEHYADFK
QWLETSQSNGLLSKATLDESTKTVHLGYSYQELQDLTGAESVQMAFYFLKEAAKKADPISG
DSAEMILLKKFADQSYLSQLDSDRMDQIEGIYRSSHETDIDAWDRRYSGTGYDELTNKLASA
TGVDEQLAVLLDDRKGLLIGEVHGSDVNGLRFVNEQMDALKKQGVTVIGLEHLRSDLAQPLI
DRYLATGVMSSELSAMLKTKHLDVTLFENARANGMRIVALDANSSARPNVQGTEHGLMYRA
GAANNIAVEVLQNLPDGEKFVAIYGKAHLQSHKGIEGFVPGITHRLDLPALKVSDSNQFTVE
QDDVSLRVVYDDVANKPKITFKGSLGGGGSGGGGSCAGNRVRRSVGSSLSCINLDWDVIR
DKTKTKIESLKEHGPIKNKMSESPNKTVSEEKAKQYLEEFHQTALEHPELSELKTVTGTNPVF
AGANYAAWAVNVAQVIDSETADNLEKTTAALSILPGIGSVMGIADGAVHHNTEEIVAQSIALS
SLMVAQAIPLVGELVDIGFAAYNFVESIINLFQVVHNSYNRPAYSPGHKTQPFGGGGSGGG
GSAEAKYAKEKYNAYYEIWQLPNLTKYQKAAFIGKLQDDPSQSSELLSEAKKLNDSQAPKG
GGGSGGGGSNAVPNLRGDLQVLAQKVARTRQALVPRGSAWSHPQFEK

In the above:
residues 1-510=RRSP
residues 511-724=T domain from C. diphtheriae
residues 725-734=G4S2 linker
residues 735-792=ZHer3:08699 affibody
residues 793-802=G4S2 linker
residues 803-822=A20FMDV2 peptide
residues 823-840=thrombin cleavage site and strep-tag II

RRSP-T (A. chelonae)-ZHer3-A20
(SEQ ID NO: 23)
GDKTKVVVDLAQIFTVQELKERAKVFAKPIGASYQGILDQLDLVHQAKGRDQI
AASFELNKKINDYIAEHPTSGRNQALTQLKEQVTSALFIGKMQVAQAGIDAIAQTRPELAARIF
MVAIEEANGKHVGLTDMMVRWANEDPYLAPKHGYKGETPSDLGFDAKYHVDLGEHYADFK
QWLETSQSNGLLSKATLDESTKTVHLGYSYQELQDLTGAESVQMAFYFLKEAAKKADPISG
DSAEMILLKKFADQSYLSQLDSDRMDQIEGIYRSSHETDIDAWDRRYSGTGYDELTNKLASA
TGVDEQLAVLLDDRKGLLIGEVHGSDVNGLRFVNEQMDALKKQGVTVIGLEHLRSDLAQPLI
DRYLATGVMSSELSAMLKTKHLDVTLFENARANGMRIVALDANSSARPNVQGTEHGLMYRA
GAANNIAVEVLQNLPDGEKFVAIYGKAHLQSHKGIEGFVPGITHRLDLPALKVSDSNQFTVE
QDDVSLRVVYDDVANKPKITFKGSLGGGGSGGGGSCAGNRVRRSVGSSLSCLSKINWKNV
REKADALTKKVHADKEFMDKLSTHHQRGEAPSVEKTTALHNALLEHESFSALKGARASGKV
GAAASTAAWGVAVAQAFTDPKADALTKTAATLSVVPGLGQALGIADGIKHENTEEIVVQSISL
AGLLAAQAIPVVGEAVDFGLLVYQLVETIVDLATHLSSAAANPAYSPGHKTQPFGGGGSGG
GGSAEAKYAKEKYNAYYEIWQLPNLTKYQKAAFIGKLQDDPSQSSELLSEAKKLNDSQAPK
GGGGSGGGGSNAVPNLRGDLQVLAQKVARTRQALVPRGSAWSHPQFEK

In the above:
residues 1-510=RRSP
residues 511-520=G4S2 linker
residues 521-536=C. diphtheriae sequence with furin protease recognition site
residues 537-711=T domain from A. chelonae
residues 712-723=linker sequence from C. diphtheriae
residues 724-733=G4S2 linker
residues 734-791=ZHer3:08699 affibody
residues 792-801=G4S2 linker
residues 802-821=A20FMDV2 peptide
residues 822-839=thrombin cleavage site and strep-tag II

RRSP-T (A. chelonae LK16-18)-ZHer3-A20
(SEQ ID NO: 24)
GDKTKVVVDLAQIFTVQELKERAKVFAKPIGASYQGILDQLDLVHQAKGRDQI
AASFELNKKINDYIAEHPTSGRNQALTQLKEQVTSALFIGKMQVAQAGIDAIAQTRPELAARIF
MVAIEEANGKHVGLTDMMVRWANEDPYLAPKHGYKGETPSDLGFDAKYHVDLGEHYADFK
QWLETSQSNGLLSKATLDESTKTVHLGYSYQELQDLTGAESVQMAFYFLKEAAKKADPISG
DSAEMILLKKFADQSYLSQLDSDRMDQIEGIYRSSHETDIDAWDRRYSGTGYDELTNKLASA
TGVDEQLAVLLDDRKGLLIGEVHGSDVNGLRFVNEQMDALKKQGVTVIGLEHLRSDLAQPLI
DRYLATGVMSSELSAMLKTKHLDVTLFENARANGMRIVALDANSSARPNVQGTEHGLMYRA
GAANNIAVEVLQNLPDGEKFVAIYGKAHLQSHKGIEGFVPGITHRLDLPALKVSDSNQFTVE
QDDVSLRVVYDDVANKPKITFKGSLGGGGSGGGGSCAGNRVRRSVGSSLSCLSKVKWEQ
VREKSKKIIDNVKDNPEFMKKLSAHHERGSAPTTEKITALHNELLDHESFSALKGARSSAGTA
ATAASAAAWGLAVAQAFTNPKADDLTKATAVLSAVPGLGQALGIADGIKHHNTEEIVVQSISL
TALIAAQAIPVVGELVDFGLLAYQLVESIIDLTRQLSVITANPAYSPGHKTQPFGGGGSGGGG
SAEAKYAKEKYNAYYEIWQLPNLTKYQKAAFIGKLQDDPSQSSELLSEAKKLNDSQAPKGG
GGSGGGGSNAVPNLRGDLQVLAQKVARTRQALVPRGSAWSHPQFEK

In the above:
residues 1-510=RRSP
residues 511-520=G4S2 linker
residues 521-536=C. diphtheriae sequence with furin protease recognition site
residues 537-712=T domain from A. chelonae LK16-18
residues 713-724=linker sequence from C. diphtheriae
residues 725-734=G4S2 linker
residues 735-792=ZHer3:08699 affibody
residues 793-802=G4S2 linker
residues 803-822=A20FMDV2 peptide
residues 823-840=thrombin cleavage site and strep-tag II

RRSP-T (A. TVS 96-490-7B)-ZHer3-A20
(SEQ ID NO: 25)
GDKTKVVVDLAQIFTVQELKERAKVFAKPIGASYQGILDQLDLVHQAKGRDQI
AASFELNKKINDYIAEHPTSGRNQALTQLKEQVTSALFIGKMQVAQAGIDAIAQTRPELAARIF
MVAIEEANGKHVGLTDMMVRWANEDPYLAPKHGYKGETPSDLGFDAKYHVDLGEHYADFK
QWLETSQSNGLLSKATLDESTKTVHLGYSYQELQDLTGAESVQMAFYFLKEAAKKADPISG
DSAEMILLKKFADQSYLSQLDSDRMDQIEGIYRSSHETDIDAWDRRYSGTGYDELTNKLASA
TGVDEQLAVLLDDRKGLLIGEVHGSDVNGLRFVNEQMDALKKQGVTVIGLEHLRSDLAQPLI
DRYLATGVMSSELSAMLKTKHLDVTLFENARANGMRIVALDANSSARPNVQGTEHGLMYRA
GAANNIAVEVLQNLPDGEKFVAIYGKAHLQSHKGIEGFVPGITHRLDLPALKVSDSNQFTVE
QDDVSLRVVYDDVANKPKITFKGSLGGGGSGGGGSCAGNRVRRSVGSSLSCLSNIKWERV
RQKSEELIKKLKDDENVKKAVQERKEGVKPTSDDLQNLHKALIDHESFKELKSVHSNGVKA
MDAANAALWGANVARVFSDSKSDGLEKATAALAAVPGLGQVMGVADGVTHHNTEEVVVQ
SVALAGFIAAQAIPVVGEIVDIGVLAYQFVEGVIDLSRQMMTSNARPAYSPGHKTQPFGGGG
SGGGGSAEAKYAKEKYNAYYEIWQLPNLTKYQKAAFIGKLQDDPSQSSELLSEAKKLNDSQ
APKGGGGSGGGGSNAVPNLRGDLQVLAQKVARTRQALVPRGSAWSHPQFEK

In the above:
residues 1-510=RRSP
residues 511-520=G4S2 linker
residues 521-536=C. diphtheriae sequence with furin protease recognition site
residues 537-711=T domain from A. TVS 96-490-7B
residues 712-723=linker sequence from C. diphtheriae
residues 724-733=G4S2 linker
residues 734-791=ZHer3:08699 affibody
residues 792-801=G4S2 linker
residues 802-821=A20FMDV2 peptide
residues 822-839=thrombin cleavage site and strep-tag II

RRSP-T (S. klenkii)-ZHer3-A20
(SEQ ID NO: 26)
GDKTKVVVDLAQIFTVQELKERAKVFAKPIGASYQGILDQLDLVHQAKGRDQI
AASFELNKKINDYIAEHPTSGRNQALTQLKEQVTSALFIGKMQVAQAGIDAIAQTRPELAARIF
MVAIEEANGKHVGLTDMMVRWANEDPYLAPKHGYKGETPSDLGFDAKYHVDLGEHYADFK
QWLETSQSNGLLSKATLDESTKTVHLGYSYQELQDLTGAESVQMAFYFLKEAAKKADPISG
DSAEMILLKKFADQSYLSQLDSDRMDQIEGIYRSSHETDIDAWDRRYSGTGYDELTNKLASA
TGVDEQLAVLLDDRKGLLIGEVHGSDVNGLRFVNEQMDALKKQGVTVIGLEHLRSDLAQPLI
DRYLATGVMSSELSAMLKTKHLDVTLFENARANGMRIVALDANSSARPNVQGTEHGLMYRA
GAANNIAVEVLQNLPDGEKFVAIYGKAHLQSHKGIEGFVPGITHRLDLPALKVSDSNQFTVE
QDDVSLRVVYDDVANKPKITFKGSLGGGGSGGGGSCAGNRVRRSVGSSLSCLPVDWDKV
EEKAKATAKKVAQDAEHVEKLPKRSPKGPTWGEAHSTAELSHAKVREVSGAHVAASAAGV
GTWVYGMAKTFSDKDATTLDKVAVTGAVVPGLGQALGIADGIQHGDPEAIAVNAVALAALA
AAQVVPVVGEVVDAVLLTEQLVEVLVDVFRTATADPAYSPGHKTQPFGGGGSGGGGSAEA
KYAKEKYNAYYEIWQLPNLTKYQKAAFIGKLQDDPSQSSELLSEAKKLNDSQAPKGGGGSG
GGGSNAVPNLRGDLQVLAQKVARTRQALVPRGSAWSHPQFEK

In the above:
residues 1-510=RRSP
residues 511-520=G4S2 linker
residues 521-536=C. diphtheriae sequence with furin protease recognition site
residues 537-700=T domain from S. klenkii
residues 701-712=linker sequence from C. diphtheriae
residues 713-722=G4S2 linker
residues 723-780=ZHer3:08699 affibody
residues 781-790=G4S2 linker
residues 791-810=A20FMDV2 peptide
residues 811-828=thrombin cleavage site and strep-tag II

RRSP-T (S. sp TLI053)-ZHer3-A20
(SEQ ID NO: 27)
GDKTKVVVDLAQIFTVQELKERAKVFAKPIGASYQGILDQLDLVHQAKGRDQI
AASFELNKKINDYIAEHPTSGRNQALTQLKEQVTSALFIGKMQVAQAGIDAIAQTRPELAARIF
MVAIEEANGKHVGLTDMMVRWANEDPYLAPKHGYKGETPSDLGFDAKYHVDLGEHYADFK
QWLETSQSNGLLSKATLDESTKTVHLGYSYQELQDLTGAESVQMAFYFLKEAAKKADPISG
DSAEMILLKKFADQSYLSQLDSDRMDQIEGIYRSSHETDIDAWDRRYSGTGYDELTNKLASA
TGVDEQLAVLLDDRKGLLIGEVHGSDVNGLRFVNEQMDALKKQGVTVIGLEHLRSDLAQPLI
DRYLATGVMSSELSAMLKTKHLDVTLFENARANGMRIVALDANSSARPNVQGTEHGLMYRA
GAANNIAVEVLQNLPDGEKFVAIYGKAHLQSHKGIEGFVPGITHRLDLPALKVSDSNQFTVE
QDDVSLRVVYDDVANKPKITFKGSLGGGGSGGGGSCAGNRVRRSVGSSLSCIPAASWEAI
EHRSKEMASAVARDTEYTKSLPNRHPKGPTWSEAHTTSTSTHARVSAKSGAHVAVGAFAV
GSWIYGMSETFANKNVTTLDKAAATVAIVPGIGHALGIAAALEHHDIEGVVVNAISIAALAAAQ
VVPVVGEIVDAALLAEQLVEVLVHVFRASTTEPAYSPGHKTQPFGGGGSGGGGSAEAKYAK
EKYNAYYEIWQLPNLTKYQKAAFIGKLQDDPSQSSELLSEAKKLNDSQAPKGGGGSGGGG
SNAVPNLRGDLQVLAQKVARTRQALVPRGSAWSHPQFEK

In the above:
residues 1-510=RRSP
residues 511-520=G4S2 linker
residues 521-536=C. diphtheriae sequence with furin protease recognition site
residues 537-701=T domain from S. sp TLI053
residues 702-713=linker sequence from C. diphtheriae
residues 714-723=G4S2 linker
residues 724-781=ZHer3:08699 affibody
residues 782-791=G4S2 linker
residues 792-811=A20FMDV2 peptide
residues 812-829=thrombin cleavage site and strep-tag II

RRSP-T (L. tulufanense)-ZHer3-A20
(SEQ ID NO: 28)
GDKTKVVVDLAQIFTVQELKERAKVFAKPIGASYQGILDQLDLVHQAKGRDQI
AASFELNKKINDYIAEHPTSGRNQALTQLKEQVTSALFIGKMQVAQAGIDAIAQTRPELAARIF
MVAIEEANGKHVGLTDMMVRWANEDPYLAPKHGYKGETPSDLGFDAKYHVDLGEHYADFK
QWLETSQSNGLLSKATLDESTKTVHLGYSYQELQDLTGAESVQMAFYFLKEAAKKADPISG
DSAEMILLKKFADQSYLSQLDSDRMDQIEGIYRSSHETDIDAWDRRYSGTGYDELTNKLASA
TGVDEQLAVLLDDRKGLLIGEVHGSDVNGLRFVNEQMDALKKQGVTVIGLEHLRSDLAQPLI
DRYLATGVMSSELSAMLKTKHLDVTLFENARANGMRIVALDANSSARPNVQGTEHGLMYRA
GAANNIAVEVLQNLPDGEKFVAIYGKAHLQSHKGIEGFVPGITHRLDLPALKVSDSNQFTVE
QDDVSLRVVYDDVANKPKITFKGSLGGGGSGGGGSCAGNRVRRSVGSSLSCERKTLDKLD
RKKIETRAKQVISKLAQDGDLRRELPRRTTTGHAHEEVRGVLAKSRGALHEIHGAATTVAMP
LATVAWVEDMARVFREKNATTLDKAATVSEIVPVAGQVLGMADGIAHRDAETVAVNAVVLA
AIAVSQAVPVVGELVDLGLTAYAVVDVVVRLFGPAREVPITQESYWPAYSPGHKTQPFGGG
GSGGGGSAEAKYAKEKYNAYYEIWQLPNLTKYQKAAFIGKLQDDPSQSSELLSEAKKLNDS
QAPKGGGGSGGGGSNAVPNLRGDLQVLAQKVARTRQALVPRGSAWSHPQFEK

In the above:
residues 1-510=RRSP
residues 511-520=G4S2 linker
residues 521-536=C. diphtheriae sequence with furin protease recognition site
residues 537-714=T domain from L. tulufanense
residues 715-726=linker sequence from C. diphtheriae
residues 727-736=G4S2 linker
residues 737-794=ZHer3:08699 affibody
residues 795-804=G4S2 linker
residues 805-824=A20FMDV2 peptide
residues 825-842=thrombin cleavage site and strep-tag II

RRSP-T (S. piniterrae)-ZHer3-A20
(SEQ ID NO: 29)
GDKTKVVVDLAQIFTVQELKERAKVFAKPIGASYQGILDQLDLVHQAKGRDQI
AASFELNKKINDYIAEHPTSGRNQALTQLKEQVTSALFIGKMQVAQAGIDAIAQTRPELAARIF
MVAIEEANGKHVGLTDMMVRWANEDPYLAPKHGYKGETPSDLGFDAKYHVDLGEHYADFK
QWLETSQSNGLLSKATLDESTKTVHLGYSYQELQDLTGAESVQMAFYFLKEAAKKADPISG
DSAEMILLKKFADQSYLSQLDSDRMDQIEGIYRSSHETDIDAWDRRYSGTGYDELTNKLASA
TGVDEQLAVLLDDRKGLLIGEVHGSDVNGLRFVNEQMDALKKQGVTVIGLEHLRSDLAQPLI
DRYLATGVMSSELSAMLKTKHLDVTLFENARANGMRIVALDANSSARPNVQGTEHGLMYRA
GAANNIAVEVLQNLPDGEKFVAIYGKAHLQSHKGIEGFVPGITHRLDLPALKVSDSNQFTVE
QDDVSLRVVYDDVANKPKITFKGSLGGGGSGGGGSCAGNRVRRSVGSSLSCELAKDKAR
DILSEAGNEVSLPQRDSDGLSKQEITATAEATRSKLGTGVHGAVSAAMVADWAHDVARTFA
DPKATKLDKAAAVTAIAPVIGQAVNIADGIQHHDKKTIVVNSLVLAAVVAAQAVPAVGEVVDA
AIVADFVVEKLVGWFTPTAKPGPEHVAPAYSPGHKTQPFGGGGSGGGGSAEAKYAKEKYN
AYYEIWQLPNLTKYQKAAFIGKLQDDPSQSSELLSEAKKLNDSQAPKGGGGSGGGGSNAV
PNLRGDLQVLAQKVARTRQALVPRGSAWSHPQFEK

In the above:
residues 1-510=RRSP
residues 511-520=G4S2 linker
residues 521-536=C. diphtheriae sequence with furin protease recognition site
residues 537-695=T domain from S. piniterrae
residues 696-707=linker sequence from C. diphtheriae
residues 708-717=G4S2 linker
residues 718-775=ZHer3:08699 affibody
residues 776-786=G4S2 linker
residues 786-805=A20FMDV2 peptide
residues 806-823=thrombin cleavage site and strep-tag II

RRSP-T (S. pinicola)-ZHer3-A20
(SEQ ID NO: 30)
GDKTKVVVDLAQIFTVQELKERAKVFAKPIGASYQGILDQLDLVHQAKGRDQI
AASFELNKKINDYIAEHPTSGRNQALTQLKEQVTSALFIGKMQVAQAGIDAIAQTRPELAARIF
MVAIEEANGKHVGLTDMMVRWANEDPYLAPKHGYKGETPSDLGFDAKYHVDLGEHYADFK
QWLETSQSNGLLSKATLDESTKTVHLGYSYQELQDLTGAESVQMAFYFLKEAAKKADPISG
DSAEMILLKKFADQSYLSQLDSDRMDQIEGIYRSSHETDIDAWDRRYSGTGYDELTNKLASA
TGVDEQLAVLLDDRKGLLIGEVHGSDVNGLRFVNEQMDALKKQGVTVIGLEHLRSDLAQPLI
DRYLATGVMSSELSAMLKTKHLDVTLFENARANGMRIVALDANSSARPNVQGTEHGLMYRA
GAANNIAVEVLQNLPDGEKFVAIYGKAHLQSHKGIEGFVPGITHRLDLPALKVSDSNQFTVE
QDDVSLRVVYDDVANKPKITFKGSLGGGGSGGGGSCAGNRVRRSVGSSLSCKVNWKDAY
DRSMNIAQDIDGSAEFRATAPARPAAGKAMPEADVTRLVSTSEDFLEKSTKTSGVKKALKAI
NNQKMISWGSLLSNALANSHTWSDKNATNLDKAYAVVGGVPVLGEVIGIASGIDKQDAESIA
VNTLSLVGIVAATVCPPLGATVEFVMIGYTAIKLMLSWFTVETIPAYSPGHKTQPFGGGGSG
GGGSAEAKYAKEKYNAYYEIWQLPNLTKYQKAAFIGKLQDDPSQSSELLSEAKKLNDSQAP
KGGGGSGGGGSNAVPNLRGDLQVLAQKVARTRQALVPRGSAWSHPQFEK

In the above:
residues 1-510=RRSP
residues 511-520=G4S2 linker
residues 521-536=C. diphtheriae sequence with furin protease recognition site
residues 537-712=T domain from S. pinicola
residues 713-724=linker sequence from C. diphtheriae
residues 725-734=G4S2 linker
residues 735-792=ZHer3:08699 affibody
residues 793-802=G4S2 linker
residues 803-822=A20FMDV2 peptide
residues 823-840=thrombin cleavage site and strep-tag II

DT-Her3-A20
(SEQ ID NO: 31)
GADDVVDSSKSFVMENFSSYHGTKPGYVDSIQKGIQKPKSGTQGNYDDDW
KGFYSTDNKYDAAGYSVDNENPLSGKAGGVVKVTYPGLTKVLALKVDNAETIKKELGLSLTE
PLMEQVGTEEFIKRFGDGASRVVLSLPFAEGSSSVEYINNWEQAKALSVELEINFETRGKRG
QDAMYEYMAQACAGNRVRRSVGSSLSCINLDWDVIRDKTKTKIESLKEHGPIKNKMSESPN
KTVSEEKAKQYLEEFHQTALEHPELSELKTVTGTNPVFAGANYAAWAVNVAQVIDSETADN
LEKTTAALSILPGIGSVMGIADGAVHHNTEEIVAQSIALSSLMVAQAIPLVGELVDIGFAAYNFV
ESIINLFQVVHNSYNRPAYSPGHKTQPFGGGGSGGGGSAEAKYAKEKYNAYYEIWQLPNLT
KYQKAAFIGKLQDDPSQSSELLSEAKKLNDSQAPKGGGGSGGGGSNAVPNLRGDLQVLAQ
KVARTRQALVPRGSAWSHPQFEK

In the above:
residues 1-389=sequence from C. diphtheriae
residues 390-399=G4S2 linker
residues 400-457=Her3:08699 affibody
residues 458-467=G4S2 linker
residues 468-487=A20FMDV2 peptide
residues 487-505=thrombin cleavage site and strep-tag II

CT2-ZHer3-A20
(SEQ ID NO: 32)
YANDAVIADQSKTVDSFTSYHGAKPESFESVLAGIKKPESGSQGNHDPEWK
GFYTTDNKHAAAGYTVSDESVMTGKAGGVVKVTYPGKTRVLAVKPLSATELKTYLGLAADK
PLIDQLNNKDFINKFGEGASRVVLQMPFADGTSDVEYIHNWEDATQLQVATEVRFDNLGKR
GQDEMNRYMNLANCPSVSAVRVKRNPAKLCLSKVKWEQVREKSKKIIDNVKDNPEFMKKL
SAHHERGSAPTTEKITALHNELLDHESFSALKGARSSAGTAATAASAAAWGLAVAQAFTNP
KADDLTKATAVLSAVPGLGQALGIADGIKHHNTEEIVVQSISLTALIAAQAIPVVGELVDFGLLA
YQLVESIIDLTRQLSVITANPPTEVTHSSKLAGGGGSGGGGSAEAKYAKEKYNAYYEIWQLP
NLTKYQKAAFIGKLQDDPSQSSELLSEAKKLNDSQAPKGGGGSGGGGSNAVPNLRGDLQV
LAQKVARTRQALVPRGSAWSHPQFEK

In the above:
residues 1-391=sequence from A. chelonae LK16-18
residues 392-401=G4S2 linker
residues 402-459=Her3:08699 affibody
residues 460-469=G4S2 linker
residues 470-489=A20FMDV2 peptide
residues 491-507=thrombin cleavage site and strep-tag II.

TABLE 1
Table of Sequences
Full-length Proteins

| SEQ | | | GenBank |
|---|---|---|---|
| ID NO: | Species | Sequence | Accession |
| 1 | Corynebacterium | GADDVVDSSKSFVMENFSSYHGTKPGYVDSIQKGIQKPKS | From |
| | diphtheriae | GTQGNYDDDWKGFYSTDNKYDAAGYSVDNENPLSGKAGGV | WP_072564851.1 |
| | | VKVTYPGLTKVLALKVDNAETIKKELGLSLTEPLMEQVGT | |
| | | EEFIKRFGDGASRVVLSLPFAEGSSSVEYINNWEQAKALS | |
| | | VELEINFETRGKRGQDAMYEYMAQACAGNRVRRSVGSSLS | |
| | | CINLDWDVIRDKTKTKIESLKEHGPIKNKMSESPNKTVSE | |
| | | EKAKQYLEEFHQTALEHPELSELKTVTGTNPVFAGANYAA | |
| | | WAVNVAQVIDSETADNLEKTTAALSILPGIGSVMGIADGA | |
| | | VHHNTEEIVAQSIALSSLMVAQAIPLVGELVDIGFAAYNF | |
| | | VESIINLFQVVHNSYNRPAYSPGHKTQPFLHDGYAVSWNT | |
| | | VEDSIIRTGFQGESGHDIKITAENTPLPIAGVLLPTIPGK | |
| | | LDVNKSKTHISVNGRKIRMRCRAIDGDVTFCRPKSPVYVG | |
| | | NGVHANLHVAFHRSSSEKIHSNEISSDSIGVLGYQKTVDH | |
| | | TKVNSKLSLFFEIKS | |
| 2 | Austwickia chelonae | YANDVVLKDQSTTVDSFTSYHGAKPESFNAVLTGIKKPEK | N/A |
| | (CT1) | GSQGNNDPDWKGFYTTDNKHAAAGYTVSDESVLSGKAGGV | |
| | | VRVTYPGKTRILAVKSLSAAELKGKLGLDSAKPLIDQLND | |
| | | KSFLEKYGDGANRVVLKMPFADGTEDSEFIHNWKDAEQLS | |
| | | VETEVRFDNLGKRGQDAMNSYMNMANCPSTSAVRAKRSPG | |
| | | KICLSKINWKNVREKADALTKKVHADKEFMDKLSTHHQRG | |
| | | EAPSVEKTTALHNALLEHESFSALKGARASGKVGAAASTA | |
| | | AWGVAVAQAFTDPKADALTKTAATLSVVPGLGQALGIADG | |
| | | IKHENTEEIVVQSISLAGLLAAQAIPVVGEAVDFGLLVYQ | |
| | | LVETIVDLATHLSSAAANPPTEATDSVRPAVSLGLRAGWK | |
| | | TEEDAKLHIGSPYGMKFQRIVLSAEEGKEIPFVRAAVAVD | |
| | | SKFLKINGPRSFVVQNGIKTPMACFETEGNLAFCRPSRPI | |
| | | FLSSSSPATLHLSYVTNEHENGTIKNPTVDILGQRIVENK | |
| | | VITANKVSLVYKVDSSNTL | |
| 21 | Austwickia chelonae | YANDAVIADQSKTVDSFTSYHGAKPESFESVLAGIKKPES | WP_162873017.1 |
| | LK16-18 (CT2) | GSQGNHDPEWKGFYTTDNKHAAAGYTVSDESVMTGKAGGV | Y64-L603 |
| | | VKVTYPGKTRVLAVKPLSATELKTYLGLAADKPLIDQLNN | |
| | | KDFINKFGEGASRVVLQMPFADGTSDVEYIHNWEDATQLQ | |
| | | VATEVRFDNLGKRGQDEMNRYMNLANCPSVSAVRVKRNPA | |
| | | KLCLSKVKWEQVREKSKKIIDNVKDNPEFMKKLSAHHERG | |
| | | SAPTTEKITALHNELLDHESFSALKGARSSAGTAATAASA | |
| | | AAWGLAVAQAFTNPKADDLTKATAVLSAVPGLGQALGIAD | |
| | | GIKHHNTEEIVVQSISLTALIAAQAIPVVGELVDFGLLAY | |
| | | QLVESIIDLTRQLSVITANPPTEVTHSSKLAKSNGLLAGW | |
| | | KTDQDGVLSLGAPHGMKTQLISMSAEKGQEIPFTGAMIAV | |
| | | KKDLLTVNTPRVFVVQNGIRIPLRCSAAGETLTFCRPNHP | |
| | | VWLSEQHQATLHLSYVTQENESEKIDNPTVDILGQKIVDN | |
| | | KVSTANKVSLVYEVDRSNKL | |
| 35 | Austwickia sp. TVS | GAFASPISPAIDKEKSKVVENFTSYHGTKHQYIENVLKGI | WP_219106995.1 |
| | 96-490-7B | NKPETGSQGNHDPDWKGFYTTDSKYAAAGYTPDEENPMSG | G27-N663 |
| | (CT3) | KAGGVVKVSYTGKTKILALKDMPAGKIKGHLGLDPNKPLT | |
| | | EQIKDQEFINKYGEGADRVVLQMPFAEGSSEVEYINNWDN | |
| | | AKDLKVEPEVRFDQHGKRGQDAMYEYMALASCESAGKAKR | |
| | | SVSKTCLSNIKWERVRQKSEELIKKLKDDENVKKAVQERK | |
| | | EGVKPTSDDLQNLHKALIDHESFKELKSVHSNGVKAMDAA | |
| | | NAALWGANVARVFSDSKSDGLEKATAALAAVPGLGQVMGV | |
| | | ADGVTHHNTEEVVVQSVALAGFIAAQAIPVVGEIVDIGVL | |
| | | AYQFVEGVIDLSRQMMTSNARGIAEKRQVVDIKDANGLSG | |
| | | GWLTPQDSKIHLPPHYGTKTQRLAVSANGDHNLPITGFKT | |
| | | SINPKFFNINESGSFIVQNGLKVPMNCSEEASKTEAVCIP | |
| | | VHPIWLTDKNNAIIHVSYQTKSPEGSRIDNPRLEVWGQIM | |
| | | HTEEGPKTNTINLGYSVDQEMPCYSSIKFENGGDYVGRLA | |
| | | VNSKDGKVTHSKMSGMRDKDKVDLSAFNSGEVLTTEISPT | |
| | | GGGAPTPGPSIKYCSDGGIEASIKSWGTKDKAFLGEN | |

Translocation Domains

| | | | Source |
|---|---|---|---|
| | | | Sequence or |
| | | | GenBank |
| | | | Accession |
| | | | (full-length |
| SEQ | | | sequence with |
| ID NO: | Species | Translocation Domain Sequence | coordinates) |
| 3 | Austwickia chelonae | LSKINWKNVREKADALTKKVHADKEFMDKLSTHHQRGEAP | SEQ ID NO: 2 |
| | (CT1-T) | SVEKTTALHNALLEHESFSALKGARASGKVGAAASTAAWG | L204-N378 |
| | | VAVAQAFTDPKADALTKTAATLSVVPGLGQALGIADGIKH | |
| | | ENTEEIVVQSISLAGLLAAQAIPVVGEAVDFGLLVYQLVE | |
| | | TIVDLATHLSSAAAN | |
| 4 | Streptosporangium | LPVDWDKVEEKAKATAKKVAQDAEHVERLPKRSPKGPTWG | PSJ28985.1 |
| | nondiastaticum | EAHSTAELTHAKVREVSGAHVAASAAGVGTWVYGMAKTFS | L231-D394 |
| | | DKNATTLDKVAVTGAVVPGLGQALGIADGIQHGDPEAIAV | |
| | | NAVALAALAAAQVVPVVGEVVDAVLLTEQLVEVLVDVFRT | |
| | | ATAD | |
| 5 | Streptomyces sp.TLI | IPAASWEAIEHRSKEMASAVARDTEYTKSLPNRHPKGPTW | SDT83331.1 |
| | 053 | SEAHTTSTSTHARVSAKSGAHVAVGAFAVGSWIYGMSETF | 1198-E362 |
| | | ANKNVTTLDKAAATVAIVPGIGHALGIAAALEHHDIEGVV | |
| | | VNAISIAALAAAQVVPVVGEIVDAALLAEQLVEVLVHVFR | |
| | | ASTTE | |
| 6 | Streptomyces sp. | LSEMDGEKVTKGAQSVADTLKGHGNLQPLVKRTRAEPRTQ | WP_160159328.1 |
| | SLBN-118 | QYLPPTRQQIDDAISAAQRVHPGVDTAMKHLGSAQALAGF | L631-G801 |
| | | ALDVSQGEPLRAAADVTAMLPRIGPGLGNALGLADSIVHD | |
| | | DPEGIAVNAIGLGAFIVAQAIPVIGELADLGFAAYALIET | |
| | | LKSMFAGPTLG | |
| 7 | Streptomyces sp. | LPVDWDKVEEKAKATAKKVAQDAEHVERLPKRNPKGPTWG | WP_168096531.1 |
| | AA8 | EAHSTAELTHAKVRAVSGAHVAATAAGVGTWVYGMAKTFS | L241-D404 |
| | | DKDATTLDKVAVTGAVVPGLGQALGIADGIQHGDPEAVAV | |
| | | NAVALAALAAAQVVPVVGEVVDAALLTEQLVEVLVDVFRT | |
| | | ATAD | |
| 8 | Streptomyces | LPVDWDKVEEKAKATAKKVAQDAEHVERLPKRNPKGPTWG | WP_078659863.1 |
| | roseoverticillatus | EAHSTAELTHAKVRAVSGAHVAATAAGVGTWVYGMAKTFS | L231-D394 |
| | | DKDATTLDKVAVTGAVVPGLGQALGIADGIQHGDPEAVAV | |
| | | NAVALAALAAAQVVPVVGEVVDAALLTEQLVEVLVDVFRT | |
| | | ATAD | |
| 9 | Streptomyces | ELAKDKARDILSEAGNEVSLPQRDSDGLSKQEITATAEAT | JZ58907.1 |
| | piniterrae | RSKLGTGVHGAVSAAMVADWAHDVARTFADPKATKLDKAA | E225-A383 |
| | | AVTAIAPVIGQAVNIADGIQHHDKKTIVVNSLVLAAVVAA | |
| | | QAVPAVGEVVDAAIVADFVVEKLVGWFTPTAKPGPEHVA | |
| 10 | Streptomyces | LPVDWDKVEEKAKATAKKVAQDAEHAERLPKRNPKGPTWG | WP_079110321.1 |
| | MBT76 | EAHSTAELTHAKVREVSGTHVAATAAGVGTWVYGMAKTFS | L181-D344 |
| | | DKDATTLDKVAVTGAVVPGLGQALGIADGIQHGDPEAVAV | |
| | | NAVALAALAAAQVVPVVGEVVDAVLLTEQLVEVLVDVFRA | |
| | | ATAD | |
| 11 | Streptomyces klenkii | LPVDWDKVEEKAKATAKKVAQDAEHVEKLPKRSPKGPTWG | WP_120757473.1 |
| | | EAHSTAELSHAKVREVSGAHVAASAAGVGTWVYGMAKTFS | L227-D390 |
| | | DKDATTLDKVAVTGAVVPGLGQALGIADGIQHGDPEAIAV | |
| | | NAVALAALAAAQVVPVVGEVVDAVLLTEQLVEVLVDVFRT | |
| | | ATAD | |
| 12 | Streptomyces | GINVAAYKADIEKLIKDVYEDPNFSDLKNRTGGPQKDKDT | WP_095582082.1 |
| | albireticuli | LKGYYERLKPKVETLRPLKAGVSSAVGAAGAISWAIGVAD | G232-Q399 |
| | | AFTSENVSSFDKAAAVTAIVPGLGECVGIANAIDKRDPEG | |
| | | LIINTISMAALMASAAVPVLAPIGVALDAGLAAAQGVATV | |
| | | LEYLEIGQ | |
| 13 | Streptacidiphilus | KVNWKDAYDRSMNIAQDIDGSAEFRATAPARPAAGKAMPE | WP_133259917.1 |
| | pinicola | ADVTRLVSTSEDFLEKSTKTSGVKKALKAINNQKMISWGS | K246-1421 |
| | | LLSNALANSHTWSDKNATNLDKAYAVVGGVPVLGEVIGIA | |
| | | SGIDKQDAESIAVNTLSLVGIVAATVCPPLGATVEFVMIG | |
| | | YTAIKLMLSWFTVETI | |
| 14 | Seinonella | LDWEKIKTESQRIVKQIIEEHPELQSHSKNAVTDKEKLQK | WP_073156187.1 |
| | peptonophila | IYNDYAPKIDKLSSLKEGVSRATTALNIASWAAGLAETFS | L252-T416 |
| | | NKNADGLDKAAAVTAIIPGLGQAVGIANGIEKHDGEAIAI | |
| | | NSIALSALVVAQAIPIVGEIADVVGAGLILAGGLAQLIQS | |
| | | VSPDT | |
| 15 | Longimycelium | ERKTLDKLDRKKIETRAKQVISKLAQDGDLRRELPRRTTT | WP_189053160.1 |
| | tulufanense | GHAHEEVRGVLAKSRGALHEIHGAATTVAMPLATVAWVED | E249-W426 |
| | | MARVFREKNATTLDKAATVSEIVPVAGQVLGMADGIAHRD | |
| | | AETVAVNAVVLAAIAVSQAVPVVGELVDLGLTAYAVVDVV | |
| | | VRLFGPAREVPITQESYW | |
| 16 | Austwickia sp. TVS | LSNIKWERVRQKSEELIKKLKDDENVKKAVQERKEGVKPT | WP_219106995.1 |
| | 96-490-7B | SDDLQNLHKALIDHESFKELKSVHSNGVKAMDAANAALWG | L233-R407 |
| | (CT3-T) | ANVARVFSDSKSDGLEKATAALAAVPGLGQVMGVADGVTH | |
| | | HNTEEVVVQSVALAGFIAAQAIPVVGEIVDIGVLAYQFVE | |
| | | GVIDLSRQMMTSNAR | |
| 17 | Austwickia chelonae | LSKVKWEQVREKSKKIIDNVKDNPEFMKKLSAHHERGSAP | WP_162873017.1 |
| | LK16-18 | TTEKITALHNELLDHESFSALKGARSSAGTAATAASAAAW | L267-N442 |
| | (CT2-T) | GLAVAQAFTNPKADDLTKATAVLSAVPGLGQALGIADGIK | |
| | | HHNTEEIVVQSISLTALIAAQAIPVVGELVDFGLLAYQLV | |
| | | ESIIDLTRQLSVITAN | |
| 36 | Klebsiella aerogenes | GLNWGSIREKAVDKAKSVTSDKKFINSLPERGKNSPANKN | EIZ2913133.1 |
| | | DINTIYESSANHSTLKGGSEVIHKASLLLWVANTADVLTS | G211-R376 |
| | | NSSSTLDKATAIIGIVPGIGDAFSLADSIEHDDVEGIITS | |
| | | SIALAAFTVAQAVPVVGELVDLALLTEAAISSLVNVISNY | |
| | | IQSTQR | |
| 37 | Streptomyces sp. | ADLDWEKVRSRAKETARQVAQDSEHVSGLPERRAEGLTKS | MCH0551590.1 |
| | MUM 178J | EAHAVAERTHAKVSEVSGTRVAATAFGVGTWAYGMAQTFT | A229-A392 |
| | | DQNATTLDKVAVTTAIVPGVGNALGIADGIEHHDPEAVAV | |
| | | NAVALAALVAAQAVPVVGELVDTVLLTEQIVETFVNIFQR | |
| | | ATAA | |
| 38 | Crossiella cryophila | ETADSLGELFHQGMTIAGEAVAELDLEEALKSQKVAKGDA | MBB4677777.1 |
| | | RAVVAGKVAAVGKQVGKSSALAKFKEVLKPSKGVIALWAV | E56-C217 |
| | | SALYTLAQEDVSTVDKAEAVLAIVPILGPLFSFGNSYRKE | |
| | | DVEGMVISTVALAATLIAFLCPPVGAGIGVGLAVYQVVRA | |
| | | FC | |
| 39 | Allokutzneria sp. | SLVKGKTDAQAVSAVKQLFENSKTVATSLVADSDMADAIK | WP_143261759.1 |
| | NRRL B-24872 | DAKKAAKVGVAPPVDKVKKVTDPLVEKALKSKSFANTKAG | S60-C220 |
| | | KYLKNAQPSKAGTALWAATTVATFLDGSSSASDKAVAAVG | |
| | | IIPVVGQLASLIHTTIKDPSDVEGIVTGTIGLAAAVITIA | |
| | | C | |
| 40 | Allokutzneria albata | KAIGATRESARKFVANILSKSEKATTTLAASNDYKSSMKGF | WP_156051914.1 |
| | | SESFAKLRKAAAETKKPIPTADIKKLLTPHLDKVSTVKDFK | K78-G240 |
| | | KYFTNLKPSQGSAATWLVNLAATVANEESSKADVAVAAAGI | |
| | | IPVVGQVAAIANSVSQGDVEGTVTSAIALAANIAIAVGAG | |
| 41 | Streptomyces sp. | CDIGVRDHAEVARKHQERLRAHGDDVEYVTTLDEFEKNPK | WP_199893204.1 |
| | AV19 | SLLSPLRETNGVLNDAAAAELGAKDFEAFSTRVTEALGRD | C260-P481 |
| | | LRAVHGLSTDTTLLAKVARQAGRAAELGAKALPYVGIAAT | |
| | | GYAIAEDVKAGDYANLAFDSVAEGLQVAMVAQPELVPLLE | |
| | | PVLFAEQLAQIVYDEIAGWFKRQEQLDHDRKQWDSAAEDL | |
| | | VEHRDGQWRDHLVGQALQKLEP | |
| 42 | Streptomyces sp. | CGWGDDGVAHTQRERLRAQGGDVEYVTSLEEFEKNPKALL | WP_147264604.1 |
| | NRBC_110611 | SPLRETNKVLNDAASAELGAKDFETFSARVGAAFSRDLRA | C231-P449 |
| | | VQGLTTDTGLLAGVGRQVGRAGDLGVKVLPYVGIAVTGYA | |
| | | VAEDAKAGDYANLAFDSVAEGLQVAMVAQPEVAPLLEPAL | |
| | | LAEQLAQLVYNEVAGWIAEQKQLEHDRAQWDGAVKDLVAH | |
| | | RDTQWRDRLVGQAVQELFP | |
| 43 | Streptomyces | CGIAAADDAKVARTHQERLRAQGADVEYVTSLEEFEKNPK | WP_209513619.1 |
| | syringium | ALMSPLRETSKVLNGAAAAELGAKDFEAFSTQVTEALGRD | C232-P453 |
| | | LKAVHGLTTDTGLLAEVGRRVGRAGELGVKILPYAGIAAT | |
| | | GYAVAEDAKAGDYGNLAFDSVAEGLQVAMVAQPELTPLLE | |
| | | PALLAEQLAQLVYDEVAGWIAEQKQLAHDRGQWDDAVKDL | |
| | | VAHRDTQWRNRLEGQALQELFP | |
| 44 | Pseudonocardiaceae | RIGKAALAKATAEAQADAQKILKDPSFKPPHPAPHQKLTD | RJQ69589.1 |
| | bacterium YIM PH | AELTQVANDLAKRVSALHPGARDLTGLGKGLAKALKGVIS | R276-F454 |
| | 21723 | GVTAGIQAAEIAESAAGSNSGDLEKVCSALGPIPIVGDIA | |
| | | GIVNAAAEGDAVEIVKNVLTLGLSMVSVAFPPLAPVADAV | |
| | | AFVLEVAVFLGKYLWGLLF | |
| 45 | Actinokineospora | SLSQDQLDQAVADAQATAHQVIDDPRSFELPELAKGQVLS | WP_143218892.1 |
| | bangkokensis | EQELAALANGVAQDVAAANSGGERAIVPAYVTRALSKAIP | S322-L515 |
| | | AIAEHGHDAFLKVSAVEGVLTEGNGALAKVTSVVGPVPVL | |
| | | GSLAGIVNEAVGISADDPPAAIAQKALEIAKNVAGVALTV | |
| | | MAIAFPPFAPVAALATAAWEVGAALGKYLLGELL | |
| 46 | Streptomyces | CETADGGDTAEEGKQPGEGRQATESDSTEGRNPQGEEGPA | MBF6055834.1 |
| | eurocidicus | EAGKPVADEEWVARVQRERLARHGSDVEYVTSVAELEKNP | C280-R449 |
| | | KAVLSPLRETNTTLNKARDKPLTAAEYEAFASKAAEAMSK | |
| | | DLKGVQAASTDSTVLAKVGAKLARGGSIAVKVGRPVLKRR | |
| | | LLRSSAASSR | |
| 47 | Streptomyces | GTGRQDGGFSSEGREGAVTKEDLKGDPQKVVELNRSRIAR | WP_169790908.1 |
| | pathocidini | HGADVDYVTSLDALAANPKAVLIPLRNSSQTLDAALGKSH | G270-A425 |
| | | LTVDEFDRFSAELTQSFGRDLQAVENLSTSEKLLSSIGKG | |
| | | ARAGGEGLLKVVPFAGVAASSAALAEDLKSGENGDA | |
| 48 | Streptomyces | SADIARTHRERLRAHGDDVEYVTSLAEFEKNPKALLSPLR | WP_157868472.1 |
| | caatingaensis | ETGKVLNDAGNAGLSAGDFETFSARVGEAFERDLRAAHEL | S241-A389 |
| | | TTDTGLLARVGAEAGRAAELGAKALPFVGIAATGYAVAED | |
| | | VKSGDYANLAFDSVAEGLQVAMTAQPEFA | |

Constructs and Modules

| SEQ | | | |
|---|---|---|---|
| ID NO: | Description | Sequence | Source |
| 18 | Vibrio vulnificus | GDKTKVVVDLAQIFTVQELKERAKVFAKPIGASYQGILDQ | WP_011081430.1 |
| | (RRSP) | LDLVHQAKGRDQIAASFELNKKINDYIAEHPTSGRNQALT | G3580-L4089 |
| | | QLKEQVTSALFIGKMQVAQAGIDAIAQTRPELAARIFMVA | |
| | | IEEANGKHVGLTDMMVRWANEDPYLAPKHGYKGETPSDLG | |
| | | FDAKYHVDLGEHYADFKQWLETSQSNGLLSKATLDESTKT | |
| | | VHLGYSYQELQDLTGAESVQMAFYFLKEAAKKADPISGDS | |
| | | AEMILLKKFADQSYLSQLDSDRMDQIEGIYRSSHETDIDA | |
| | | WDRRYSGTGYDELTNKLASATGVDEQLAVLLDDRKGLLIG | |
| | | EVHGSDVNGLRFVNEQMDALKKQGVTVIGLEHLRSDLAQP | |
| | | LIDRYLATGVMSSELSAMLKTKHLDVTLFENARANGMRIV | |
| | | ALDANSSARPNVQGTEHGLMYRAGAANNIAVEVLQNLPDG | |
| | | EKFVAIYGKAHLQSHKGIEGFVPGITHRLDLPALKVSDSN | |
| | | QFTVEQDDVSLRVVYDDVANKPKITFKGSL | |
| 19 | Her3 affibody | AEAKYAKEKYNAYYEIWQLPNLTKYQKAAFIGKLQDDPSQ | Nazari et al. |
| | (ZHer3:08699) | SSELLSEAKKLNDSQAPK | (2019) |
| 20 | A20 peptide | NAVPNLRGDLQVLAQKVART | DiCara et al. |
| | (A20FMDV2) | | (2007) |
| 22 | RRSP-DT-T- | GDKTKVVVDLAQIFTVQELKERAKVFAKPIGASYQGILDQ | n/a |
| | ZHer3-A20 | LDLVHQAKGRDQIAASFELNKKINDYIAEHPTSGRNQALT | |
| | Construct | QLKEQVTSALFIGKMQVAQAGIDAIAQTRPELAARIFMVA | |
| | | IEEANGKHVGLTDMMVRWANEDPYLAPKHGYKGETPSDLG | |
| | | FDAKYHVDLGEHYADFKQWLETSQSNGLLSKATLDESTKT | |
| | | VHLGYSYQELQDLTGAESVQMAFYFLKEAAKKADPISGDS | |
| | | AEMILLKKFADQSYLSQLDSDRMDQIEGIYRSSHETDIDA | |
| | | WDRRYSGTGYDELTNKLASATGVDEQLAVLLDDRKGLLIG | |
| | | EVHGSDVNGLRFVNEQMDALKKQGVTVIGLEHLRSDLAQP | |
| | | LIDRYLATGVMSSELSAMLKTKHLDVTLFENARANGMRIV | |
| | | ALDANSSARPNVQGTEHGLMYRAGAANNIAVEVLQNLPDG | |
| | | EKFVAIYGKAHLQSHKGIEGFVPGITHRLDLPALKVSDSN | |
| | | QFTVEQDDVSLRVVYDDVANKPKITFKGSLGGGGSGGGGS | |
| | | CAGNRVRRSVGSSLSCINLDWDVIRDKTKTKIESLKEHGP | |
| | | IKNKMSESPNKTVSEEKAKQYLEEFHQTALEHPELSELKT | |
| | | VTGTNPVFAGANYAAWAVNVAQVIDSETADNLEKTTAALS | |
| | | ILPGIGSVMGIADGAVHHNTEEIVAQSIALSSLMVAQAIP | |
| | | LVGELVDIGFAAYNFVESIINLFQVVHNSYNRPAYSPGHK | |
| | | TQPFGGGGSGGGGSAEAKYAKEKYNAYYEIWQLPNLTKYQ | |
| | | KAAFIGKLQDDPSQSSELLSEAKKLNDSQAPKGGGGSGGG | |
| | | GSNAVPNLRGDLQVLAQKVARTRQALVPRGSAWSHPQFEK | |
| 23 | RRSP-CT1-T- | GDKTKVVVDLAQIFTVQELKERAKVFAKPIGASYQGILDQ | n/a |
| | ZHer3-A20 | LDLVHQAKGRDQIAASFELNKKINDYIAEHPTSGRNQALT | |
| | Construct | QLKEQVTSALFIGKMQVAQAGIDAIAQTRPELAARIFMVA | |
| | | IEEANGKHVGLTDMMVRWANEDPYLAPKHGYKGETPSDLG | |
| | | FDAKYHVDLGEHYADFKQWLETSQSNGLLSKATLDESTKT | |
| | | VHLGYSYQELQDLTGAESVQMAFYFLKEAAKKADPISGDS | |
| | | AEMILLKKFADQSYLSQLDSDRMDQIEGIYRSSHETDIDA | |
| | | WDRRYSGTGYDELTNKLASATGVDEQLAVLLDDRKGLLIG | |
| | | EVHGSDVNGLRFVNEQMDALKKQGVTVIGLEHLRSDLAQP | |
| | | LIDRYLATGVMSSELSAMLKTKHLDVTLFENARANGMRIV | |
| | | ALDANSSARPNVQGTEHGLMYRAGAANNIAVEVLQNLPDG | |
| | | EKFVAIYGKAHLQSHKGIEGFVPGITHRLDLPALKVSDSN | |
| | | QFTVEQDDVSLRVVYDDVANKPKITFKGSLGGGGSGGGGS | |
| | | CAGNRVRRSVGSSLSCLSKINWKNVREKADALTKKVHADK | |
| | | EFMDKLSTHHQRGEAPSVEKTTALHNALLEHESFSALKGA | |
| | | RASGKVGAAASTAAWGVAVAQAFTDPKADALTKTAATLSV | |
| | | VPGLGQALGIADGIKHENTEEIVVQSISLAGLLAAQAIPV | |
| | | VGEAVDFGLLVYQLVETIVDLATHLSSAAANPAYSPGHKT | |
| | | QPFGGGGSGGGGSAEAKYAKEKYNAYYEIWQLPNLTKYQK | |
| | | AAFIGKLQDDPSQSSELLSEAKKLNDSQAPKGGGGSGGGG | |
| | | SNAVPNLRGDLQVLAQKVARTRQALVPRGSAWSHPQFEK | |
| 24 | RRSP-CT2-T- | GDKTKVVVDLAQIFTVQELKERAKVFAKPIGASYQGILDQ | n/a |
| | ZHer3-A20 | LDLVHQAKGRDQIAASFELNKKINDYIAEHPTSGRNQALT | |
| | Construct | QLKEQVTSALFIGKMQVAQAGIDAIAQTRPELAARIFMVA | |
| | | IEEANGKHVGLTDMMVRWANEDPYLAPKHGYKGETPSDLG | |
| | | FDAKYHVDLGEHYADFKQWLETSQSNGLLSKATLDESTKT | |
| | | VHLGYSYQELQDLTGAESVQMAFYFLKEAAKKADPISGDS | |
| | | AEMILLKKFADQSYLSQLDSDRMDQIEGIYRSSHETDIDA | |
| | | WDRRYSGTGYDELTNKLASATGVDEQLAVLLDDRKGLLIG | |
| | | EVHGSDVNGLRFVNEQMDALKKQGVTVIGLEHLRSDLAQP | |
| | | LIDRYLATGVMSSELSAMLKTKHLDVTLFENARANGMRIV | |
| | | ALDANSSARPNVQGTEHGLMYRAGAANNIAVEVLQNLPDG | |
| | | EKFVAIYGKAHLQSHKGIEGFVPGITHRLDLPALKVSDSN | |
| | | QFTVEQDDVSLRVVYDDVANKPKITFKGSLGGGGSGGGGS | |
| | | CAGNRVRRSVGSSLSCLSKVKWEQVREKSKKIIDNVKDNP | |
| | | EFMKKLSAHHERGSAPTTEKITALHNELLDHESFSALKGA | |
| | | RSSAGTAATAASAAAWGLAVAQAFTNPKADDLTKATAVLS | |
| | | AVPGLGQALGIADGIKHHNTEEIVVQSISLTALIAAQAIP | |
| | | VVGELVDFGLLAYQLVESIIDLTRQLSVITANPAYSPGHK | |
| | | TQPFGGGGSGGGGSAEAKYAKEKYNAYYEIWQLPNLTKYQ | |
| | | KAAFIGKLQDDPSQSSELLSEAKKLNDSQAPKGGGGSGGG | |
| | | GSNAVPNLRGDLQVLAQKVARTRQALVPRGSAWSHPQFEK | |
| 25 | RRSP-CT3-T- | GDKTKVVVDLAQIFTVQELKERAKVFAKPIGASYQGILDQ | n/a |
| | ZHer3-A20 | LDLVHQAKGRDQIAASFELNKKINDYIAEHPTSGRNQALT | |
| | Construct | QLKEQVTSALFIGKMQVAQAGIDAIAQTRPELAARIFMVA | |
| | | IEEANGKHVGLTDMMVRWANEDPYLAPKHGYKGETPSDLG | |
| | | FDAKYHVDLGEHYADFKQWLETSQSNGLLSKATLDESTKT | |
| | | VHLGYSYQELQDLTGAESVQMAFYFLKEAAKKADPISGDS | |
| | | AEMILLKKFADQSYLSQLDSDRMDQIEGIYRSSHETDIDA | |
| | | WDRRYSGTGYDELTNKLASATGVDEQLAVLLDDRKGLLIG | |
| | | EVHGSDVNGLRFVNEQMDALKKQGVTVIGLEHLRSDLAQP | |
| | | LIDRYLATGVMSSELSAMLKTKHLDVTLFENARANGMRIV | |
| | | ALDANSSARPNVQGTEHGLMYRAGAANNIAVEVLQNLPDG | |
| | | EKFVAIYGKAHLQSHKGIEGFVPGITHRLDLPALKVSDSN | |
| | | QFTVEQDDVSLRVVYDDVANKPKITFKGSLGGGGSGGGGS | |
| | | CAGNRVRRSVGSSLSCLSNIKWERVRQKSEELIKKLKDDE | |
| | | NVKKAVQERKEGVKPTSDDLQNLHKALIDHESFKELKSVH | |
| | | SNGVKAMDAANAALWGANVARVFSDSKSDGLEKATAALAA | |
| | | VPGLGQVMGVADGVTHHNTEEVVVQSVALAGFIAAQAIPV | |
| | | VGEIVDIGVLAYQFVEGVIDLSRQMMTSNARPAYSPGHKT | |
| | | QPFGGGGSGGGGSAEAKYAKEKYNAYYEIWQLPNLTKYQK | |
| | | AAFIGKLQDDPSQSSELLSEAKKLNDSQAPKGGGGSGGGG | |
| | | SNAVPNLRGDLQVLAQKVARTRQALVPRGSAWSHPQFEK | |
| 26 | RRSP-T (S. | GDKTKVVVDLAQIFTVQELKERAKVFAKPIGASYQGILD | n/a |
| | klenkii)-ZHer3-A20 | QLDLVHQAKGRDQIAASFELNKKINDYIAEHPTSGRNQA | |
| | Construct | LTQLKEQVTSALFIGKMQVAQAGIDAIAQTRPELAARIF | |
| | | MVAIEEANGKHVGLTDMMVRWANEDPYLAPKHGYKGETP | |
| | | SDLGFDAKYHVDLGEHYADFKQWLETSQSNGLLSKATLD | |
| | | ESTKTVHLGYSYQELQDLTGAESVQMAFYFLKEAAKKAD | |
| | | PISGDSAEMILLKKFADQSYLSQLDSDRMDQIEGIYRSS | |
| | | HETDIDAWDRRYSGTGYDELTNKLASATGVDEQLAVLLD | |
| | | DRKGLLIGEVHGSDVNGLRFVNEQMDALKKQGVTVIGLE | |
| | | HLRSDLAQPLIDRYLATGVMSSELSAMLKTKHLDVTLFE | |
| | | NARANGMRIVALDANSSARPNVQGTEHGLMYRAGAANNI | |
| | | AVEVLQNLPDGEKFVAIYGKAHLQSHKGIEGFVPGITHR | |
| | | LDLPALKVSDSNQFTVEQDDVSLRVVYDDVANKPKITFK | |
| | | GSLGGGGSGGGGSCAGNRVRRSVGSSLSCLPVDWDKVEE | |
| | | KAKATAKKVAQDAEHVEKLPKRSPKGPTWGEAHSTAELS | |
| | | HAKVREVSGAHVAASAAGVGTWVYGMAKTFSDKDATTLD | |
| | | KVAVTGAVVPGLGQALGIADGIQHGDPEAIAVNAVALAA | |
| | | LAAAQVVPVVGEVVDAVLLTEQLVEVLVDVFRTATADPA | |
| | | YSPGHKTQPFGGGGSGGGGSAEAKYAKEKYNAYYEIWQL | |
| | | PNLTKYQKAAFIGKLQDDPSQSSELLSEAKKLNDSQAPK | |
| | | GGGGSGGGGSNAVPNLRGDLQVLAQKVARTRQALVPRGS | |
| | | AWSHPQFEK | |
| 27 | RRSP-T (S. sp | GDKTKVVVDLAQIFTVQELKERAKVFAKPIGASYQGILDQ | n/a |
| | TLI053)-ZHer3-A20 | LDLVHQAKGRDQIAASFELNKKINDYIAEHPTSGRNQALT | |
| | Construct | QLKEQVTSALFIGKMQVAQAGIDAIAQTRPELAARIFMVA | |
| | | IEEANGKHVGLTDMMVRWANEDPYLAPKHGYKGETPSDLG | |
| | | FDAKYHVDLGEHYADFKQWLETSQSNGLLSKATLDESTKT | |
| | | VHLGYSYQELQDLTGAESVQMAFYFLKEAAKKADPISGDS | |
| | | AEMILLKKFADQSYLSQLDSDRMDQIEGIYRSSHETDIDA | |
| | | WDRRYSGTGYDELTNKLASATGVDEQLAVLLDDRKGLLIG | |
| | | EVHGSDVNGLRFVNEQMDALKKQGVTVIGLEHLRSDLAQP | |
| | | LIDRYLATGVMSSELSAMLKTKHLDVTLFENARANGMRIV | |
| | | ALDANSSARPNVQGTEHGLMYRAGAANNIAVEVLQNLPDG | |
| | | EKFVAIYGKAHLQSHKGIEGFVPGITHRLDLPALKVSDSN | |
| | | QFTVEQDDVSLRVVYDDVANKPKITFKGSLGGGGSGGGGS | |
| | | CAGNRVRRSVGSSLSCIPAASWEAIEHRSKEMASAVARDT | |
| | | EYTKSLPNRHPKGPTWSEAHTTSTSTHARVSAKSGAHVAV | |
| | | GAFAVGSWIYGMSETFANKNVTTLDKAAATVAIVPGIGHA | |
| | | LGIAAALEHHDIEGVVVNAISIAALAAAQVVPVVGEIVDA | |
| | | ALLAEQLVEVLVHVFRASTTEPAYSPGHKTQPFGGGGSGG | |
| | | GGSAEAKYAKEKYNAYYEIWQLPNLTKYQKAAFIGKLQDD | |
| | | PSQSSELLSEAKKLNDSQAPKGGGGSGGGGSNAVPNLRGD | |
| | | LQVLAQKVARTRQALVPRGSAWSHPQFEK | |
| 28 | RRSP-T (L. | GDKTKVVVDLAQIFTVQELKERAKVFAKPIGASYQGILDQ | n/a |
| | tulufanense)- | LDLVHQAKGRDQIAASFELNKKINDYIAEHPTSGRNQALT | |
| | ZHer3-A20 | QLKEQVTSALFIGKMQVAQAGIDAIAQTRPELAARIFMVA | |
| | Construct | IEEANGKHVGLTDMMVRWANEDPYLAPKHGYKGETPSDLG | |
| | | FDAKYHVDLGEHYADFKQWLETSQSNGLLSKATLDESTKT | |
| | | VHLGYSYQELQDLTGAESVQMAFYFLKEAAKKADPISGDS | |
| | | AEMILLKKFADQSYLSQLDSDRMDQIEGIYRSSHETDIDA | |
| | | WDRRYSGTGYDELTNKLASATGVDEQLAVLLDDRKGLLIG | |
| | | EVHGSDVNGLRFVNEQMDALKKQGVTVIGLEHLRSDLAQP | |
| | | LIDRYLATGVMSSELSAMLKTKHLDVTLFENARANGMRIV | |
| | | ALDANSSARPNVQGTEHGLMYRAGAANNIAVEVLQNLPDG | |
| | | EKFVAIYGKAHLQSHKGIEGFVPGITHRLDLPALKVSDSN | |
| | | QFTVEQDDVSLRVVYDDVANKPKITFKGSLGGGGSGGGGS | |
| | | CAGNRVRRSVGSSLSCERKTLDKLDRKKIETRAKQVISKL | |
| | | AQDGDLRRELPRRTTTGHAHEEVRGVLAKSRGALHEIHGA | |
| | | ATTVAMPLATVAWVEDMARVFREKNATTLDKAATVSEIVP | |
| | | VAGQVLGMADGIAHRDAETVAVNAVVLAAIAVSQAVPVVG | |
| | | ELVDLGLTAYAVVDVVVRLFGPAREVPITQESYWPAYSPG | |
| | | HKTQPFGGGGSGGGGSAEAKYAKEKYNAYYEIWQLPNLTK | |
| | | YQKAAFIGKLQDDPSQSSELLSEAKKLNDSQAPKGGGGSG | |
| | | GGGSNAVPNLRGDLQVLAQKVARTRQALVPRGSAWSHPQF | |
| | | EK | |
| 29 | RRSP-T (S. | GDKTKVVVDLAQIFTVQELKERAKVFAKPIGASYQGILDQ | n/a |
| | piniterrae)-ZHer3- | LDLVHQAKGRDQIAASFELNKKINDYIAEHPTSGRNQALT | |
| | A20 Construct | QLKEQVTSALFIGKMQVAQAGIDAIAQTRPELAARIFMVA | |
| | | IEEANGKHVGLTDMMVRWANEDPYLAPKHGYKGETPSDLG | |
| | | FDAKYHVDLGEHYADFKQWLETSQSNGLLSKATLDESTKT | |
| | | VHLGYSYQELQDLTGAESVQMAFYFLKEAAKKADPISGDS | |
| | | AEMILLKKFADQSYLSQLDSDRMDQIEGIYRSSHETDIDA | |
| | | WDRRYSGTGYDELTNKLASATGVDEQLAVLLDDRKGLLIG | |
| | | EVHGSDVNGLRFVNEQMDALKKQGVTVIGLEHLRSDLAQP | |
| | | LIDRYLATGVMSSELSAMLKTKHLDVTLFENARANGMRIV | |
| | | ALDANSSARPNVQGTEHGLMYRAGAANNIAVEVLQNLPDG | |
| | | EKFVAIYGKAHLQSHKGIEGFVPGITHRLDLPALKVSDSN | |
| | | QFTVEQDDVSLRVVYDDVANKPKITFKGSLGGGGSGGGGS | |
| | | CAGNRVRRSVGSSLSCELAKDKARDILSEAGNEVSLPQRD | |
| | | SDGLSKQEITATAEATRSKLGTGVHGAVSAAMVADWAHDV | |
| | | ARTFADPKATKLDKAAAVTAIAPVIGQAVNIADGIQHHDK | |
| | | KTIVVNSLVLAAVVAAQAVPAVGEVVDAAIVADFVVEKLV | |
| | | GWFTPTAKPGPEHVAPAYSPGHKTQPFGGGGSGGGGSAEA | |
| | | KYAKEKYNAYYEIWQLPNLTKYQKAAFIGKLQDDPSQSSE | |
| | | LLSEAKKLNDSQAPKGGGGSGGGGSNAVPNLRGDLQVLAQ | |
| | | KVARTRQALVPRGSAWSHPQFEK | |
| 30 | RRSP-T (S. | GDKTKVVVDLAQIFTVQELKERAKVFAKPIGASYQGILDQ | n/a |
| | pinicola)-ZHer3- | LDLVHQAKGRDQIAASFELNKKINDYIAEHPTSGRNQALT | |
| | A20 Construct | QLKEQVTSALFIGKMQVAQAGIDAIAQTRPELAARIFMVA | |
| | | IEEANGKHVGLTDMMVRWANEDPYLAPKHGYKGETPSDLG | |
| | | FDAKYHVDLGEHYADFKQWLETSQSNGLLSKATLDESTKT | |
| | | VHLGYSYQELQDLTGAESVQMAFYFLKEAAKKADPISGDS | |
| | | AEMILLKKFADQSYLSQLDSDRMDQIEGIYRSSHETDIDA | |
| | | WDRRYSGTGYDELTNKLASATGVDEQLAVLLDDRKGLLIG | |
| | | EVHGSDVNGLRFVNEQMDALKKQGVTVIGLEHLRSDLAQP | |
| | | LIDRYLATGVMSSELSAMLKTKHLDVTLFENARANGMRIV | |
| | | ALDANSSARPNVQGTEHGLMYRAGAANNIAVEVLQNLPDG | |
| | | EKFVAIYGKAHLQSHKGIEGFVPGITHRLDLPALKVSDSN | |
| | | QFTVEQDDVSLRVVYDDVANKPKITFKGSLGGGGSGGGGS | |
| | | CAGNRVRRSVGSSLSCKVNWKDAYDRSMNIAQDIDGSAEF | |
| | | RATAPARPAAGKAMPEADVTRLVSTSEDFLEKSTKTSGVK | |
| | | KALKAINNQKMISWGSLLSNALANSHTWSDKNATNLDKAY | |
| | | AVVGGVPVLGEVIGIASGIDKQDAESIAVNTLSLVGIVAA | |
| | | TVCPPLGATVEFVMIGYTAIKLMLSWFTVETIPAYSPGHK | |
| | | TQPFGGGGSGGGGSAEAKYAKEKYNAYYEIWQLPNLTKYQ | |
| | | KAAFIGKLQDDPSQSSELLSEAKKLNDSQAPKGGGGSGGG | |
| | | GSNAVPNLRGDLQVLAQKVARTRQALVPRGSAWSHPQFEK | |
| 31 | DT1-389-ZHer3-A20 | GADDVVDSSKSFVMENFSSYHGTKPGYVDSIQKGIQKPKS | n/a |
| | Construct | GTQGNYDDDWKGFYSTDNKYDAAGYSVDNENPLSGKAGGV | |
| | | VKVTYPGLTKVLALKVDNAETIKKELGLSLTEPLMEQVGT | |
| | | EEFIKRFGDGASRVVLSLPFAEGSSSVEYINNWEQAKALS | |
| | | VELEINFETRGKRGQDAMYEYMAQACAGNRVRRSVGSSLS | |
| | | CINLDWDVIRDKTKTKIESLKEHGPIKNKMSESPNKTVSE | |
| | | EKAKQYLEEFHQTALEHPELSELKTVTGTNPVFAGANYAA | |
| | | WAVNVAQVIDSETADNLEKTTAALSILPGIGSVMGIADGA | |
| | | VHHNTEEIVAQSIALSSLMVAQAIPLVGELVDIGFAAYNE | |
| | | VESIINLFQVVHNSYNRPAYSPGHKTQPFGGGGSGGGGSA | |
| | | EAKYAKEKYNAYYEIWQLPNLTKYQKAAFIGKLQDDPSQS | |
| | | SELLSEAKKLNDSQAPKGGGGSGGGGSNAVPNLRGDLQVL | |
| | | AQKVARTRQALVPRGSAWSHPQFEK | |
| 32 | CT21-391-ZHer3-A20 | YANDAVIADQSKTVDSFTSYHGAKPESFESVLAGIKKPES | n/a |
| | Construct | GSQGNHDPEWKGFYTTDNKHAAAGYTVSDESVMTGKAGGV | |
| | | VKVTYPGKTRVLAVKPLSATELKTYLGLAADKPLIDQLNN | |
| | | KDFINKFGEGASRVVLQMPFADGTSDVEYIHNWEDATQLQ | |
| | | VATEVRFDNLGKRGQDEMNRYMNLANCPSVSAVRVKRNPA | |
| | | KLCLSKVKWEQVREKSKKIIDNVKDNPEFMKKLSAHHERG | |
| | | SAPTTEKITALHNELLDHESFSALKGARSSAGTAATAASA | |
| | | AAWGLAVAQAFTNPKADDLTKATAVLSAVPGLGQALGIAD | |
| | | GIKHHNTEEIVVQSISLTALIAAQAIPVVGELVDFGLLAY | |
| | | QLVESIIDLTRQLSVITANPPTEVTHSSKLAGGGGSGGGG | |
| | | SAEAKYAKEKYNAYYEIWQLPNLTKYQKAAFIGKLQDDPS | |
| | | QSSELLSEAKKLNDSQAPKGGGGSGGGGSNAVPNLRGDLQ | |
| | | VLAQKVARTRQALVPRGSAWSHPQFEK | |
| 33 | Linker from C. | CAGNRVRRSVGSSLSC | n/a |
| | diphtheriae | | |
| | containing furin | | |
| | recognition site (with | | |
| | bracketing cysteines) | | |
| 34 | Linker after T | PAYSPGHKTQPF | n/a |
| | domain, from C. | | |
| | diphtheriae | | |

### REFERENCES

Orrell, K. E., Mansfield, M. J., Doxey, A. C. & Melnyk, R. A. The C. difficile toxin B membrane translocation machinery is an evolutionarily conserved protein delivery apparatus. Nature Communications 11, 1-11 (2020).
Park, M. et al. Intracellular Delivery of Human Purine Nucleoside Phosphorylase by Engineered Diphtheria Toxin Rescues Function in Target Cells. Molecular Pharmaceutics 15, 5217-5226 (2018).
Nazari, M.; Zamani Koukhaloo, S.; Mousavi, S.; Minai-Tehrani, A.; Emamzadeh, R.; Cheraghi, R. Development of a ZHER3-Affibody-Targeted Nano-Vector for Gene Delivery to HER3-Overexpressed Breast Cancer Cells. Macromol. Biosci. 2019, 19 (11).
DiCara, D.; Rapisarda, C.; Sutcliffe, J. L.; Violette, S. M.; Weinreb, P. H.; Hart, I. R.; Howard, M. J.; Marshall, J. F. Structure-Function Analysis of Arg-Gly-Asp Helix Motifs in Avβ6 Integrin Ligands. J. Biol. Chem. 2007, 282 (13), 9657-9665.
Sugiman-Marangos, S. N., Gill, S. K., Mansfield, M. J. et al. Structures of distant diphtheria toxin homologs reveal functional determinants of an evolutionarily conserved toxin scaffold. Commun Biol 5, 375 (2022).

In the preceding description, for purposes of explanation, numerous details are set forth in order to provide a thorough understanding of the embodiments. However, it will be apparent to one skilled in the art that these specific details are not required. In other instances, well-known electrical structures and circuits are shown in block diagram form in order not to obscure the understanding. For example, specific details are not provided as to whether the embodiments described herein are implemented as a software routine, hardware circuit, firmware, or a combination thereof.

The above-described embodiments are intended to be examples only. Alterations, modifications and variations can be effected to the particular embodiments by those of skill in the art. The scope of the claims should not be limited by the particular embodiments set forth herein, but should be construed in a manner consistent with the specification as a whole.

1. A recombinant polypeptide of general formula (I): 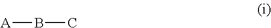
wherein:
A is a cargo molecule;
B is a translocation polypeptide comprising:
a) a translocation domain from:
i. the Austwickia chelonae protein of SEQ ID NO: 2,
ii. the Streptosporangium nondiastaticum protein of GenBank Accession PSJ28985.1,
iii. the Streptomyces sp. TLI 053 protein of GenBank Accession SDT83331.1,
iv. the Streptomyces sp. SLBN-118 protein of GenBank Accession WP_160159328.1,
v. the Streptomyces sp. AA8 protein of GenBank Accession WP_168096531.1,
vi. the Streptomyces roseoverticillatus protein of GenBank Accession WP_078659863.1,
vii. the Streptomyces piniterrae protein of GenBank Accession JZ58907.1,
viii. the Streptomyces MBT76 protein of GenBank Accession WP_079110321.1,
ix. the Streptomyces klenkii protein of GenBank Accession WP_120757473.1,
x. the Streptomyces albireticuli protein of GenBank Accession WP_095582082.1,
xi. the Streptacidiphilus pinicola protein of GenBank Accession WP_133259917.1,
xii. the Seinonella peptonophila protein of GenBank Accession WP_073156187.1,
xiii. the Longimycelium tulufanense protein of GenBank Accession WP_189053160.1,
xiv. the Austwickia sp. TVS 96-490-7B protein of GenBank Accession WP_219106995.1,
xv. the Austwickia chelonae LK16-18 protein of GenBank Accession WP_162873017.1;
xvi. the Klebsiella aerogenes protein of GenBank Accession EIZ2913133.1,
xvii. the Streptomyces sp. MUM 178J protein of GenBank Accession MCH0551590.1,
xviii. the Crossiella cryophila protein of GenBank Accession MBB4677777.1,
xix. the Allokutzneria sp. NRRL B-24872 protein of GenBank Accession WP_143261759.1,
xx. the Allokutzneria albata protein of GenBank Accession WP_156051914.1,
xxi. the Streptomyces sp. AV19 protein of GenBank Accession WP_199893204.1,
xxii. the Streptomyces sp. NRBC_110611 protein of GenBank Accession WP_147264604.1,
xxiii. the Streptomyces syringium protein of GenBank Accession WP_209513619.1,
xxiv. the Pseudonocardiaceae bacterium YIM PH 21723 protein of GenBank Accession RJQ69589.1,
xxv. the Actinokineospora bangkokensis protein of GenBank Accession WP_143218892.1,
xxvi. the Streptomyces eurocidicus protein of GenBank Accession MBF6055834.1,
xxvii. the Streptomyces pathocidini protein of GenBank Accession WP_169790908.1, or
xxviii. the Streptomyces caatingaensis protein of GenBank Accession WP_157868472.1, or
b) a translocation domain that is at least 80% identical to the translocation domain defined in a); and
C is a targeting moiety.

2-5. (canceled)

6. The recombinant polypeptide of claim 1, wherein
the translocation polypeptide comprises:
a) the translocation domain from the Austwickia chelonae protein, wherein the translocation domain comprises the amino acid sequence of SEQ ID NO: 3, or
b) a translocation domain that is at least 80% identical to the translocation domain defined in a).

7-19. (canceled)

20. The recombinant polypeptide of claim 1,
wherein the translocation polypeptide comprises:
a) the translocation domain from the Austwickia chelonae LK16-18 protein of GenBank Accession WP_162873017.1, wherein the translocation domain comprises the amino acid sequence of SEQ ID NO: 17, or
b) a translocation domain that is at least 80% identical to the translocation domain defined in a).

21-23. (canceled)

24. The recombinant polypeptide of claim 1, wherein the translocation domain is as defined in b) and is at least at least 95% identical to the translocation domain defined in a) across the full length thereof.

25. (canceled)

26. (canceled)

27. The recombinant polypeptide of claim 1, wherein the translocation polypeptide is as defined in a).

28. The recombinant polypeptide of claim 1, wherein A and B are separated by an amino acid linker.

29-32. (canceled)

33. The recombinant polypeptide of claim 1, wherein B and C are separated by an amino acid linker.

34-52. (canceled)

53. The recombinant polypeptide of claim 1, wherein the targeting moiety comprises a targeting polypeptide or aptamer.

54-58. (canceled)

59. The recombinant polypeptide of claim 53, wherein the targeting moiety comprises at least two targeting polypeptides, at least two aptamers, or a combination of a targeting polypeptide and an aptamer.

60-66. (canceled)

67. The recombinant polypeptide of claim 1, wherein the targeting moiety binds to a cell surface protein.

68. The recombinant polypeptide of claim 67, wherein the cell surface protein is lineage-specific or tissue-specific.

69. (canceled)

70. The recombinant polypeptide of claim 67, wherein the cell surface protein is expressed in a disease cell.

71. (canceled)

72. (canceled)

73. The recombinant polypeptide of claim 70, wherein the disease cell is a cancer cell.

74. The recombinant polypeptide of claim 1, wherein the cargo molecule comprises a therapeutic polypeptide.

75. The recombinant polypeptide of claim 74, wherein the therapeutic polypeptide comprises a cytotoxic polypeptide, preferably a polypeptide toxin or a functional fragment thereof.

76. (canceled)

77. The recombinant polypeptide of claim 75, wherein the cytotoxic polypeptide comprises a catalytic domain from a Chelona Toxin, wherein the catalytic domain comprises:
the amino acid sequence according to amino acid positions 1 to 186 of SEQ ID NO: 2,
the amino acid sequence according to amino acid positions 1 to 186 of SEQ ID NO: 21, or
the amino acid sequence according to amino acid positions 1 to 191 of SEQ ID NO: 35.

78-83. (canceled)

84. A nucleic acid encoding the recombinant polypeptide as defined in claim 1.

85. (canceled)

86. A pharmaceutical composition comprising the recombinant polypeptide as defined in claim 1, together with a pharmaceutically acceptable excipient, diluent, or carrier.

87. A method of delivering a cargo molecule to a cell comprising contacting the cell with the recombinant polypeptide as defined in claim 1.

88. (canceled)

89. (canceled)

90. A method treating cancer in a subject comprising administering to the subject the recombinant polypeptide as defined in claim 1.

91. (canceled)

92. (canceled)


SEQUENCE LISTING (48 sequences)

SEQ ID NO: 1 - protein, 535 aa, Corynebacterium diphtheriae
GADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 2 - protein, 539 aa, Austwickia chelonae
YANDVVLKDQSTTVDSFTSYHGAKPESFNAVLTGIKKPEKGSQGNNDPDWKGFYTTDNKHAAAGYTVSDESVLSGKAGGVVRVTYPGKTRILAVKSLSAAELKGKLGLDSAKPLIDQLNDKSFLEKYGDGANRVVLKMPFADGTEDSEFIHNWKDAEQLSVETEVRFDNLGKRGQDAMNSYMNMANCPSTSAVRAKRSPGKICLSKINWKNVREKADALTKKVHADKEFMDKLSTHHQRGEAPSVEKTTALHNALLEHESFSALKGARASGKVGAAASTAAWGVAVAQAFTDPKADALTKTAATLSVVPGLGQALGIADGIKHENTEEIVVQSISLAGLLAAQAIPVVGEAVDFGLLVYQLVETIVDLATHLSSAAANPPTEATDSVRPAVSLGLRAGWKTEEDAKLHIGSPYGMKFQRIVLSAEEGKEIPFVRAAVAVDSKFLKINGPRSFVVQNGIKTPMACFETEGNLAFCRPSRPIFLSSSSPATLHLSYVTNEHENGTIKNPTVDILGQRIVENKVITANKVSLVYKVDSSNTL

SEQ ID NO: 3 - protein, 175 aa, Austwickia chelonae
LSKINWKNVREKADALTKKVHADKEFMDKLSTHHQRGEAPSVEKTTALHNALLEHESFSALKGARASGKVGAAASTAAWGVAVAQAFTDPKADALTKTAATLSVVPGLGQALGIADGIKHENTEEIVVQSISLAGLLAAQAIPVVGEAVDFGLLVYQLVETIVDLATHLSSAAAN

SEQ ID NO: 4 - protein, 164 aa, Streptosporangium nondiastaticum
LPVDWDKVEEKAKATAKKVAQDAEHVERLPKRSPKGPTWGEAHSTAELTHAKVREVSGAHVAASAAGVGTWVYGMAKTFSDKNATTLDKVAVTGAVVPGLGQALGIADGIQHGDPEAIAVNAVALAALAAAQVVPVVGEVVDAVLLTEQLVEVLVDVFRTATAD

SEQ ID NO: 5 - protein, 165 aa, Streptomyces sp.
IPAASWEAIEHRSKEMASAVARDTEYTKSLPNRHPKGPTWSEAHTTSTSTHARVSAKSGAHVAVGAFAVGSWIYGMSETFANKNVTTLDKAAATVAIVPGIGHALGIAAALEHHDIEGVVVNAISIAALAAAQVVPVVGEIVDAALLAEQLVEVLVHVFRASTTE

SEQ ID NO: 6 - protein, 171 aa, Streptomyces sp.
LSEMDGEKVTKGAQSVADTLKGHGNLQPLVKRTRAEPRTQQYLPPTRQQIDDAISAAQRVHPGVDTAMKHLGSAQALAGFALDVSQGEPLRAAADVTAMLPRIGPGLGNALGLADSIVHDDPEGIAVNAIGLGAFIVAQAIPVIGELADLGFAAYALIETLKSMFAGPTLG

SEQ ID NO: 7 - protein, 164 aa, Streptomyces sp.
LPVDWDKVEEKAKATAKKVAQDAEHVERLPKRNPKGPTWGEAHSTAELTHAKVRAVSGAHVAATAAGVGTWVYGMAKTFSDKDATTLDKVAVTGAVVPGLGQALGIADGIQHGDPEAVAVNAVALAALAAAQVVPVVGEVVDAALLTEQLVEVLVDVFRTATAD

SEQ ID NO: 8 - protein, 164 aa, Streptomyces roseoverticillatus; identical to SEQ ID NO: 7

SEQ ID NO: 9 - protein, 159 aa, Streptomyces piniterrae
ELAKDKARDILSEAGNEVSLPQRDSDGLSKQEITATAEATRSKLGTGVHGAVSAAMVADWAHDVARTFADPKATKLDKAAAVTAIAPVIGQAVNIADGIQHHDKKTIVVNSLVLAAVVAAQAVPAVGEVVDAAIVADFVVEKLVGWFTPTAKPGPEHVA

SEQ ID NO: 10 - protein, 164 aa, Streptomyces sp.
LPVDWDKVEEKAKATAKKVAQDAEHAERLPKRNPKGPTWGEAHSTAELTHAKVREVSGTHVAATAAGVGTWVYGMAKTFSDKDATTLDKVAVTGAVVPGLGQALGIADGIQHGDPEAVAVNAVALAALAAAQVVPVVGEVVDAVLLTEQLVEVLVDVFRAATAD

SEQ ID NO: 11 - protein, 164 aa, Streptomyces klenkii
LPVDWDKVEEKAKATAKKVAQDAEHVEKLPKRSPKGPTWGEAHSTAELSHAKVREVSGAHVAASAAGVGTWVYGMAKTFSDKDATTLDKVAVTGAVVPGLGQALGIADGIQHGDPEAIAVNAVALAALAAAQVVPVVGEVVDAVLLTEQLVEVLVDVFRTATAD

SEQ ID NO: 12 - protein, 168 aa, Streptomyces albireticuli
GINVAAYKADIEKLIKDVYEDPNFSDLKNRTGGPQKDKDTLKGYYERLKPKVETLRPLKAGVSSAVGAAGAISWAIGVADAFTSENVSSFDKAAAVTAIVPGLGECVGIANAIDKRDPEGLIINTISMAALMASAAVPVLAPIGVALDAGLAAAQGVATVLEYLEIGQ

SEQ ID NO: 13 - protein, 176 aa, Streptacidiphilus pinicola
KVNWKDAYDRSMNIAQDIDGSAEFRATAPARPAAGKAMPEADVTRLVSTSEDFLEKSTKTSGVKKALKAINNQKMISWGSLLSNALANSHTWSDKNATNLDKAYAVVGGVPVLGEVIGIASGIDKQDAESIAVNTLSLVGIVAATVCPPLGATVEFVMIGYTAIKLMLSWFTVETI

SEQ ID NO: 14 - protein, 165 aa, Seinonella peptonophila
LDWEKIKTESQRIVKQIIEEHPELQSHSKNAVTDKEKLQKIYNDYAPKIDKLSSLKEGVSRATTALNIASWAAGLAETFSNKNADGLDKAAAVTAIIPGLGQAVGIANGIEKHDGEAIAINSIALSALVVAQAIPIVGEIADVVGAGLILAGGLAQLIQSVSPDT

SEQ ID NO: 15 - protein, 178 aa, Longimycelium tulufanense
ERKTLDKLDRKKIETRAKQVISKLAQDGDLRRELPRRTTTGHAHEEVRGVLAKSRGALHEIHGAATTVAMPLATVAWVEDMARVFREKNATTLDKAATVSEIVPVAGQVLGMADGIAHRDAETVAVNAVVLAAIAVSQAVPVVGELVDLGLTAYAVVDVVVRLFGPAREVPITQESYW

SEQ ID NO: 16 - protein, 175 aa, Austwickia sp.
LSNIKWERVRQKSEELIKKLKDDENVKKAVQERKEGVKPTSDDLQNLHKALIDHESFKELKSVHSNGVKAMDAANAALWGANVARVFSDSKSDGLEKATAALAAVPGLGQVMGVADGVTHHNTEEVVVQSVALAGFIAAQAIPVVGEIVDIGVLAYQFVEGVIDLSRQMMTSNAR

SEQ ID NO: 17 - protein, 176 aa, Austwickia chelonae
LSKVKWEQVREKSKKIIDNVKDNPEFMKKLSAHHERGSAPTTEKITALHNELLDHESFSALKGARSSAGTAATAASAAAWGLAVAQAFTNPKADDLTKATAVLSAVPGLGQALGIADGIKHHNTEEIVVQSISLTALIAAQAIPVVGELVDFGLLAYQLVESIIDLTRQLSVITAN

SEQ ID NO: 18 - protein, 510 aa, Vibrio vulnificus
GDKTKVVVDLAQIFTVQELKERAKVFAKPIGASYQGILDQLDLVHQAKGRDQIAASFELNKKINDYIAEHPTSGRNQALTQLKEQVTSALFIGKMQVAQAGIDAIAQTRPELAARIFMVAIEEANGKHVGLTDMMVRWANEDPYLAPKHGYKGETPSDLGFDAKYHVDLGEHYADFKQWLETSQSNGLLSKATLDESTKTVHLGYSYQELQDLTGAESVQMAFYFLKEAAKKADPISGDSAEMILLKKFADQSYLSQLDSDRMDQIEGIYRSSHETDIDAWDRRYSGTGYDELTNKLASATGVDEQLAVLLDDRKGLLIGEVHGSDVNGLRFVNEQMDALKKQGVTVIGLEHLRSDLAQPLIDRYLATGVMSSELSAMLKTKHLDVTLFENARANGMRIVALDANSSARPNVQGTEHGLMYRAGAANNIAVEVLQNLPDGEKFVAIYGKAHLQSHKGIEGFVPGITHRLDLPALKVSDSNQFTVEQDDVSLRVVYDDVANKPKITFKGSL

SEQ ID NO: 19 - protein, 58 aa, synthetic construct
AEAKYAKEKYNAYYEIWQLPNLTKYQKAAFIGKLQDDPSQSSELLSEAKKLNDSQAPK

SEQ ID NO: 20 - protein, 20 aa, synthetic construct
NAVPNLRGDLQVLAQKVART

SEQ ID NO: 21 - protein, 540 aa, Austwickia chelonae
YANDAVIADQSKTVDSFTSYHGAKPESFESVLAGIKKPESGSQGNHDPEWKGFYTTDNKHAAAGYTVSDESVMTGKAGGVVKVTYPGKTRVLAVKPLSATELKTYLGLAADKPLIDQLNNKDFINKFGEGASRVVLQMPFADGTSDVEYIHNWEDATQLQVATEVRFDNLGKRGQDEMNRYMNLANCPSVSAVRVKRNPAKLCLSKVKWEQVREKSKKIIDNVKDNPEFMKKLSAHHERGSAPTTEKITALHNELLDHESFSALKGARSSAGTAATAASAAAWGLAVAQAFTNPKADDLTKATAVLSAVPGLGQALGIADGIKHHNTEEIVVQSISLTALIAAQAIPVVGELVDFGLLAYQLVESIIDLTRQLSVITANPPTEVTHSSKLAKSNGLLAGWKTDQDGVLSLGAPHGMKTQLISMSAEKGQEIPFTGAMIAVKKDLLTVNTPRVFVVQNGIRIPLRCSAAGETLTFCRPNHPVWLSEQHQATLHLSYVTQENESEKIDNPTVDILGQKIVDNKVSTANKVSLVYEVDRSNKL

SEQ ID NO: 22 - protein, 840 aa, synthetic construct
GDKTKVVVDLAQIFTVQELKERAKVFAKPIGASYQGILDQLDLVHQAKGRDQIAASFELNKKINDYIAEHPTSGRNQALTQLKEQVTSALFIGKMQVAQAGIDAIAQTRPELAARIFMVAIEEANGKHVGLTDMMVRWANEDPYLAPKHGYKGETPSDLGFDAKYHVDLGEHYADFKQWLETSQSNGLLSKATLDESTKTVHLGYSYQELQDLTGAESVQMAFYFLKEAAKKADPISGDSAEMILLKKFADQSYLSQLDSDRMDQIEGIYRSSHETDIDAWDRRYSGTGYDELTNKLASATGVDEQLAVLLDDRKGLLIGEVHGSDVNGLRFVNEQMDALKKQGVTVIGLEHLRSDLAQPLIDRYLATGVMSSELSAMLKTKHLDVTLFENARANGMRIVALDANSSARPNVQGTEHGLMYRAGAANNIAVEVLQNLPDGEKFVAIYGKAHLQSHKGIEGFVPGITHRLDLPALKVSDSNQFTVEQDDVSLRVVYDDVANKPKITFKGSLGGGGSGGGGSCAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFGGGGSGGGGSAEAKYAKEKYNAYYEIWQLPNLTKYQKAAFIGKLQDDPSQSSELLSEAKKLNDSQAPKGGGGSGGGGSNAVPNLRGDLQVLAQKVARTRQALVPRGSAWSHPQFEK

SEQ ID NO: 23 - protein, 839 aa, synthetic construct
GDKTKVVVDLAQIFTVQELKERAKVFAKPIGASYQGILDQLDLVHQAKGRDQIAASFELNKKINDYIAEHPTSGRNQALTQLKEQVTSALFIGKMQVAQAGIDAIAQTRPELAARIFMVAIEEANGKHVGLTDMMVRWANEDPYLAPKHGYKGETPSDLGFDAKYHVDLGEHYADFKQWLETSQSNGLLSKATLDESTKTVHLGYSYQELQDLTGAESVQMAFYFLKEAAKKADPISGDSAEMILLKKFADQSYLSQLDSDRMDQIEGIYRSSHETDIDAWDRRYSGTGYDELTNKLASATGVDEQLAVLLDDRKGLLIGEVHGSDVNGLRFVNEQMDALKKQGVTVIGLEHLRSDLAQPLIDRYLATGVMSSELSAMLKTKHLDVTLFENARANGMRIVALDANSSARPNVQGTEHGLMYRAGAANNIAVEVLQNLPDGEKFVAIYGKAHLQSHKGIEGFVPGITHRLDLPALKVSDSNQFTVEQDDVSLRVVYDDVANKPKITFKGSLGGGGSGGGGSCAGNRVRRSVGSSLSCLSKINWKNVREKADALTKKVHADKEFMDKLSTHHQRGEAPSVEKTTALHNALLEHESFSALKGARASGKVGAAASTAAWGVAVAQAFTDPKADALTKTAATLSVVPGLGQALGIADGIKHENTEEIVVQSISLAGLLAAQAIPVVGEAVDFGLLVYQLVETIVDLATHLSSAAANPAYSPGHKTQPFGGGGSGGGGSAEAKYAKEKYNAYYEIWQLPNLTKYQKAAFIGKLQDDPSQSSELLSEAKKLNDSQAPKGGGGSGGGGSNAVPNLRGDLQVLAQKVARTRQALVPRGSAWSHPQFEK

SEQ ID NO: 24 - protein, 840 aa, synthetic construct
GDKTKVVVDLAQIFTVQELKERAKVFAKPIGASYQGILDQLDLVHQAKGRDQIAASFELNKKINDYIAEHPTSGRNQALTQLKEQVTSALFIGKMQVAQAGIDAIAQTRPELAARIFMVAIEEANGKHVGLTDMMVRWANEDPYLAPKHGYKGETPSDLGFDAKYHVDLGEHYADFKQWLETSQSNGLLSKATLDESTKTVHLGYSYQELQDLTGAESVQMAFYFLKEAAKKADPISGDSAEMILLKKFADQSYLSQLDSDRMDQIEGIYRSSHETDIDAWDRRYSGTGYDELTNKLASATGVDEQLAVLLDDRKGLLIGEVHGSDVNGLRFVNEQMDALKKQGVTVIGLEHLRSDLAQPLIDRYLATGVMSSELSAMLKTKHLDVTLFENARANGMRIVALDANSSARPNVQGTEHGLMYRAGAANNIAVEVLQNLPDGEKFVAIYGKAHLQSHKGIEGFVPGITHRLDLPALKVSDSNQFTVEQDDVSLRVVYDDVANKPKITFKGSLGGGGSGGGGSCAGNRVRRSVGSSLSCLSKVKWEQVREKSKKIIDNVKDNPEFMKKLSAHHERGSAPTTEKITALHNELLDHESFSALKGARSSAGTAATAASAAAWGLAVAQAFTNPKADDLTKATAVLSAVPGLGQALGIADGIKHHNTEEIVVQSISLTALIAAQAIPVVGELVDFGLLAYQLVESIIDLTRQLSVITANPAYSPGHKTQPFGGGGSGGGGSAEAKYAKEKYNAYYEIWQLPNLTKYQKAAFIGKLQDDPSQSSELLSEAKKLNDSQAPKGGGGSGGGGSNAVPNLRGDLQVLAQKVARTRQALVPRGSAWSHPQFEK

SEQ ID NO: 25 - protein, 839 aa, synthetic construct
GDKTKVVVDLAQIFTVQELKERAKVFAKPIGASYQGILDQLDLVHQAKGRDQIAASFELNKKINDYIAEHPTSGRNQALTQLKEQVTSALFIGKMQVAQAGIDAIAQTRPELAARIFMVAIEEANGKHVGLTDMMVRWANEDPYLAPKHGYKGETPSDLGFDAKYHVDLGEHYADFKQWLETSQSNGLLSKATLDESTKTVHLGYSYQELQDLTGAESVQMAFYFLKEAAKKADPISGDSAEMILLKKFADQSYLSQLDSDRMDQIEGIYRSSHETDIDAWDRRYSGTGYDELTNKLASATGVDEQLAVLLDDRKGLLIGEVHGSDVNGLRFVNEQMDALKKQGVTVIGLEHLRSDLAQPLIDRYLATGVMSSELSAMLKTKHLDVTLFENARANGMRIVALDANSSARPNVQGTEHGLMYRAGAANNIAVEVLQNLPDGEKFVAIYGKAHLQSHKGIEGFVPGITHRLDLPALKVSDSNQFTVEQDDVSLRVVYDDVANKPKITFKGSLGGGGSGGGGSCAGNRVRRSVGSSLSCLSNIKWERVRQKSEELIKKLKDDENVKKAVQERKEGVKPTSDDLQNLHKALIDHESFKELKSVHSNGVKAMDAANAALWGANVARVFSDSKSDGLEKATAALAAVPGLGQVMGVADGVTHHNTEEVVVQSVALAGFIAAQAIPVVGEIVDIGVLAYQFVEGVIDLSRQMMTSNARPAYSPGHKTQPFGGGGSGGGGSAEAKYAKEKYNAYYEIWQLPNLTKYQKAAFIGKLQDDPSQSSELLSEAKKLNDSQAPKGGGGSGGGGSNAVPNLRGDLQVLAQKVARTRQALVPRGSAWSHPQFEK

SEQ ID NO: 26 - protein, 828 aa, synthetic construct
GDKTKVVVDLAQIFTVQELKERAKVFAKPIGASYQGILDQLDLVHQAKGRDQIAASFELNKKINDYIAEHPTSGRNQALTQLKEQVTSALFIGKMQVAQAGIDAIAQTRPELAARIFMVAIEEANGKHVGLTDMMVRWANEDPYLAPKHGYKGETPSDLGFDAKYHVDLGEHYADFKQWLETSQSNGLLSKATLDESTKTVHLGYSYQELQDLTGAESVQMAFYFLKEAAKKADPISGDSAEMILLKKFADQSYLSQLDSDRMDQIEGIYRSSHETDIDAWDRRYSGTGYDELTNKLASATGVDEQLAVLLDDRKGLLIGEVHGSDVNGLRFVNEQMDALKKQGVTVIGLEHLRSDLAQPLIDRYLATGVMSSELSAMLKTKHLDVTLFENARANGMRIVALDANSSARPNVQGTEHGLMYRAGAANNIAVEVLQNLPDGEKFVAIYGKAHLQSHKGIEGFVPGITHRLDLPALKVSDSNQFTVEQDDVSLRVVYDDVANKPKITFKGSLGGGGSGGGGSCAGNRVRRSVGSSLSCLPVDWDKVEEKAKATAKKVAQDAEHVEKLPKRSPKGPTWGEAHSTAELSHAKVREVSGAHVAASAAGVGTWVYGMAKTFSDKDATTLDKVAVTGAVVPGLGQALGIADGIQHGDPEAIAVNAVALAALAAAQVVPVVGEVVDAVLLTEQLVEVLVDVFRTATADPAYSPGHKTQPFGGGGSGGGGSAEAKYAKEKYNAYYEIWQLPNLTKYQKAAFIGKLQDDPSQSSELLSEAKKLNDSQAPKGGGGSGGGGSNAVPNLRGDLQVLAQKVARTRQALVPRGSAWSHPQFEK

SEQ ID NO: 27 - protein, 829 aa, synthetic construct
GDKTKVVVDLAQIFTVQELKERAKVFAKPIGASYQGILDQLDLVHQAKGRDQIAASFELNKKINDYIAEHPTSGRNQALTQLKEQVTSALFIGKMQVAQAGIDAIAQTRPELAARIFMVAIEEANGKHVGLTDMMVRWANEDPYLAPKHGYKGETPSDLGFDAKYHVDLGEHYADFKQWLETSQSNGLLSKATLDESTKTVHLGYSYQELQDLTGAESVQMAFYFLKEAAKKADPISGDSAEMILLKKFADQSYLSQLDSDRMDQIEGIYRSSHETDIDAWDRRYSGTGYDELTNKLASATGVDEQLAVLLDDRKGLLIGEVHGSDVNGLRFVNEQMDALKKQGVTVIGLEHLRSDLAQPLIDRYLATGVMSSELSAMLKTKHLDVTLFENARANGMRIVALDANSSARPNVQGTEHGLMYRAGAANNIAVEVLQNLPDGEKFVAIYGKAHLQSHKGIEGFVPGITHRLDLPALKVSDSNQFTVEQDDVSLRVVYDDVANKPKITFKGSLGGGGSGGGGSCAGNRVRRSVGSSLSCIPAASWEAIEHRSKEMASAVARDTEYTKSLPNRHPKGPTWSEAHTTSTSTHARVSAKSGAHVAVGAFAVGSWIYGMSETFANKNVTTLDKAAATVAIVPGIGHALGIAAALEHHDIEGVVVNAISIAALAAAQVVPVVGEIVDAALLAEQLVEVLVHVFRASTTEPAYSPGHKTQPFGGGGSGGGGSAEAKYAKEKYNAYYEIWQLPNLTKYQKAAFIGKLQDDPSQSSELLSEAKKLNDSQAPKGGGGSGGGGSNAVPNLRGDLQVLAQKVARTRQALVPRGSAWSHPQFEK

SEQ ID NO: 28 - protein, 842 aa, synthetic construct
GDKTKVVVDLAQIFTVQELKERAKVFAKPIGASYQGILDQLDLVHQAKGRDQIAASFELNKKINDYIAEHPTSGRNQALTQLKEQVTSALFIGKMQVAQAGIDAIAQTRPELAARIFMVAIEEANGKHVGLTDMMVRWANEDPYLAPKHGYKGETPSDLGFDAKYHVDLGEHYADFKQWLETSQSNGLLSKATLDESTKTVHLGYSYQELQDLTGAESVQMAFYFLKEAAKKADPISGDSAEMILLKKFADQSYLSQLDSDRMDQIEGIYRSSHETDIDAWDRRYSGTGYDELTNKLASATGVDEQLAVLLDDRKGLLIGEVHGSDVNGLRFVNEQMDALKKQGVTVIGLEHLRSDLAQPLIDRYLATGVMSSELSAMLKTKHLDVTLFENARANGMRIVALDANSSARPNVQGTEHGLMYRAGAANNIAVEVLQNLPDGEKFVAIYGKAHLQSHKGIEGFVPGITHRLDLPALKVSDSNQFTVEQDDVSLRVVYDDVANKPKITFKGSLGGGGSGGGGSCAGNRVRRSVGSSLSCERKTLDKLDRKKIETRAKQVISKLAQDGDLRRELPRRTTTGHAHEEVRGVLAKSRGALHEIHGAATTVAMPLATVAWVEDMARVFREKNATTLDKAATVSEIVPVAGQVLGMADGIAHRDAETVAVNAVVLAAIAVSQAVPVVGELVDLGLTAYAVVDVVVRLFGPAREVPITQESYWPAYSPGHKTQPFGGGGSGGGGSAEAKYAKEKYNAYYEIWQLPNLTKYQKAAFIGKLQDDPSQSSELLSEAKKLNDSQAPKGGGGSGGGGSNAVPNLRGDLQVLAQKVARTRQALVPRGSAWSHPQFEK

SEQ ID NO: 29 - protein, 823 aa, synthetic construct
GDKTKVVVDLAQIFTVQELKERAKVFAKPIGASYQGILDQLDLVHQAKGRDQIAASFELNKKINDYIAEHPTSGRNQALTQLKEQVTSALFIGKMQVAQAGIDAIAQTRPELAARIFMVAIEEANGKHVGLTDMMVRWANEDPYLAPKHGYKGETPSDLGFDAKYHVDLGEHYADFKQWLETSQSNGLLSKATLDESTKTVHLGYSYQELQDLTGAESVQMAFYFLKEAAKKADPISGDSAEMILLKKFADQSYLSQLDSDRMDQIEGIYRSSHETDIDAWDRRYSGTGYDELTNKLASATGVDEQLAVLLDDRKGLLIGEVHGSDVNGLRFVNEQMDALKKQGVTVIGLEHLRSDLAQPLIDRYLATGVMSSELSAMLKTKHLDVTLFENARANGMRIVALDANSSARPNVQGTEHGLMYRAGAANNIAVEVLQNLPDGEKFVAIYGKAHLQSHKGIEGFVPGITHRLDLPALKVSDSNQFTVEQDDVSLRVVYDDVANKPKITFKGSLGGGGSGGGGSCAGNRVRRSVGSSLSCELAKDKARDILSEAGNEVSLPQRDSDGLSKQEITATAEATRSKLGTGVHGAVSAAMVADWAHDVARTFADPKATKLDKAAAVTAIAPVIGQAVNIADGIQHHDKKTIVVNSLVLAAVVAAQAVPAVGEVVDAAIVADFVVEKLVGWFTPTAKPGPEHVAPAYSPGHKTQPFGGGGSGGGGSAEAKYAKEKYNAYYEIWQLPNLTKYQKAAFIGKLQDDPSQSSELLSEAKKLNDSQAPKGGGGSGGGGSNAVPNLRGDLQVLAQKVARTRQALVPRGSAWSHPQFEK

SEQ ID NO: 30 - protein, 840 aa, synthetic construct
GDKTKVVVDLAQIFTVQELKERAKVFAKPIGASYQGILDQLDLVHQAKGRDQIAASFELNKKINDYIAEHPTSGRNQALTQLKEQVTSALFIGKMQVAQAGIDAIAQTRPELAARIFMVAIEEANGKHVGLTDMMVRWANEDPYLAPKHGYKGETPSDLGFDAKYHVDLGEHYADFKQWLETSQSNGLLSKATLDESTKTVHLGYSYQELQDLTGAESVQMAFYFLKEAAKKADPISGDSAEMILLKKFADQSYLSQLDSDRMDQIEGIYRSSHETDIDAWDRRYSGTGYDELTNKLASATGVDEQLAVLLDDRKGLLIGEVHGSDVNGLRFVNEQMDALKKQGVTVIGLEHLRSDLAQPLIDRYLATGVMSSELSAMLKTKHLDVTLFENARANGMRIVALDANSSARPNVQGTEHGLMYRAGAANNIAVEVLQNLPDGEKFVAIYGKAHLQSHKGIEGFVPGITHRLDLPALKVSDSNQFTVEQDDVSLRVVYDDVANKPKITFKGSLGGGGSGGGGSCAGNRVRRSVGSSLSCKVNWKDAYDRSMNIAQDIDGSAEFRATAPARPAAGKAMPEADVTRLVSTSEDFLEKSTKTSGVKKALKAINNQKMISWGSLLSNALANSHTWSDKNATNLDKAYAVVGGVPVLGEVIGIASGIDKQDAESIAVNTLSLVGIVAATVCPPLGATVEFVMIGYTAIKLMLSWFTVETIPAYSPGHKTQPFGGGGSGGGGSAEAKYAKEKYNAYYEIWQLPNLTKYQKAAFIGKLQDDPSQSSELLSEAKKLNDSQAPKGGGGSGGGGSNAVPNLRGDLQVLAQKVARTRQALVPRGSAWSHPQFEK

SEQ ID NO: 31 - protein, 505 aa, synthetic construct
GADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFGGGGSGGGGSAEAKYAKEKYNAYYEIWQLPNLTKYQKAAFIGKLQDDPSQSSELLSEAKKLNDSQAPKGGGGSGGGGSNAVPNLRGDLQVLAQKVARTRQALVPRGSAWSHPQFEK

SEQ ID NO: 32 - protein, 507 aa, synthetic construct
YANDAVIADQSKTVDSFTSYHGAKPESFESVLAGIKKPESGSQGNHDPEWKGFYTTDNKHAAAGYTVSDESVMTGKAGGVVKVTYPGKTRVLAVKPLSATELKTYLGLAADKPLIDQLNNKDFINKFGEGASRVVLQMPFADGTSDVEYIHNWEDATQLQVATEVRFDNLGKRGQDEMNRYMNLANCPSVSAVRVKRNPAKLCLSKVKWEQVREKSKKIIDNVKDNPEFMKKLSAHHERGSAPTTEKITALHNELLDHESFSALKGARSSAGTAATAASAAAWGLAVAQAFTNPKADDLTKATAVLSAVPGLGQALGIADGIKHHNTEEIVVQSISLTALIAAQAIPVVGELVDFGLLAYQLVESIIDLTRQLSVITANPPTEVTHSSKLAGGGGSGGGGSAEAKYAKEKYNAYYEIWQLPNLTKYQKAAFIGKLQDDPSQSSELLSEAKKLNDSQAPKGGGGSGGGGSNAVPNLRGDLQVLAQKVARTRQALVPRGSAWSHPQFEK

SEQ ID NO: 33 - protein, 16 aa, Corynebacterium diphtheriae
CAGNRVRRSVGSSLSC

SEQ ID NO: 34 - protein, 12 aa, Corynebacterium diphtheriae
PAYSPGHKTQPF

SEQ ID NO: 35 - protein, 637 aa, Austwickia sp.
GAFASPISPAIDKEKSKVVENFTSYHGTKHQYIENVLKGINKPETGSQGNHDPDWKGFYTTDSKYAAAGYTPDEENPMSGKAGGVVKVSYTGKTKILALKDMPAGKIKGHLGLDPNKPLTEQIKDQEFINKYGEGADRVVLQMPFAEGSSEVEYINNWDNAKDLKVEPEVRFDQHGKRGQDAMYEYMALASCESAGKAKRSVSKTCLSNIKWERVRQKSEELIKKLKDDENVKKAVQERKEGVKPTSDDLQNLHKALIDHESFKELKSVHSNGVKAMDAANAALWGANVARVFSDSKSDGLEKATAALAAVPGLGQVMGVADGVTHHNTEEVVVQSVALAGFIAAQAIPVVGEIVDIGVLAYQFVEGVIDLSRQMMTSNARGIAEKRQVVDIKDANGLSGGWLTPQDSKIHLPPHYGTKTQRLAVSANGDHNLPITGFKTSINPKFFNINESGSFIVQNGLKVPMNCSEEASKTEAVCIPVHPIWLTDKNNAIIHVSYQTKSPEGSRIDNPRLEVWGQIMHTEEGPKTNTINLGYSVDQEMPCYSSIKFENGGDYVGRLAVNSKDGKVTHSKMSGMRDKDKVDLSAFNSGEVLTTEISPTGGGAPTPGPSIKYCSDGGIEASIKSWGTKDKAFLGFN

SEQ ID NO: 36 - protein, 166 aa, Klebsiella aerogenes
GLNWGSIREKAVDKAKSVTSDKKFINSLPERGKNSPANKNDINTIYESSANHSTLKGGSEVIHKASLLLWVANTADVLTSNSSSTLDKATAIIGIVPGIGDAFSLADSIEHDDVEGIITSSIALAAFTVAQAVPVVGELVDLALLTEAAISSLVNVISNYIQSTQR

SEQ ID NO: 37 - protein, 164 aa, Streptomyces sp.
ADLDWEKVRSRAKETARQVAQDSEHVSGLPERRAEGLTKSEAHAVAERTHAKVSEVSGTRVAATAFGVGTWAYGMAQTFTDQNATTLDKVAVTTAIVPGVGNALGIADGIEHHDPEAVAVNAVALAALVAAQAVPVVGELVDTVLLTEQIVETFVNIFQRATAA

SEQ ID NO: 38 - protein, 162 aa, Crossiella cryophila
ETADSLGELFHQGMTIAGEAVAELDLEEALKSQKVAKGDARAVVAGKVAAVGKQVGKSSALAKFKEVLKPSKGVIALWAVSALYTLAQEDVSTVDKAEAVLAIVPILGPLFSFGNSYRKEDVEGMVISTVALAATLIAFLCPPVGAGIGVGLAVYQVVRAFC

SEQ ID NO: 39 - protein, 161 aa, Allokutzneria sp.
SLVKGKTDAQAVSAVKQLFENSKTVATSLVADSDMADAIKDAKKAAKVGVAPPVDKVKKVTDPLVEKALKSKSFANTKAGKYLKNAQPSKAGTALWAATTVATFLDGSSSASDKAVAAVGIIPVVGQLASLIHTTIKDPSDVEGIVTGTIGLAAAVITIAC

SEQ ID NO: 40 - protein, 163 aa, Allokutzneria albata
KAIGATRESARKFVANILSKSEKATTTLAASNDYKSSMKGFSESFAKLRKAAAETKKPIPTADIKKLLTPHLDKVSTVKDFKKYFTNLKPSQGSAATWLVNLAATVANEESSKADVAVAAAGIIPVVGQVAAIANSVSQGDVEGTVTSAIALAANIAIAVGAG

SEQ ID NO: 41 - protein, 222 aa, Streptomyces sp.
CDIGVRDHAEVARKHQERLRAHGDDVEYVTTLDEFEKNPKSLLSPLRETNGVLNDAAAAELGAKDFEAFSTRVTEALGRDLRAVHGLSTDTTLLAKVARQAGRAAELGAKALPYVGIAATGYAIAEDVKAGDYANLAFDSVAEGLQVAMVAQPELVPLLEPVLFAEQLAQIVYDEIAGWFKRQEQLDHDRKQWDSAAEDLVEHRDGQWRDHLVGQALQKLFP

SEQ ID NO: 42 - protein, 219 aa, Streptomyces sp.
CGWGDDGVAHTQRERLRAQGGDVEYVTSLEEFEKNPKALLSPLRETNKVLNDAASAELGAKDFETFSARVGAAFSRDLRAVQGLTTDTGLLAGVGRQVGRAGDLGVKVLPYVGIAVTGYAVAEDAKAGDYANLAFDSVAEGLQVAMVAQPEVAPLLEPALLAEQLAQLVYNEVAGWIAEQKQLEHDRAQWDGAVKDLVAHRDTQWRDRLVGQAVQELFP

SEQ ID NO: 43 - protein, 222 aa, Streptomyces syringium
CGIAAADDAKVARTHQERLRAQGADVEYVTSLEEFEKNPKALMSPLRETSKVLNGAAAAELGAKDFEAFSTQVTEALGRDLKAVHGLTTDTGLLAEVGRRVGRAGELGVKILPYAGIAATGYAVAEDAKAGDYGNLAFDSVAEGLQVAMVAQPELTPLLEPALLAEQLAQLVYDEVAGWIAEQKQLAHDRGQWDDAVKDLVAHRDTQWRNRLEGQALQELFP

SEQ ID NO: 44 - protein, 179 aa, unidentified
  source 1..179: note = Pseudonocardiaceae bacterium
RIGKAALAKATAEAQADAQKILKDPSFKPPHPAPHQKLTDAELTQVANDLAKRVSALHPGARDLTGLGKGLAKALKGVISGVTAGIQAAEIAESAAGSNSGDLEKVCSALGPIPIVGDIAGIVNAAAEGDAVEIVKNVLTLGLSMVSVAFPPLAPVADAVAFVLEVAVFLGKYLWGLLF

SEQ ID NO: 45 - protein, 194 aa, Actinokineospora bangkokensis
SLSQDQLDQAVADAQATAHQVIDDPRSFELPELAKGQVLSEQELAALANGVAQDVAAANSGGERAIVPAYVTRALSKAIPAIAEHGHDAFLKVSAVEGVLTEGNGALAKVTSVVGPVPVLGSLAGIVNEAVGISADDPPAAIAQKALEIAKNVAGVALTVMAIAFPPFAPVAALATAAWEVGAALGKYLLGFLL

SEQ ID NO: 46 - protein, 170 aa, Streptomyces eurocidicus
CETADGGDTAEEGKQPGEGRQATESDSTEGRNPQGEEGPAEAGKPVADEEWVARVQRERLARHGSDVEYVTSVAELEKNPKAVLSPLRETNTTLNKARDKPLTAAEYEAFASKAAEAMSKDLKGVQAASTDSTVLAKVGAKLARGGSIAVKVGRPVLKRRLLRSSAASSR

SEQ ID NO: 47 - protein, 156 aa, Streptomyces pathocidini
GTGRQDGGFSSEGREGAVTKEDLKGDPQKVVELNRSRIARHGADVDYVTSLDALAANPKAVLIPLRNSSQTLDAALGKSHLTVDEFDRFSAELTQSFGRDLQAVENLSTSEKLLSSIGKGARAGGEGLLKVVPFAGVAASSAALAEDLKSGENGDA

SEQ ID NO: 48 - protein, 149 aa, Streptomyces caatingaensis
SADIARTHRERLRAHGDDVEYVTSLAEFEKNPKALLSPLRETGKVLNDAGNAGLSAGDFETFSARVGEAFERDLRAAHELTTDTGLLARVGAEAGRAAELGAKALPFVGIAATGYAVAEDVKSGDYANLAFDSVAEGLQVAMTAQPEFA